SEQUENCE LISTING (67 sequences)

SEQ ID NO: 1 - DNA, 2212 bp, Homo sapiens
ccgccgccacagctgcagtcagcaccgtcaccccagcagcatccgccgcctgcaccgcgcgtgcggcccgccccggcctgaccccgccgccgaacccggcgccagccatggagcccgaagccccccgtcgccgccacacccatcagcgcggctacctgctgacacggaaccctcacctcaacaaggacttggcctttaccctggaagagagacagcaattgaacattcatggattgttgccaccttccttcaacagtcaggagatccaggttcttagagtagtaaaaaatttcgagcatctgaactctgactttgacaggtatcttctcttaatggatctccaagatagaaatgaaaaactcttttatagagtgctgacatctgacattgagaaattcatgcctattgtttatactcccactgtgggtctggcttgccaacaatatagtttggtgtttcggaagccaagaggtctctttattactatccacgatcgagggcatattgcttcagttctcaatgcatggccagaagatgtcatcaaggccattgtggtgactgatggagagcgtattcttggcttgggagaccttggctgtaatggaatgggcatccctgtgggtaaattggctctatatacagcttgcggagggatgaatcctcaagaatgtctgcctgtcattctggatgtgggaaccgaaaatgaggagttacttaaagatccactctacattggactacggcagagaagagtaagaggttctgaatatgatgattttttggacgaattcatggaggcagtttcttccaagtatggcatgaattgccttattcagtttgaagattttgccaatgtgaatgcatttcgtctcctgaacaagtatcgaaaccagtattgcacattcaatgatgatattcaaggaacagcatctgttgcagttgcaggtctccttgcagctcttcgaataaccaagaacaaactgtctgatcaaacaatactattccaaggagctggagaggctgccctagggattgcacacctgattgtgatggccttggaaaaagaaggtttaccaaaagagaaagccatcaaaaagatatggctggttgattcaaaaggattaatagttaagggacgtgcttccttaacacaagagaaagagaagtttgcccatgaacatgaagaaatgaagaacctagaagccattgttcaagaaataaaaccaactgccctcataggagttgctgcaattggtggtgcattctcagaacaaattctcaaagatatggctgccttcaatgaacggcctattatttttgctttgagtaatccaactagcaaagcagaatgttctgcagagcagtgctacaaaataaccaagggacgtgcaatttttgccagtggcagtccttttgatccagtcactcttccaaatggacagaccctatatcctggccaaggcaacaattcctacgtgttccctggagttgctcttggtgttgtggcgtgtggattgaggcagatcacagataatattttcctcactactgctgaggttatagctcagcaagtgtcagataaacacttggaagagggtcggctttatcctcctttgaataccattagagatgtttctctgaaaattgcagaaaagattgtgaaagatgcataccaagaaaagacagccacagtttatcctgaaccgcaaaacaaagaagcatttgtccgctcccagatgtatagtactgattatgaccagattctacctgattgttattcttggcctgaagaggtgcagaaaatacagaccaaagttgaccagtaggataatagcaaacatttctaactctattaatgaggtctttaaacctttcataatttttaaaggttggaatcttttataatgattcataagacacttagattaagattttactttaacagtctaaaaattgatagaagaatatcgatataaattgggataaacatcacatgagacaattttgcttcactttgccttctggttatttatggtttctgtctgaattattctgcctacgttctctttaaaagctgttgtacgtactacggagaaactcatcatttttatacaggacactaatgggaagaccaaaattactaataaattgaaataaccaacattaaaactcataattattttgttgaccattttgttaaaatctacttttc

SEQ ID NO: 2 - protein, 572 aa, Homo sapiens
MEPEAPRRRHTHQRGYLLTRNPHLNKDLAFTLEERQQLNIHGLLPPSFNSQEIQVLRVVKNFEHLNSDFDRYLLLMDLQDRNEKLFYRVLTSDIEKFMPIVYTPTVGLACQQYSLVFRKPRGLFITIHDRGHIASVLNAWPEDVIKAIVVTDGERILGLGDLGCNGMGIPVGKLALYTACGGMNPQECLPVILDVGTENEELLKDPLYIGLRQRRVRGSEYDDFLDEFMEAVSSKYGMNCLIQFEDFANVNAFRLLNKYRNQYCTFNDDIQGTASVAVAGLLAALRITKNKLSDQTILFQGAGEAALGIAHLIVMALEKEGLPKEKAIKKIWLVDSKGLIVKGRASLTQEKEKFAHEHEEMKNLEAIVQEIKPTALIGVAAIGGAFSEQILKDMAAFNERPIIFALSNPTSKAECSAEQCYKITKGRAIFASGSPFDPVTLPNGQTLYPGQGNNSYVFPGVALGVVACGLRQITDNIFLTTAEVIAQQVSDKHLEEGRLYPPLNTIRDVSLKIAEKIVKDAYQEKTATVYPEPQNKEAFVRSQMYSTDYDQILPDCYSWPEEVQKIQTKVDQ

SEQ ID NO: 3 - DNA, 1930 bp, Homo sapiens
cggaaggagaggaccgaggtctgccaaggaccatgggtgccgcgctggggacaggcacgcggctggctccctggccgggccgggcctgcggcgccctcccgcgctggacacccaccgcgcccgcccaaggctgccactccaagcctggcccggcgcgccctgtgcccctgaagaagcgcggatacgatgtcaccaggaaccctcatctcaacaaggggatggcctttacccttgaagaaaggctgcagcttggaatccacggcctaatcccgccctgctttctgagccaggacgtccagctcctccgaatcatgagatattacgagcggcagcagagtgacctggacaagtacatcattctcatgacactccaagaccggaacgagaagctcttctaccgagtgctgacttcggatgtggagaagttcatgccaatcgtgtacacgcctaccgtggggctggcctgtcagcactatggcctgactttccgcaggccccgtggactgttcatcaccattcatgacaaaggtcatcttgcaacaatgctgaattcttggccagaagacaatattaaggccgtggtggtgactgatggggagcgcatcctgggcctgggagacctgggctgctacggcatgggcatccctgtgggcaagctggccctgtacacggcatgcggaggggtgaacccgcagcagtgcctccctgtgctgctggacgtcggcaccaacaatgaggagctgctcagagaccctctgtacatcggcctgaaacaccagcgcgtgcacgggaaggcatacgatgacttgctggatgagttcatgcaggctgtgacagacaagtttggaataaattgcctcatccaatttgaagacttcgccaatgccaatgccttccgcctgctcaacaaataccgtaacaagtactgcatgttcaatgatgacatccaaggcacagcctccgttgctgtggcagggatcttggctgctctgcgaatcaccaacaacaagctttccaatcacgtgtttgttttccaaggtgcaggcgaggcagctatgggcattgcccacctccttgtcatggccctagagaaagaaggtgtaccgaaggcagaggccacaagaaagatctggatggtggactctaaagggctcattgtcaaggggaggagccacctgaaccatgaaaaggagatgtttgcccaagaccatcctgaagtcaactccctggaggaggtggtgaggctggtgaagcccacagccatcataggtgttgctgccatcgcaggagccttcacggagcagattctgagggacatggcctccttccacgagcgccctatcatctttgccctgagcaaccccaccagcaaggccgagtgcacggctgagaagtgctaccgggtcaccgagggccgagggatttttgccagtggaagtccttttaagagtgtgactctggaagatggcaagaccttcattcctgggcagggaaacaatgcttacgtgttccccggggtggcactgggagtcatcgccggcgggatccggcacatcccagatgagatcttcctcctgacagcagagcaaattgcccaggaagtctctgagcagcatctgtcccaggggagactctatccaccactcagcaccatccgagacgtgtctttgagaattgccatcaaagttctcgactacgcgtacaaacacaacctggcttcctactacccagagcctaaggacaaggaggcttttgtaagatccctggtctacactccagactatgactcctttacactggacagctacacttggcccaaggaagccatgaatgttcagacggtctgaggcagtctcagaggctagtatggggctagatgaagcccagagtaacacccacaatataaatgggttccaaaatggcccaagtg

SEQ ID NO: 4 - protein, 604 aa, Homo sapiens
MGAALGTGTRLAPWPGRACGALPRWTPTAPAQGCHSKPGPARPVPLKKRGYDVTRNPHLNKGMAFTLEERLQLGIHGLIPPCFLSQDVQLLRIMRYYERQQSDLDKYIILMTLQDRNEKLFYRVLTSDVEKFMPIVYTPTVGLACQHYGLTFRRPRGLFITIHDKGHLATMLNSWPEDNIKAVVVTDGERILGLGDLGCYGMGIPVGKLALYTACGGVNPQQCLPVLLDVGTNNEELLRDPLYIGLKHQRVHGKAYDDLLDEFMQAVTDKFGINCLIQFEDFANANAFRLLNKYRNKYCMFNDDIQGTASVAVAGILAALRITNNKLSNHVFVFQGAGEAAMGIAHLLVMALEKEGVPKAEATRKIWMVDSKGLIVKGRSHLNHEKEMFAQDHPEVNSLEEVVRLVKPTAIIGVAAIAGAFTEQILRDMASFHERPIIFALSNPTSKAECTAEKCYRVTEGRGIFASGSPFKSVTLEDGKTFIPGQGNNAYVFPGVALGVIAGGIRHIPDEIFLLTAEQIAQEVSEQHLSQGRLYPPLSTIRDVSLRIAIKVLDYAYKHNLASYYPEPKDKEAFVRSLVYTPDYDSFTLDSYTWPKEAMNVQTV

SEQ ID NO: 5 - DNA, 785 bp, Homo sapiens
gaattgcaccatgccaaactacaaactcacttattttaatatgagggggagagcagaaattattcgttacatatttgcttatttggacatacagtatgaagaccacagaatagaacaagctgactggcctgaaatcaaatcaactctcccatttggaaaaatccccattttggaagttgatggacttactcttcaccagagcctagcaatagcaagatatttgaccaaaaacacagatttggctggaaacacagaaatggaacaatgtcatgttgatgctattgtggacactctggatgatttcatgtcatgttttccttgggcagagaaaaagcaagatgtgaaagagcagatgttcaatgagctgctcacgtataatgcgcctcatcttatgcaagacttggacacatatttaggggggagagaatggcttattggtaactctgtaacttgggcagacttctactgggagatttgcagtaccacacttttggtctttaagcctgacctgttagacaaccatccaaggctggtgactttacggaagaaagtccaagccattcctgccgtcgctaactggataaaacgaaggccccaaaccaaactctagctgatccatgttgccttcaagtttgtttttctcgggggcatctctctcatcagataagacagctacatcagcctgccagataatccacatgctccctccccagctccactaagattttcactttagccatattctgatttttaaaaaggaaaataaaaacaaatctttcttcagt

SEQ ID NO: 6 - protein, 199 aa, Homo sapiens
MPNYKLTYFNMRGRAEIIRYIFAYLDIQYEDHRIEQADWPEIKSTLPFGKIPILEVDGLTLHQSLAIARYLTKNTDLAGNTEMEQCHVDAIVDTLDDFMSCFPWAEKKQDVKEQMFNELLTYNAPHLMQDLDTYLGGREWLIGNSVTWADFYWEICSTTLLVFKPDLLDNHPRLVTLRKKVQAIPAVANWIKRRPQTKL

SEQ ID NO: 7 - DNA, 594 bp, Homo sapiens
atggcagaccagagacagcgctcactgtctacctctggggagtcattgtaccacgtccttgggttggacaagaacgcaacctcagatgacattaaaaagtcctatcggaagcttgccttgaaatatcaccccgacaagaaccccgacaacccggaggccgcggacaagtttaaggagatcaacaacgcgcacgccatcctcacggacgccacaaaaaggaacatctacgacaagtacggctcgctgggtctctacgtggccgagcagtttggggaagagaacgtgaacacctacttcgtgctgtccagctggtgggccaaggccctgtttgtcttctgcggcctcctcacgtgctgctactgctgctgctgtctgtgctgctgcttcaactgctgctgcgggaagtgtaagcccaaggcgcctgaaggcgaggagacggagttctacgtgtcccccgaggatctggaggcacagctgcagtctgacgagagggaggccacagacacgccgatcgtcatacagccggcatccgccaccgagaccacccagctcacagccgactcccaccccagctaccacactgacgggttcaac

SEQ ID NO: 8 - protein, 471 aa, Homo sapiens
MSRRVVRQSKFRHVFGQAAKADQAYEDIRVSKVTWDSSFCAVNPKFLAIIVEAGGGGAFIVLPLAKTGRVDKNYPLVTGHTAPVLDIDWCPHNDNVIASASDDTTIMVWQIPDYTPMRNITEPIITLEGHSKRVGILSWHPTARNVLLSAGGDNVIIIWNVGTGEVLLSLDDMHPDVIHSVCWNSNGSLLATTCKDKTLRIIDPRKGQVVAERFAAHEGMRPMRAVFTRQGHIFTTGFTRMSQRELGLWDPNNFEEPVALQEMDTSNGVLLPFYDPDSSIVYLCGKGDSSIRYFEITDEPPFVHYLNTFSSKEPQRGMGFMPKRGLDVSKCEIARFYKLHERKCEPIIMTVPRKSDLFQDDLYPDTPGPEPALEADEWLSGQDAEPVLISLRDGYVPPKHRELRVTKRNILDVRPPSGPRRSQSASDAPLSQHTLETLLEEIKALRERVQAQEQRITALENMLCELVDGTD

SEQ ID NO: 9 - DNA, 21 bp, Mus musculus
gggagaccttggctgtaatgg

SEQ ID NO: 10 - DNA, 17 bp, Mus musculus
acccctccacatgccgt

SEQ ID NO: 11 - DNA, 26 bp, Mus musculus
  misc_feature (1)..(1): FAM reporter dye
catccctgtgggtaaactggcccttt

SEQ ID NO: 12 - DNA, 21 bp, Mus musculus
caagccaactcttccatttgg

SEQ ID NO: 13 - DNA, 18 bp, Mus musculus
attgcgaggctctggtgg

SEQ ID NO: 14 - DNA, 30 bp, Mus musculus
  misc_feature (1)..(1): FAM reporter dye
atccctgttttggaggtggaaggacttaca

SEQ ID NO: 15 - DNA, 20 bp, Mus musculus
ggtcccagtgctgctgatgt

SEQ ID NO: 16 - DNA, 20 bp, Mus musculus
cctccatacaggcaggacca

SEQ ID NO: 17 - DNA, 25 bp, Mus musculus
  misc_feature (1)..(1): FAM reporter dye
tctctgtgccccatgtgctgtctcc

SEQ ID NO: 18 - DNA, 23 bp, Mus musculus
acctcacaaggagcacctgtaca

SEQ ID NO: 19 - DNA, 20 bp, Mus musculus
taatgctggtcttgcccaca

SEQ ID NO: 20 - DNA, 21 bp, Mus musculus
  misc_feature (1)..(1): FAM reporter dye
tgctggtgatcggcgacctgg

SEQ ID NO: 21 - DNA, 20 bp, Mus musculus
ggcacagctgcagtctgatg

SEQ ID NO: 22 - DNA, 20 bp, Mus musculus
tggcagatgctggctgtatg

SEQ ID NO: 23 - DNA, 26 bp, Mus musculus
  misc_feature (1)..(1): FAM reporter dye
aagggaggctacagacacaccgatcg

SEQ ID NO: 24 - DNA, 18 bp, Mus musculus
cgtcgccagaccctgatt

SEQ ID NO: 25 - DNA, 15 bp, Mus musculus
caaacggcggctccc

SEQ ID NO: 26 - DNA, 27 bp, Mus musculus
  misc_feature (1)..(1): FAM reporter dye
cacagtccgagacgtcaaactcctggt

SEQ ID NO: 27 - DNA, 19 bp, Mus musculus
tcgacatggactcccggat

SEQ ID NO: 28 - DNA, 22 bp, Mus musculus
caggagtagtgtgctcttcccc

SEQ ID NO: 29 - DNA, 22 bp, Mus musculus
  misc_feature (1)..(1): FAM reporter dye
tgcatcgtgggtcccaatggtg

SEQ ID NO: 30 - DNA, 20 bp, Mus musculus
agggaccatctcctcgacct

SEQ ID NO: 31 - DNA, 22 bp, Mus musculus
cccatctctgctgctttttctg

SEQ ID NO: 32 - DNA, 20 bp, Mus musculus
  misc_feature (1)..(1): FAM reporter dye
cccaacccactgccccctca

SEQ ID NO: 33 - DNA, 15 bp, Mus musculus
ccgcgcactcccagg

SEQ ID NO: 34 - DNA, 24 bp, Mus musculus
caaatctgacatggaatgtctcca

SEQ ID NO: 35 - DNA, 24 bp, Mus musculus
  misc_feature (1)..(1): FAM reporter dye
agggcagagagggagacactgcca

SEQ ID NO: 36 - DNA, 17 bp, Mus musculus
tgagacccatgcgggct

SEQ ID NO: 37 - DNA, 17 bp, Mus musculus
tcgggtgaatcccgtgg

SEQ ID NO: 38 - DNA, 26 bp, Mus musculus
  misc_feature (1)..(1): FAM reporter dye
tcttcacgcggctgggtcatatcttc

SEQ ID NO: 39 - DNA, 20 bp, Mus musculus
ggcttggtgttcagggtctc

SEQ ID NO: 40 - DNA, 21 bp, Mus musculus
actgcaatgcctccagagtca

SEQ ID NO: 41 - DNA, 24 bp, Mus musculus
  misc_feature (1)..(1): FAM reporter dye
cctgcactcacctcttgctgcctg

SEQ ID NO: 42 - DNA, 1949 bp, Homo sapiens
gctgagcatcgccagggcgggcggcagggcgcggcctctccgccgggtgtacctcctgtcgcggcgcgagacctctggtgaaagaaaagatgttgtcccggttaagagtagtttccaccacttgtactttggcatgtcgacatttgcacataaaagaaaaaggcaagccacttatgctgaacccaagaacaaacaagggaatggcatttactttacaagaacgacaaatgcttggtcttcaaggacttctacctcccaaaatagagacacaagatattcaagccttacgatttcatagaaacttgaagaaaatgactagccctttggaaaaatatatctacataatgggaatacaagaaagaaatgagaaattgttttatagaatactgcaagatgacattgagagtttaatgccaattgtatatacaccgacggttggtcttgcctgctcccagtatggacacatctttagaagacctaagggattatttatttcgatctcagacagaggtcatgttagatcaattgtggataactggccagaaaatcatgttaaggctgttgtagtgactgatggagagagaattctgggtcttggagatctgggtgtctatggaatgggaattccagtaggaaaactttgtttgtatacagcttgtgcaggaatacggcctgatagatgcctgccagtgtgtattgatgtgggaactgataatatcgcactcttaaaagacccattttacatgggcttgtaccagaaacgagatcgcacacaacagtatgatgacctgattgatgagtttatgaaagctattactgacagatatggccggaacacactcattcagttcgaagactttggaaatcataatgcattcaggttcttgagaaagtaccgagaaaaatattgtactttcaatgatgatattcaagggacagctgcagtagctctagcaggtcttcttgcagcacaaaaagttattagtaaaccaatctccgaacacaaaatcttattccttggagcaggagaggctgctcttggaattgcaaatcttatagttatgtctatggtagaaaatggcctgtcagaacaagaggcacaaaagaaaatctggatgtttgacaagtatggtttattagttaagggacggaaagcaaaaatagatagttatcaggaaccatttactcactcagccccagagagcatacctgatacttttgaagatgcagtgaatatactgaagccttcaactattattggagttgcaggtgctggccgtcttttcactcctgatgtaatcagagccatggcctctatcaatgaaaggcctgtaatatttgcattaagtaatcctacagcacaggcagagtgcacggctgaagaagcatatacacttacagagggcaggtgtttgtttgccagtggcagtccatttgggccagtgaaacttacagatgggcgagtctttacaccaggtcaaggaaacaatgtttatatttttccaggtgtggctttagctgttattctctgtaacacccggcatattagtgacagtgttttcctagaagctgcaaaggccctgacaagccaattgacagatgaagagctagcccaagggagactttacccaccgcttgctaatattcaggaagtttctattaacattgctattaaagttacagaatacctatatgctaataaaatggctttccgatacccagaacctgaagacaaggccaaatatgttaaagaaagaacatggcggagtgaatatgattccctgctgccagatgtgtatgaatggccagaatctgcatcaagccctcctgtgataacagaatagaagcactcccctgataaatactttctgtgctccagggaaccccttttttcagacaagaagagataatgtcttcagttttaaaaaaaaaaaaaaaaaaaaaaaaaa

SEQ ID NO: 43 - protein, 584 aa, Homo sapiens
MLSRLRVVSTTCTLACRHLHIKEKGKPLMLNPRTNKGMAFTLQERQMLGLQGLLPPKIETQDIQALRFHRNLKKMTSPLEKYIYIMGIQERNEKLFYRILQDDIESLMPIVYTPTVGLACSQYGHIFRRPKGLFISISDRGHVRSIVDNWPENHVKAVVVTDGERILGLGDLGVYGMGIPVGKLCLYTACAGIRPDRCLPVCIDVGTDNIALLKDPFYMGLYQKRDRTQQYDDLIDEFMKAITDRYGRNTLIQFEDFGNHNAFRFLRKYREKYCTFNDDIQGTAAVALAGLLAAQKVISKPISEHKILFLGAGEAALGIANLIVMSMVENGLSEQEAQKKIWMFDKYGLLVKGRKAKIDSYQEPFTHSAPESIPDTFEDAVNILKPSTIIGVAGAGRLFTPDVIRAMASINERPVIFALSNPTAQAECTAEEAYTLTEGRCLFASGSPFGPVKLTDGRVFTPGQGNNVYIFPGVALAVILCNTRHISDSVFLEAAKALTSQLTDEELAQGRLYPPLANIQEVSINIAIKVTEYLYANKMAFRYPEPEDKAKYVKERTWRSEYDSLLPDVYEWPESASSPPVITE

SEQ ID NO: 44 - protein, 572 aa, Homo sapiens
MEPEAPRRRHTHQRGYLLTRNPHLNKDLAFTLEERQQLNIHGLLPPSFNSQEIQVLRVVKNFEHLNSDFDRYLLLMDLQDRNEKLFYRVLTSDIEKFMPIVYTPTVGLACQQYSLVFRKPRGLFITIHDRGHIASVLNAWPEDVIKAIVVTDGERILGLGDLGCNGMGIPVGKLALYTACGGMNPQECLPVILDVGTENEELLKDPLYIGLRQRRVRGSEYDDFLDEFMEAVSSKYGMNCLIQFEDFANVNAFRLLNKYRNQYCTFNDDIQGTASVAVAGLLAALRITKNKLSDQTILFQGAGEAALGIAHLIVMALEKEGLPKEKAIKKIWLVDSKGLIVKGRASLTQEKEKFAHEHEEMKNLEAIVQEIKPTALIGVAAIGGAFSEQILKDMAAFNERPIIFALSNPTSKAECSAEQCYKITKGRAIFASGSPFDPVTLPDGRTLFPGQGNNSYVFPGVALGVVACGLRHIPDKVFLTTAEVISQQVSDKHLQEGRLYPPLNTIRDVSLKIAVKIVQDAYKEKMATVYPEPQNKEEFVSSQMYSTNYDQILPDCYPWPAEVQKIQTKVNQ

SEQ ID NO: 45 - protein, 763 aa, Drosophila melanogaster
MGNSSSICADRNVITNFDENGTPVYPTANNSQSPSSYSRGKERELGCYTKRNSNSNNNNSHERESQSCCSSRVCKNHTTTTTTTLEYELSNFAKLTTRITTQSAAEVDTSPHTDTETHRDRDSNPGNIALATDLELPKGLPLSLSSRHHWNQLQSSLHALHHQQQQQQQQLRSYSSTSETNLEDKMSKPDSKLDKYAQRDRLGLWGTGDNEVVGSLSGFTRLLDKRYSKGLAFTHEERQQLGIHGMLPYVVREPSEQVEHCRALLARLDQDLDKYMYLISLSERNERLFYNVLSSDIAYMMPLVYTPTVGLACQRYSLIHQNAKGMFISIKDKGHIYDVLKNWPETDVRAIVVTDGERILGLGDLGANGMGIPVGKLSLYTALAGIKPSQCLPITLDVGTNTESILEDPLYIGLRERRATGDLYDEFIDEFMHACVRRFGQNCLIQFEDFANANAFRLLSKYRDSFCTFNDDIQGTASVAVAGLLASLKIKKTQLKDNTLLFLGAGEAALGIANLCLMAMKVEGLTEEEAKARIWMVDSRGVITRDRPKGGLTEHKLHFAQLHEPIDTLAEAVRKVRPNVLIGAAAQGGAFNQEILELMADINETPIIFALSNPTSKAECTAEEAYTYTKGRCIFASGSPFAPVTYNNKKFYPGQGNNSYIFPGVALGVLCAGMLNIPEQVFLVAAERLAELVSKDDLAKGSLYPPLSSIVSCSMAIAERIVEYAYKNGLATVRPEPVNKLAFIKAQMYDLDYPRSVPATYKM

SEQ ID NO: 46 - protein, 223 aa, Drosophila melanogaster
MDQEMEPAIMTSTSDKREHLYKILVIGELGTGKTSFIKRYVHQFFSQNYRATIGVDFALKVLQWDANTIVRLQLWDIAGQERFGNMTRVYYKEAVGAFIVFDVTRSGTFDCVSKWKEDLDSKVQLPDGSPIPCILLANKCDQEKQGIITQPEKMDEYVRENGFAGWFETSAKENINIDEAARALVNKILINDKLISADLADGDKFNLSAADATGSDAKNKCSC

SEQ ID NO: 47 - protein, 225 aa, Homo sapiens
MAGGGAGDPGLGAAAAPAPETREHLFKVLVIGELGVGKTSIIKRYVHQLFSQHYRATIGVDFALKVLNWDSRTLVRLQLWDIAGQERFGNMTRVYYKEAVGAFVVFDISRSSTFEAVLKWKSDLDSKVHLPNGSPIPAVLLANKCDQNKDSSQSPSQVDQFCKEHGFAGWFETSAKDNINIEEAARFLVEKILVNHQSFPNEENDVDKIKLDQETLRAENKSQCC

SEQ ID NO: 48 - protein, 211 aa, Homo sapiens
MQAPHKEHLYKLLVIGDLGVGKTSIIKRYVHQNFSSHYRATIGVDFALKVLHWDPETVVRLQLWDIAGQERFGNMTRVYYREAMGAFIVFDVTRPATFEAVAKWKNDLDSKLSLPNGKPVSVVLLANKCDQGKDVLMNNGLKMDQFCKEHGFVGWFETSAKENINIDEASRCLVKHILANECDLMESIEPDVVKPHLTSTKVASCSGCAKS

SEQ ID NO: 49 - protein, 203 aa, Homo sapiens
MGSRDHLFKVLVVGDAAVGKTSLVQRYSQDSFSKHYKSTVGVDFALKVLQWSDYEIVRLQLWDIAGQERFTSMTRLYYRDASACVIMFDVTNATTFSNSQRWKQDLDSKLTLPNGEPVPCLLLANKCDLSPWAVSRDQIDRFSKENGFTGWTETSVKENKNINEAMRVLIEKMMRNSTEDIMSLSTQGDYINLQTKSSSWSCC

SEQ ID NO: 50 - protein, 199 aa, Homo sapiens
MACNIPNQRQRTLSTTGEALYEILGLHKGASNEEIKKTYRKLALKHHPDKNPDDPAATEKFKEINNAHAILTDISKRSIYDKYGSLGLYVAEQFGDENVNTYFMLSSWWAKALFVIVGLLTGCYFCCCLCCCCNCCCGHCRPESSVPEEDFYVSPEDLEEQIKSDMEKDVDFPVFLQPTNANEKTQLIKEGSRSYCTDS

SEQ ID NO: 51 - protein, 198 aa, Homo sapiens
MADQRQRSLSTSGESLYHVLGLDKNATSDDIKKSYRKLALKYHPDKNPDNPEAADKFKEINNAHAILTDATKRNIYDKYGSLGLYVAEQFGEENVNTYFVLSSWWAKALFVFCGLLTCCYCCCCLCCCFNCCCGKCKPKAPEGEETEFYVSPEDLEAQLQSDEREATDTPIVIQPASATETTQLTADSHPSYHTDGFN

SEQ ID NO: 52 - protein, 220 aa, Drosophila melanogaster
MSAPGMDKRKLSTSGDSLYEILGLPKTATGDDIKKTYRKLALKYHPDKNPDNVDAADKFKEVNRAHSILSDQTKRNIYDNYGSLGLYIAEQFGEENVNAYFVVTSPAVKAVVICCAVITGCCCCCCCCCCCNFCCGKFKPPVNESHDQYSHLNEDVDLDDVNLGAGGAPVTSQPREQAGGQPVFAMPPPSGAVGVNPFTGAPVAANENTSLNTTEQTTYT

SEQ ID NO: 53 - protein, 189 aa, Homo sapiens
MSTVKEAAHRLSKSEMSLYAVLDLKKGASPEDFKKSYSHSALLPHPPFEYHLGRKLALRYHPDKNPGNAQAAEIFKEINAAHAILSDSKKRKIYDQHGSLGIYLYDHFGEEGVRYYFILNSCWFKTLVILCTLLTCCCFCCCCCFCCGALKPPPEQDSGRKYQQNVQSQPPRSGAKCDFRSEENSEDDF

SEQ ID NO: 54 - protein, 1162 aa, Homo sapiens
MEPEEERIRYSQRLRGTMRRRYEDDGISDDEIEGKRTFDLEEKLHTNKYNANFVTFMEGKDFNVEYIQRGGLRDPLIFKNSDGLGIKMPDPDFTVNDVKMCVGSRRMVDVMDVNTQKGIEMTMAQWTRYYETPEEEREKLYNVISLEFSHTRLENMVQRPSTVDFIDWVDNMWPRHLKESQTESTNAILEMQYPKVQKYCLMSVRGCYTDFHVDFGGTSVWYHIHQGGKVFWLIPPTAHNLELYENWLLSGKQGDIFLGDRVSDCQRIELKQGYTFVIPSGWIHAVYTPTDTLVFGGNFLHSFNIPMQLKIYNIEDRTRVPNKFRYPFYYEMCWYVLERYVYCITNRSHLTKEFQKESLSMDLELNGLESGNGDEEAVDREPRRLSSRRSVLTSPVANGVNLDYDGLGKTCRSLPSLKKTLAGDSSSDCSRGSHNGQVWDPQCAPRKDRQVHLTHFELEGLRCLVDKLESLPLHKKCVPTGIEDEDALIADVKILLEELANSDPKLALTGVPIVQWPKRDKLKFPTRPKVRVPTIPITKPHTMKPAPRLTPVRPAAASPIVSGARRRRVRCRKCKACVQGECGVCHYCRDMKKFGGPGRMKQSCVLRQCLAPRLPHSVTCSLCGEVDQNEETQDFEKKLMECCICNEIVHPGCLQMDGEGLLNEELPNCWECPKCYQEDSSEKAQKRKMEESDEEAVQAKVLRPLRSCDEPLTPPPHSPTSMLQLIHDPVSPRGMVTRSSPGAGPSDHHSASRDERFKRRQLLRLQATERTMVREKENNPSGKKELSEVEKAKIRGSYLTVTLQRPTKELHGTSIVPKLQAITASSANLRHSPRVLVQHCPARTPQRGDEEGLGGEEEEEEEEEEEDDSAEEGGAARLNGRGSWAQDGDESWMQREVWMSVFRYLSRRELCECMRVCKTWYKWCCDKRLWTKIDLSRCKAIVPQALSGIIKRQPVSLDLSWTNISKKQLTWLVNRLPGLKDLLLAGCSWSAVSALSTSSCPLLRTLDLRWAVGIKDPQIRDLLTPPADKPGQDNRSKLRNMTDFRLAGLDITDATLRLIIRHMPLLSRLDLSHCSHLTDQSSNLLTAVGSSTRYSLTELNMAGCNKLTDQTLIYLRRIANVTLIDLRGCKQITRKACEHFISDLSINSLYCLSDEKLIQKIS

SEQ ID NO: 55 - protein, 1051 aa, Homo sapiens
KLLVGSRRLVDVMDVNTQKGTEMSMSQFVRYYETPEAQRDKLYNVISLEFSHTKLEHLVKRPTVVDLVDWVDNMWPQHLKEKQTEATNAIAEMKYPKVKKYCLMSVKGCFTDFHIDFGGTSVWYHVFRGGKIFWLIPPTLHNLALYEEWVLSGKQSDIFLGDRVERCQRIELKQGYTFFIPSGWIHAVYTPVDSLVFGGNILHSFNVPMQLRIYEIEDRTRVQPKFRYPFYYEMCWYVLERYVYCVTQRSHLTQEYQRESMLIDAPRKPSIDGFSSDSWLEMEEEACDQQPQEEEEKDEEGEGRDRAPKPPTDGSTSPTSTPSEDQEALGKKPKAPALRFLKRTLSNESEESVKSATLAVDYPKTPTGSPATEVSAKWTHLTEFELKGLKALVEKLESLPENKKCVPEGIEDPQALLEGVKNVLKEHADDDPSLAITGVPVVTWPKKTPKNRAVGRPKGKLGPASAVKLAANRTTAGARRRRTRCRKCEACLRTECGECHFCKDMKKFGGPGRMKQSCIMRQCIAPVLPHTAVCLVCGEAGKEDTVEEEEGKFNLMLMECSICNEIIHPGCLKIKESEGVVNDELPNCWECPKCNHAGKTGKQKRGPGFKYASNLPGSLLKEQKMNRDNKEGQEPAKRRSECEEAPRRRSDEHSKKVPPDGLLRRKSDDVHLRKKRKYEKPQELSGRKRASSLQTSPGSSSHLSPRPPLGSSLSPWWRSSLTYFQQQLKPGKEDKLFRKKRRSWKNAEDRMALANKPLRRFKQEPEDELPEAPPKTRESDHSRSSSPTAGPSTEGAEGPEEKKKVKMRRKRRLPNKELSRELSKELNHEIQRTENSLANENQQPIKSEPESEGEEPKRPPGICERPHRFSKGLNGTPRELRHQLGPSLRSPPRVISRPPPSVSPPKCIQMERHVIRPPPISPPPDSLPLDDGAAHVMHREVWMAVFSYLSHQDLCVCMRVCRTWNRWCCDKRLWTRIDLNHCKSITPLMLSGIIRRQPVSLDLSWTNISKKQLSWLINRLPGLRDLVLSGCSWIAVSALCSSSCPLLRTLD

SEQ ID NO: 56 - protein, 91 aa, Mus musculus
HASPDPDFTVRDVKLLVGSRRLVDVMDVNTQKGTEMSMSQLCATTRHQRHSGINCTTSSASSSAIRSWSIWSSVPLWWTWSTGWTTCGLSI

SEQ ID NO: 57 - protein, 1368 aa, Drosophila melanogaster
MSTAVETGSSPAKSNSNNSSSGGNNNNGNGNLSPNAKGVQRRQLRERKQRKLYLEEWSLGDEDGEGTRGFSVAEKLESSKFAQAGMVREMRGCDLTVAFLQQHGFNIPLLFRDKAGLGLRMPDPQEFTVNDVRLCVGSRRLLDVMDVNTQKNLQMTMKEWQQYYDSPQKDRLLNVISLEFSHTRLDRFVQSPEIVRQIDWVDVVWPKQLKDAQREGTNLLGGMMYPKVQKYCLMSVKNCYTDFHIDFGGTSVWYHILRGSKVFWLIPPTDRNLQLYEKWVLSGKQADIFFGDTVEKCARVYLTAGNTFFIPTGWIHAVYTPTQSLVFGGNFLHSFGIVKQLKTASVEDSTKVPQKFRYPFFTEMLWYVLARYVHTLLGHSHLEGEASLSEDEMAARPHTHLTHHELFGLKEIVMYLYDLPPQKKNVPSLVLDPVALIKDVRSLVERHCKDQQDLAITGVSVLKSPPGSQPPFLLYDRTRVKQEIKQEIARKNAEVIREQQQLEAGRAREAESDTSQSTGVGSVIGMGAGVEYSNGVMKKEQLENGSGVTVGGHGSQPEATFALPTDTLKYRPPKKMHLATALVAAAASSSSGGGGPVAGVGGSAVVGSSHSPTGGGVGPVTGAGGAISVIATSSSYIEGGQVGGILNMDNCHSPEGGGAKLSPNLTGTGQPRRRRTRCKNCAACQRSDCGTCPFCMDMVKFGGPGRAKQTCMMRQCLSPMLPVTAQCVYCHLDGWRQTPVSPQTKQLASADGPSALMECSVCYEIAHPDCALSQLDGTEDAADAKGIVNEDLPNSWECPSCCRSGKNYDYKVRLPSTHVSLVLVQLTNPTFIVQPRHFRARQKSSEVRRVSVSHGQGGAEGHADGNTLLPPPVGQYNDFVFTSESEMESGTVSGHMTHWKHGMKRHHQLEVKTERNNSCDTPSPGISPNAIGGDSKVGKRRKSDDGTSVSSSMHESNDAPCGSSAEGAGGAGNANVSTNQWSGSGGGGGSRKKNSIRSQLAQQMLNSSTRVLKKPQYVVRPASGTGSSSSSGNGGSASATNGISNGSNQSGANSCGAGNGERGTNNGGLSGSNGLGNQHYSSSQNLALDPTVLKIIFRYLPQDTLVTCCSVCKVWSNAAVDPDLWKKMNCSEHKMSASLLTAIVRRQPEHLILDWTQIAKRQLAWLVARLPALKNLSLQNCPIQAVLALHTCLCPPLQTLDLSFVRGLNDAAIRDILSPPKDSRPGLSDSKTRLRDLKVMKLAGTDISDVAVRYITQSLPYLRHLDLSSCQRITDAGVAQIGTSTTATARLTELNLSACRLVSENALEHLAKCEGLIWLDLRHVPQVSTQSVIRFASNSKHDLCVRDIKLVERRRRNSTTANRSWHHD

SEQ ID NO: 58 - protein, 1162 aa, Homo sapiens; identical to SEQ ID NO: 54

SEQ ID NO: 59 - protein, 1336 aa, Homo sapiens
MAGPQMGGSAEDHPPRKRHAAEKQKKKTVIYTKCFEFESATQRPIDRQRYDENEDLSDVEEIVSVRGFSLEEKLRSQLYQGDFVHAMEGKDFNYEYVQREALRVPLIFREKDGLGIKMPDPDFTVRDVKLLVGSRRLVDVMDVNTQKGTEMSMSQFVRYYETPEAQRDKLYNVISLEFSHTKLEHLVKRPTVVDLVDWVDNMWPQHLKEKQTEATNAIAEMKYPKVKKYCLMSVKGCFTDFHIDFGGTSVWYHVFRGGKIFWLIPPTLHNLALYEEWVLSGKQSDIFLGDRVERCQRIELKQGYTFFIPSGWIHAVYTPVDSLVFGGNILHSFNVPMQLRIYEIEDRTRVQPKFRYPFYYEMCWYVLERYVYCVTQRSHLTQEYQRESMLIDAPRKPSIDGFSSDSWLEMEEEACDQQPQEEEEKDEEGEGRDRAPKPPTDGSTSPTSTPSEDQEALGKKPKAPALRFLKRTLSNESEESVKSTTLAVDYPKTPTGSPATEVSAKWTHLTEFELKGLKALVEKLESLPENKKCVPEGIEDPQALLEGVKNVLKEHADDDPSLAITGVPVVTWPKKTPKNRAVGRPKGKLGPASAVKLAANRTTAGARRRRTRCRKCEACLRTECGECHFCKDMKKFGGPGRMKQSCIMRQCIAPVLPHTAVCLVCGEAGKEDTVEEEEGKFNLMLMECSICNEIIHPGCLKIKESEGVVNDELPNCWECPKCNHAGKTGKQKRGPGFKYASNLPGSLLKEQKMNRDNKEGQEPAKRRSECEEAPRRRSDEHSKKVPPDGLLRRKSDDVHLRKKRKYEKPQELSGRKRASSLQTSPGSSSHLSPRPPLGSSLSPWWRSSLTYFQQQLKPGKEDKLFRKKRRSWKNAEDRMALANKPLRRFKQEPEDELPEAPPKTRESDHSRSSSPTAGPSTEGAEGPEEKKKVKMRRKRRLPNKELSRELSKELNHEIQRTENSLANENQQPIKSEPESEGEEPKRPPGICERPHRFSKGLNGTPRELRHQLGPSLRSPPRVISRPPPSVSPPKCIQMERHVIRPPPISPPPDSLPLDDGAAHVMHREVWMAVFSYLSHQDLCVCMRVCRTWNRWCCDKRLWTRIDLNHCKSITPLMLSGIIRRQPVSLDLSWTNISKKQLSWLINRLPGLRDLVLSGCSWIAVSALCSSSCPLLRTLDVQWVEGLKDAQMRDLLSPPTDNRPGQMDNRSKLRNIVELRLAGLDITDASLRLIIRHMPLLSKLHLSYCNHVTDQSINLLTAVGTTTRDSLTEINLSDCNKVTDQCLSFFKRCGNICHIDLRYCKQVTKEGCEQFIAEMSVSVQFGQVEEKLLQKLS

SEQ ID NO: 60 - protein, 272 aa, Homo sapiens
ADEIIIKMHGSQLTQRYLEKHGFDVPIMVPKLDDLGLRLPSPTFSVMDVERYVGGDKVIDVIDVARQADSKMTLHNYVKYFMNPNRPKVLNVISLEFSDTKMSELVEVPDIAKKLSWVENYWPDDSVFPKPFVQKYCLMGVQDSYTDFHIDFGGTSVWYHVLWGEKIFYLIKPTDENLARYESWSSSVTQSEVFFGDKVDKCYKCVVKQGHTLFVPTGWIHAVLTSQDCMAFGGNFLHNLNIGMQLRCYEMEKRLKTPDLFKFPFFEAICWF

SEQ ID NO: 61 - protein, 1100 aa, Homo sapiens
MATVPVYCVCRLPYDVTRFMIECDACKDWFHGSCVGVEEEEAPDIDIYHCPNCEKTHGKSTLKKKRTWHKHGPGQAPDVKPVQNGSQLFIKELRSRTFPSAEDVVARVPGSQLTLGYMEEHGFTEPILVPKKDGLGLAVPAPTFYVSDVENYVGPERSVDVTDVTKQKDCKMKLKEFVDYYYSTNRKRVLNVTNLEFSDTRMSSFVEPPDIVKKLSWVENYWPDDALLAKPKVTKYCLICVKDSYTDFHIDSGGASAWYHVLKGEKTFYLIRPASANISLYERWRSASNHSEMFFADQVDKCYKCIVKQGQTLFIPSGWIYATLTPVDCLAFAGHFLHSLSVEMQMRAYEVERRLKLGSLTQFPNFETACWYMGKHLLEAFKGSHKSGKQLPPHLVQGAKILNGAFRSWTKKQALAEHEDELPEHFKPSQLIKDLAKEIRLSENASKAVRPEVNTVASSDEVCDGDREKEEPPSPIEATPPQSLLEKVSKKKTPKTVKMPKPSKIPKPPKPPKPPRPPKTLKLKDGGKKKGKKSRESASPTIPNLDLLEAHTKEALTKMEPPKKGKATKSVLSVPNKDVVHMQNDVERLEIREQTKSKSEAKWKYKNSKPDSLLKMEEEQKLEKSPLAGNKDNKFSFSFSNKKLLGSKALRPPTSPGVFGALQNFKEDKPKPVRDEYEYVSDDGELKIDEFPIRRKKNAPKRDLSFLLDKKAVLPTPVTKPKLDSAAYKSDDSSDEGSLHIDTDTKPGRNARVKKESGSSAAGILDLLQASEEVGALEYNPSSQPPASPSTQEAIQGMLSMANLQASDSCLQTTWGAGQAKGSSLAAHGARKNGGGSGKSAGKRLLKRAAKNSVDLDDYEEEQDHLDACFKDSDYVYPSLESDEDNPIFKSRSKKRKGSDDAPYSPTARVGPSVPRQDRPVREGTRVASIETGLAAAAAKLSQQEEQKSKKKKSAKRKLTPNTTSPSTSTSISAGTTSTSTTPASTTPASTTPASTTPASTSTASSQASQEGSSPEPPPESHSSSLADHEYTAAGTFTGAQAGRASQPMAPGVFLTQRRPSASSPNNTAAKGKRTKKGMATAKQRLGKILKIHRNGKLLL

SEQ ID NO: 62 - protein, 489 aa, Homo sapiens
MSFRKVVRQSKFRHVFGQPVKNDQCYEDIRVSRVTWDSTFCAVNPKFLAVIVEASGGGAFLVLPLSKTGRIDKAYPTVCGHTGPVLDIDWCPHNDEVIASGSEDCTVMVWQIPENGLTSPLTEPVVVLEGHTKRVGIIAWHPTARNVLLSAGCDNVVLIWNVGTAEELYRLDSLHPDLIYNVSWNHNGSLFCSACKDKSVRIIDPRRGTLVAEREKAHEGARPMRAIFLADGKVFTTGFSRMSERQLALWDPENLEEPMALQELDSSNGALLPFYDPDTSVVYVCGKGDSSIRYFEITEEPPYIHFLNTFTSKEPQRGMGSMPKRGLEVSKCEIARFYKLHERKCEPIVMTVPRKSDLFQDDLYPDTAGPEAALEAEEWVSGRDADPILISLREAYVPSKQRDLKISRRNVLSDSRPAMAPGSSHLGAPASTTTAADATPSGSLARAGEAGKLEEVMQELRALRALVKEQGDRICRLEEQLGRMENGDA

SEQ ID NO: 63 - protein, 474 aa, Homo sapiens
MRRVVRQSKFRHVFGQAVKNDQCYDDIRVSRVTWDSSFCAVNPRFVAIIIEASGGGAFLVLPLHKTGRIDKSYPTVCGHTGPVLDIDWCPHNDQVIASGSEDCTVMVWQIPENGLTLSLTEPVVILEGHSKRVGIVAWHPTARNVLLSAGCDNAIIIWNVGTGEALINLDDMHSDMIYNVSWNRNGSLICTASKDKKVRVIDPRKQEIVAEKEKAHEGARPMRAIFLADGNVFTTGFSRMSERQLALWNPKNMQEPIALHEMDTSNGVLLPFYDPDTSIIYLCGKGDSSIRYFEITDESPYVHYLNTFSSKEPQRGMGYMPKRGLDVNKCEIARFFKLHERKCEPIIMTVPRKSDLFQDDLYPDTAGPEAALEAEEWFEGKNADPILISLKHGYIPGKNRDLKVVKKNILDSKPTANKKCDLISIPKKTTDTASVQNEAKLDEILKEIKSIKDTICNQDERISKLEQQMAKIAA

SEQ ID NO: 64 - protein, 528 aa, Drosophila melanogaster
MSFRVVRSSKFRHVYGQALKREQCYDNIRVSKSSWDSTFCAVNPKFLAIIVESAGGGAFIVLPHNKVGRIAADHPLVGGHKGPVLDIAWCPHNDNVIASGSEDCVVKVWQIPDGGLSRTLTEPVVDLVFHQRRVGLVLWHPSALNVLLTAGSDNQVVIWNVGTGEILVHIDSHPDIVYSACFNWDGSKLVTTCKDKKIRIYDPRTAELESEAMCHEGSKATRAIFLRHGLIFTTGFNRSSERQYSLRAPDALNEPIVMVELDTSNGVMFPLYDADTNMIYLCGKGDSVIRYFEVTPEPPFVHYINTFQTTEPQRGIGLMPKRGCDVTTCEVAKFYRMNNNGLCQVISMTVPRKSDLFQEDLYPDTLAEDAAITAEEWIDGKDADPITFSLKGGYVSSSVNKSLPAKKAGNILNKPRGDSASSGATSSSAGGGNFASGNNNEASEGGPPAAVLSEKDLRTIQDEIRKLKAIIVKQENRIRALEAKEDARNKNGSDAAPASAGAATSDGKASESANDHASTSAGTSKDED

SEQ ID NO: 65 - protein, 461 aa, Homo sapiens
MSRQVVRSSKFRHVFGQPAKADQCYEDVRVSQTTWDSGFCAVNPKFVALICEASGGGAFLVLPLGKTGRVDKNAPTVCGHTAPVLDIAWCPHNDNVIASGSEDCTVMVWEIPDGGLMLPLREPVVTLEGHTKRVGIVAWHTTAQNVLLSAGCDNVIMVWDVGTGAAMLTLGPEVHPDTIYSVDWSRDGGLICTSCRDKRVRIIEPRKGTVVAEKDRPHEGTRPVRAVFVSEGKILTTGFSRMSERQVALWDTKHLEEPLSLQELDTSSGVLLPFFDPDTNIVYLCGKGDSSIRYFEITSEAPFLHYLSMFSSKESQRGMGYMPKRGLEVNKCEIARFYKLHERRCEPIAMTVPRKSDLFQEDLYPPTAGPDPALTAEEWLGGRDAGPLLISLKDGYVPPKSRELRVNRGLDTGRRRAAPEASGTPSSDAVSRLEEEMRKLQATVQELQKRLDRLEETVQAK

SEQ ID NO: 66 - protein, 477 aa, Homo sapiens
MSWRPQYRSSKFRNVYGKVANREHCFDGIPITKNVHDNHFCAVNTRFLAIVTESAGGGSFLVIPLEQTGRIEPNYPKVCGHQGNVLDIKWNPFIDNIIASCSEDTSVRIWEIPEGGLKRNMTAALLELHGHSRRVGLVEWHPTTNNILFSAGYDYKVLIWNLDVGEPVKMIDCHTDVILCMSFNTDGSLLTTTCKDKKLRVIEPRSGRVLQEANCKNHRVNRVVFLGNMKRLLTTGVSRWNTRQIALWDQEDLSMPLIEEEIDGLSGLLFPFYDADTHMLYLAGKGDGNIRYYEISTEKPYLSYLMEFRSPAPQKGLGVMPKHGLDVSACEVFRFYKLVTLKGLIEPISMIVPRRSDSYQEDLYPDTLAEDDAITAEEWIDGKDADPITFSLKGGEGYKKSSKMVFKAPIKEKKSVVVNGIDLLENVPPRTENELLRMFFRQQDEIRRLKEELAQKDIRIRQLQLELKNLRNSPKNC

SEQ ID NO: 67 - protein, 567 aa, Homo sapiens
MSWHPQYRSSKFRHVFGKPASKENCYDSVPITRSVHDNHFCAVNPHFIAVVTECAGGGAFLVIPLHQTGKLDPHYPKVCGHRGNVLDVKWNPFDDFEIASCSEDATIKIWSIPKQLLTRNLTAYRKELVGHARRVGLVEWHPTAANILFSAGYDYKVMIWNLDTKESVITSPMSTISCHQDVILSMSFNTNGSLLATTCKDRKIRVIDPRAGTVLQEASYKGHRASKVLFLGNLKKLMSTGTSRWNNRQVALWDQDNLSVPLMEEDLDGSSGVLFPFYDADTSMLYVVGKGDGNIRYYEVSADKPHLSYLTEYRSYNPQKGIGVMPKRGLDVSSCEIFRFYKLITTKSLIEPISMIVPRRSDSYQEDIYPMTPGTEPALTPDEWLGGINRDPVLMSLKVPRRSESYQEDIYPPTAGAQPSLTAQEWLSGMNRDPILVSLRPGSELLRPHPLPAERPIFNSMAPASPRLLNQTEKLAAEDGWRSSSLLEEKMPRWAAEHRLEEKKTWLTNGFDVFECPPPKTENELLQMFYRQQEEIRRLRELLTQREVQAKQLELEIKNLRMGSEQL

(19) United States
(12) Patent Application Publication
(10) Pub. No.: US 2005/0059618 A1
(43) Pub. Date: Mar. 17, 2005

(54) Men protein, gst2, rab-rp1, csp, f-box protein lilina/fbl7, abc50, coronin, sec61 alpha, or vhappa1-1, or homologous proteins involved in the regulation of energy homeostasis

(76) Inventors: Karsten Eulenberg, Bovenden (DE); Arnd Steuernagel, Gottingen (DE); Thomas Hader, Gottingen (DE); Gunter Bronner, Gottingen (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/494,921

(22) PCT Filed: Nov. 8, 2002
(86) PCT No.: PCT/EP02/12518

(30) Foreign Application Priority Data
Nov. 8, 2001 (EP) 01126681.4
Nov. 9, 2001 (EP) 01126804.2
Nov. 13, 2001 (EP) 01126967.7
Nov. 20, 2001 (EP) 01127669.8
Nov. 23, 2001 (EP) 01127960.1
Nov. 23, 2001 (EP) 01127959.3
Nov. 28, 2001 (EP) 01128254.8
Dec. 13, 2001 (EP) 01129727.2
Dec. 19, 2001 (EP) 01130310.4
Jan. 14, 2002 (EP) 02000819.9

(51) Int. Cl. A61K 48/00; C12N 15/85

(52) U.S. Cl. 514/44; 435/455

(57) ABSTRACT

The present invention discloses Men protein, GST2, Rab-RP1, Csp, F-box protein Lilina/FBL7, ABC50, coronin, Sec61 alpha, or VhaPPA1-1 and homologous proteins regulating the energy homeostasis and the metabolism of triglycerides, and polynucleotides, which identify and encode the proteins disclosed in this invention. The invention also relates to the use of these sequences in the diagnosis, study, prevention, and treatment of diseases and disorders, for example, but not limited to, metabolic diseases such as obesity as well as related disorders such as eating disorder, cachexia, diabetes mellitus, hypertension, coronary heart disease, hypercholesterolemia, dyslipidemia, osteoarthritis, gallstones, cancers of the reproductive organs, and sleep apnea.

This invention relates to the use of nucleic acid sequences encoding malic enzyme (referred to as Men protein), Glutathione S-transferase 2 (referred to as GST2), Rab-related protein 1 (referred to as Rab-RP1), Cysteine string protein (referred to as Csp), CG11033 (referred to as F-box protein Lilina/FBL7), CG1703 (ABCF1, TSAP; referred to as ABC50), coro (referred to as coronin), Sec61 alpha, and VhaPPA1-1, or to mammalian, particularly human Men protein, GST2, Rab-RP1, Csp, F-box protein Lilina/FBL7, ABC50, coronin, Sec61 alpha, or VhaPPA1-1 homologous proteins (for example, NADP-dependent cytosolic malic enzyme 1 (ME1), NADP-dependent mitochondrial malic enzyme 3 (ME3), NAD(+)-dependent mitochondrial malic enzyme 2 (ME2), hematopoietic prostaglandin D2 synthase (PGDS), RAB32, RAB38, RAB7, cysteine string protein 2, gamma cysteine string protein, Beta cysteine string protein, F-box and leucine-rich repeat protein 11 (FBL11), JEMMA protein, PHD finger protein 2, protein with GenBank Accession Number AAC83407, ABC50 (TNF-alpha stimulated ABC protein), coronin 1B, coronin 1C, clipinE/coronin 6 type B, coronin 2A, coronin 2B, Sec61 alpha form 2, Sec61 alpha form 1, and vacuolar ATP synthase 21 kDa proteolipid subunit), and the polypeptides encoded thereby and effectors thereof and to the use thereof in the diagnosis, study, prevention, and treatment of diseases and disorders related to body-weight regulation, for example, but not limited to, metabolic diseases such as obesity as well as related disorders such as eating disorder, cachexia, diabetes mellitus, hypertension, coronary heart disease, hypercholesterolemia, dyslipidemia, osteoarthritis, gallstones, cancer, e.g. cancers of the reproductive organs, and sleep apnea.

There are several metabolic diseases of human and animal metabolism, eg., obesity and severe weight loss, that relate to energy imbalance where caloric intake versus energy expenditure is imbalanced. Obesity is one of the most prevalent metabolic disorders in the world. It is still a poorly understood human disease that becomes as a major health problem more and more relevant for western society. Obesity is defined as a body weight more than 20% in excess of the ideal body weight, frequently resulting in a significant impairment of health. It is associated with an increased risk for cardiovascular disease, hypertension, diabetes, hyperlipidaemia and an increased mortality rate. Besides severe risks of illness, individuals suffering from obesity are often isolated socially.

Obesity is influenced by genetic, metabolic, biochemical, psychological, and behavioral factors. As such, it is a complex disorder that must be addressed on several fronts to achieve lasting positive clinical outcome. Obese individuals are prone to ailments including: diabetes mellitus, hypertension, coronary heart disease, hypercholesterolemia, dyslipidemia, osteoarthritis, gallstones, cancers of the reproductive organs, and sleep apnea.

Since obesity is not to be considered as a single disorder but a heterogeneous group of conditions with (potential) multiple causes, it is also characterized by elevated fasting plasma insulin and an exaggerated insulin response to oral glucose intake (Koltermann, J. Clin. Invest 65, 1980, 1272-1284) and a clear involvement of obesity in type 2 diabetes mellitus can be confirmed (Kopelman, Nature 404, 2000, 635-643).

The molecular factors regulating food intake and body weight balance are incompletely understood. Even if several candidate genes have been described which are supposed to influence the homeostatic system(s) that regulate body mass/weight, like leptin, VCPI, VCPL, or the peroxisome proliferator-activated receptor (PPAR)-gamma co-activator, the distinct molecular mechanisms and/or molecules influencing obesity or body weight/body mass regulations are not known. In addition, several single-gene mutations resulting in obesity have been described in mice, implicating genetic factors in the etiology of obesity. (Friedman and Leibel, 1990, Cell 69: 217-220). In the obese mouse model, a single gene mutation (obese) results in profound obesity, which is accompanied by diabetes (Friedman et. al., 1991, Genomics 11: 1054-1062).

Therefore, the technical problem underlying the present invention was to provide for means and methods for modulating (pathological) metabolic conditions influencing body-weight regulation and/or energy homeostatic circuits. The solution to said technical problem is achieved by providing the embodiments characterized in the claims.

Accordingly, the present invention relates to genes with novel functions in body-weight regulation, energy homeostasis, metabolism, and obesity. The present invention discloses specific genes involved in the regulation of body-weight, energy homeostasis, metabolism, and obesity, and thus in disorders related thereto such as eating disorder, cachexia, diabetes mellitus, hypertension, coronary heart disease, hypercholesterolemia, dyslipidemia, osteoarthritis, gallstones, cancer, e.g. cancers of the reproductive organs, and sleep apnea. The present invention describes the human Men protein, GST2, Rab-RP1, Csp, F-box protein Lilina/FBL7, ABC50, coronin, Sec61 alpha, or VhaPPA1-1 (herein refered to as 'proteins of the invention) homologous genes and proteins encoded thereby as being involved in those conditions mentioned above.

The term “GenBank Accession number” relates to National Center for Biotechnology Information (NCBI) GenBank database entries (Benson et al, Nucleic Acids Res. 28, 2000, 15-18).

Men

The Drosophila Men gene with GadFly Accession Number CG10120 encodes for a malate dehydrogenase (oxaloacetate decarboxylating) (NADP+) (EC:1.1.1.40). Men is highly conserved and might be differentially spliced in addition to other malic enzymes like Mdh (GenBank Accession Number AE003759). We found that Drosophila Men protein is most homologous to human and mouse Men proteins. Men is the structural gene for malic enzyme, which is identical to (S)-Malate: NADP+ oxidoreductase (MEN). The enzyme is known to provide NADPH for lipogenesis; NADPH levels in fly larvae are increased by dietary carbohydrate and decreased by dietary lipid. Highest specific activity found in larval fat body and, among cellular fractions, in the cytosol (Geer et al. B. W. (1979) Biochem Genet 17(9-10):867-879).

The human Men gene encodes for the cytosolic form of an enzyme of the citrate cycle (Malate+NAD⁺→Oxalacetate+NADH+H⁺) that is localised in mitochondria and is NAD+ coupled. In addition, there is also a mitochbndrial form of malate dehydrogenase that is NADH coupled. Alternatively, it might encode for the NADP⁺ dependent malic enzyme that catalyzes Malate+NADP⁺→Pyruvate+CO₂+NADPH.

GST2

GSH-dependent prostaglandin D(2) synthase (GST2) enzymes represent the only vertebrate members of class Sigma glutathione S-transferases (GSTs) identified to date (see, Kanaoka et al., 2000, Eur. J. Biochem. 267:3315-3322). Orthologous human and rat GSH-dependent GST2 were both shown to catalyse specifically the isomerization of prostaglandin (PG) H(2), a common precursor of various prostanoids, to produce PGD2 as a major prostanoid in a variety of tissues (review, see, for example, Urade & Hayaishi Vitam Horm 2000;58:89-120). Each transferase also exhibited GSH-conjugating and GSH-peroxidase activities (Jowsey et al., Biochem J 2001 359(Pt 3):507-16).

PGD2 has various functions in the peripheral tissues, such as prevention of platelet aggregation and induction of vasodilatation and bronchoconstriction. PGD2 is released from mast cells stimulated by various immunological stimulants and functions as a lipid mediator in allergy and inflammation. PGD2 is further converted to 9 alpha, 11 beta-PGF2 or the J series of prostanoids. The J series of PGs were found to have an antiproliferative effect against tumor cells (see, for example, Fukushima et al, 1994, Ann. N.Y. Acad. Sci 744:161-165]. A PGJ2 metabolite, 15-deoxy-D12,14-PGJ2, which promotes adipocyte differentiation, was identified as a natural ligand for the peroxisome proliferator-activated receptor (PPAR) gamma (see, for example, Kliewer et al. 1995, Cell 83:813-819). The ligand activation of PPAR gamma was found to regulate macrophage and monocyte functions (see, for example, Huang et al., 1999, Nature 400:378-382).

Two types of PGD synthase exist, the lipocalin-type enzyme and the hematopoietic enzyme. Hematopoietic PGD synthase is widely distributed in the peripheral tissues and localized in the antigen-presenting cells, mast cells, and megakaryocytes. The hematopoietic enzyme is the first recognized vertebrate homolog of the sigma class of glutathione S-transferase (see, Kanaoka et al., Eur J Biochem 2000 267(11):3315-22). X-ray crystallographic analyses and generation of gene-knockout and transgenic mice for each enzyme have been performed.

Hepatic glutathione S-transferase activity was studied in obese mice (Wolff & Suber, Proc Soc Exp Biol Med 1986 181(4):535-41). It was found that the hepatic glutathione S-transferase activity of yellow Avy/a (YS X VY) F-1 hybrid female mice was decreased compared to the activity measured black a/a female mice which was associated with the obesity of the yellow mice.

RabRP1

Rab proteins constitute a family of GTP-binding proteins that are located in distinct intracellular compartments and play a role in the regulation of vesicular trafficking, including exocytosis and endocytosis (see, for review, Armstrong, Int J Biochem Cell Biol 2000 32(3):303-7 J). More than 50 mammalian Rab proteins are known, many with transport step-specific localisation. Through their effectors, Rab GTPases regulate vesicle formation, actin- and tubulin-dependent vesicle movement, and membrane fusion. A number of Rab GTPases are conserved from yeast to humans. Yeast mutations in Rab gene homologs cause defects in vesicular transport similar to those observed in beige (bg) mice, the murine Hermansky-Pudlak syndrome model.

Rab-related small GTP-binding protein (Rab38) has been localized to the lung, especially alveolar type II cells and bronchial epithelial cells, suggesting a role in vesicular transport in terminal airway epithelium (see Osanai et al. Am J Pathol 2001 158(5):1665-75). In addition, Rab38 is showing a predominant mRNA expression in melanocytes, a cell-specific expression pattern likely related to melanosomal transport and docking (see Jager et al. Cancer Res 2000;60(13):3584-91). Among the family of rab proteins, rab38 has a unique COOH terminus which would allow posttranslational farnesylation and palmitoylation, lipid modifications normally occurring in ras proteins but not in other rab proteins (see Jager et al. 2000, supra).

A review by Cormont and Le Marchand-Brustel discusses the role of small G-proteins in the regulation of glucose transport (see, Mol Membr Biol 2001 July-September;18(3):213-20A). They discuss that insulin increases the rate of glucose transport into fat and muscle cells by stimulating the translocation of intracellular Glut 4-containing vesicles to the plasma membrane, along with an increase in the amount of the facilitative glucose transporter Glut 4 at the cell surface, allowing for an enhanced glucose uptake. This process requires a continuous cycling through the early endosomes, a Glut 4 specific storage compartment and the plasma membrane. The main effect of insulin is to increase the rate of Glut 4 trafficking from its specific storage compartment to the plasma membrane. The whole phenomenon involves signal transduction from the insulin receptor, vesicle trafficking (sorting and fusion processes) and actin cytoskeleton modifications, which are all supposed to require small GTPases.

A member of the Rab 3 subfamily of small GTP-binding proteins, Rab 3D, in rat adipose cells, has been postulated to be involved in insulin-stimulated GLUT4 exocytosis (Guerre-Millo et al. Biochem J. 1997;321 (Pt 1):89-93). Rab 3D is overexpressed in adipose cells of obese (fa/fa) Zucker rats, in a tissue- and isoform-specific manner. The pathophysiological significance of this defect remains elusive which could form the molecular basis for altered adipose secretory function in obesity.

CSP

Cysteine-string protein (Csp) is a major synaptic vesicle and secretory granule protein first discovered in Drosophila and Torpedo (for review, see, for example, Chamberlain & Burgoyne, 2000, J Neurochem 2000 74(5):1781-9 RD), and were subsequently identified from Xenopus, Caenorhabditis elegans, and mammalian species. Studies from the null mutant in Drosophila have shown that Csp is required for viability of the organism. It has been also shown that Csp plays a key role in neurotransmitter release. Amorphic Drosophila mutations have been isolated which affect the larval neuromuscular junction and are conditional temperature sensitive paralytic, conditional temperature sensitive neurophysiology defective and recessive semi-lethal. Furthermore, other studies have directly implicated Csp in regulated exocytosis in mammalian neuroendocrine and endocrine cell types, and its distribution suggests a general role in regulated exocytosis. Csps possess a cysteine-string domain that is highly palmitoylated and confers membrane targeting. In addition, Csps have a conserved “J” domain that mediates binding to an activation of the Hsp70/Hsc70 chaperone ATPases. Targets for Csp include the vesicle protein VAMP/synaptobrevin and the plasma membrane protein syntaxin 1.

It has been shown that the cysteine-string protein is associated with the plasma membrane in 3T3-L1 adipocytes but not with intracellular Glut4-storage vesicles. Csp1 interacts with the t-SNARE protein syntaxin 4 which is an important mediator of insulin-stimulated fusion with the plasma membrane, suggesting that Csp1 may play a regulatory role in this process. In contrast, syntaxin 1A binds to both Csp isoforms (Csp1 and Csp2), with higher affinity for the Csp2 protein (see, Chamberlain et al., 2001, J Cell Sci;114(Pt 2):445-55).

F-Box

The Drosophila gene CG11033 encodes for a F-box-like protein involved in neuropeptide signaling that is required for normal circadian locomotor rhythms in Drosophila. Interpro analysis of this gene reveals cytochrome c family heme-binding site, an F-box protein Lilina/FBL7 domain, CXXC zinc finger and a glycin-rich region domains. We found that F-box protein Lilina/FBL7 is most homologous to human F-box protein Lilina/FBL7 protein (GenBank Accession Number NP_{—}036440.1) which was recently cloned by Ilyin et al., 2000 (Genomics 67(1):40-47). F-box proteins are components of the SCF ubiquitin-protein ligase complex which functions in several biological processes like cell cycle control, apoptosis, transcription, and signal transduction. It has been shown that the SCF ubiquitin-protein ligase complex is essential for the NF-kappaB, Wnt/Wingless, and Hedgehog signaling pathways (Maniatis T., (1999) Genes Dev. 13(5):505-510), signaling pathways that are involved in metabolism.

ABC50

The ABC50 is a member of the ATP-binding cassette (ABC) proteins. Unlike the majority of ABC proteins, which are membrane-associated transporters, ABC50 associates with the ribosome in an ATP-dependent manner (see, Tyzack et al., J. Biol. Chem. 275: 137-45). ABC 50 has been shown to interact with eukaryotic initiation factor 2 (elF2), which plays a key role in the process of translation initiation and in its control. ABC50 is related to GCN20 and eEF3, two yeast ABC proteins that are not membrane-associated transporters and are instead implicated in mRNA translation and/or its control. Therefore, ABC50 is considered as an ABC protein with a likely function in mRNA translation, which associates with elF2 and with ribosomes. A role of ABC50 in the enhancement of protein synthesis has been postulated that follows TNF-alpha treatment of synoviocytes and thus participates in the inflammatory processes mediated by this cytokine (Richard et al. Genomics, 1998, 53:137-45).

Coronin

Coronin belongs to a family of actin-associated proteins and was first isolated from Dictyostelium, but similar proteins have been identified in many species and individual cell types (for review, see de Hostos, Trends Cell Biol 1999 9(9):345-350). Coronin is an actin-binding protein, which contains WD (Trp-Asp) repeats and a coiled-coil motif, and plays a role in regulating organization of the actin cytoskeletal network. Coronin localizes to the cell periphery, is involved in lamellipodium extension, and has an implicated role in cytokinesis, cell motility and phagocytosis. During phagocytosis coronin is recruited together with PI 3-kinase to membranes of nascent and early phagosomes co-localizing with the actin cytoskeleton, confirming that coronin contributes to phagocytosis (see, for example, Didichenko et al., FEBS Lett. 2000 24;485(2-3):147-152). Although the existence of coronin in higher eukaryotes has been reported, its function in vertebrate cells has not been elucidated.

Sec61 Alpha

The Sec61 complex is a central component of the endoplasmic reticulum (ER) translocation site (translocon). The complex consists of three subunits: Sec61 alpha, Sec61 beta and Sec61 gamma, at least two of which (alpha and beta) are adjacent to nascent proteins during membrane insertion. Sec61 alpha functions as the major component of a transmembrane channel formed by oligomers of the Sec61 complex. This channel is the site of secretory protein translocation and membrane protein integration at the ER membrane. Sec61 alpha is a polytopic integral membrane protein (see, for example, Knight and High, (1998) Biochem J 331 (Pt 1):161-167). Sec61 alpha has SecY protein domains. Sec61 alpha was reported to interact with Grp170, Grp94, BiP/Grp78, calreticulin, and protein disulfide isomerase (see, Dierks et al., (1996) EMBO J 15(24):6931-6942).

Mitchell et al. describe that Apoprotein B100 has a prolonged interaction with the translocon during which its lipidation and translocation change from dependence on the microsomal triglyceride transfer protein to independence (Proc Natl Acad Sci U S A. 1998 95(25):14733-8). Pariyarath et al. discuss the co-translational interactions of apoprotein B with the ribosome and translocon during lipoprotein assembly or targeting to the proteasome (see, J Biol Chem. 2001 Jan. 5;276(1):541-50).

VhaPPA1-1

The Drosophila VhaPPa1-1 encodes for a hydrogen-transporting two-sector ATPase which is a component of the hydrogen-transporting ATPase Vo domain. Intrapro analysis reveals vacuolar ATP synthase 16 kD subunit and ATP synthase subunit C protein domains. VhaPPa1-1 is most homologous to mouse vacuolar proton-translocating ATPase 21 kDa subunit and to human ATPase, H⁺ transporting, lysosomal 21 KD subunit. Vacuolar ATPases are involved in the lysosomal transport and metabolism of lipoproteins like LDL (see, for example, U.S. Pat. No. 6,107,462). The proteolipid domain of vacuolar H(+)-ATPase (V-ATPase) plays a major role in H⁺ transport in microvesicles and other acidic organelles. Nishigori et al Genomics 1998 Jun. 1;50(2):222-8 have cloned the second human proteolipid of the V-ATPase (designated hATP6F), a homologue of the Saccharomyces cerevisiae proteolipid VMA16, which is an essential subunit of yeast V-ATPase. hATP6F is a hydrophobic protein with five putative transmembrane segments, having 61% amino acid identity and 83% similarity to the yeast protein, except in the N-terminus, and contains a conserved glutamic acid residue (Glu98) that is essential for H⁺-transporting activity. The epitope-tagged 23-kDa protoelipid was localized in endomembrane organelles in CHO cells, as expected for a component of a vacuolar-type proton pump (Sun-Wada et al. Gene 2001 274(1-2):93-99).

So far, it has not been described that malic enzyme (referred to as Men protein), Glutathione S-transferase 2 (referred to as GST2), Rab-related protein 1 (referred to as Rab-RP1), Cysteine string protein (referred to as Csp), CG11033 (referred to as F-box protein Lilina/FBL7), CG1703 (ABCF1, TSAP; referred to as ABC50), coro (referred to as coronin), Sec61 alpha, and VhaPPA1-1, or human Men protein, GST2, Rab-RP1, Csp, F-box protein Lilina/FBL7, ABC50, coronin, Sec61 alpha, or VhaPPA1-1 homologous proteins are involved in the regulation of energy homeostasis and body-weight regulation and related disorders, and thus, no functions in metabolic diseases and other diseases as listed above have been discussed. In this invention we demonstrate that the correct gene dose of Men protein, GST2, Rab-RP1, Csp, F-box protein Lilina/FBL7, ABC50, coronin, Sec61 alpha, or VhaPPA1-1 is essential for maintenance of energy homeostasis. A genetic screen was used to identify that mutation of Men protein, GST2, Rab-RP1, Csp, F-box protein Lilina/FBL7, ABC50, coronin, Sec61 alpha, or VhaPPA1-1 homologous genes cause obesity, reflected by a significant increase of triglyceride content, the major energy storage substance.

Polynucleotides encoding a protein with homologies to Men protein, GST2, Rab-RP1, Csp, F-box protein Lilina/FBL7, ABC50, coronin, Sec61 alpha, or VhaPPA1-1 are suitable to investigate diseases and disorders as described above. Molecules related to Men protein, GST2, Rab-RP1, Csp, F-box protein Lilina/FBL7, ABC50, coronin, Sec61 alpha, or VhaPPA1-1 are suitable for providing new compositions useful in diagnosis, treatment, and prognosis of diseases and disorders as described above.

Before the present proteins, nucleotide sequences, and methods are described, it is understood that this invention is not limited to the particular methodology, protocols, cell lines, vectors, and reagents described as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies which are reported in the publications which might be used in connection with the invention. Nothing herein is to be construed as an admission that the invention is not entitled to antedate such disclosure.

In this invention we particularly refer to Malic enzyme (referred to as Men protein), Glutathione S-transferase 2 (referred to as GST2), Rab-related protein 1 (referred to as Rab-RP1), Cysteine string protein (referred to as Csp), CG11033 (referred to as F-box protein Lilina/FBL7), CG1703 (ABCF1, TSAP; referred to as ABC50), coro (referred to as coronin), Sec61 alpha, and VhaPPA1-1, and Men protein, GST2, Rab-RP1, Csp, F-box protein Lilina/FBL7, ABC50, coronin, Sec61 alpha, and VhaPPA1-1 homologous proteins (for example, NADP-dependent cytosolic malic enzyme 1 (ME1), NADP-dependent mitochondrial malic enzyme 3 (ME3), NAD(+)-dependent mitochondrial malic enzyme 2 (ME2), hematopoietic prostaglandin D2 synthase (PGDS), RAB32, RAB38, RAB7, cysteine string protein 2, gamma cysteine string protein, Beta cysteine string protein, F-box and leucine-rich repeat protein 11 (FBL11), JEMMA protein, PHD finger protein 2, protein with GenBank Accession Number AAC83407, ABC50 (TNF-alpha stimulated ABC protein), coronin 1B, coronin 1C, clipinE/coronin 6 type B, coronin 2A, coronin 2B, Sec61 alpha form 2, Sec61 alpha form 1, and vacuolar ATP synthase 21 kDa proteolipid subunit), which include Drosophila and mammalian, preferably human, homologous polypeptides or proteins or sequences encoding those proteins.

Especially Preferred Embodiments are:

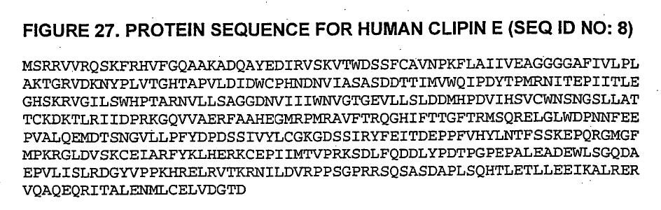

Drosophila Men (GadFly Accession Number CG10120), human NADP-dependent cytosolic malic enzyme 1 (ME1; GenBank Accession No. NM_{—}002395 for the cDNA, NP_{—}002386 for the protein), or NADP-dependent mitochondrial malic enzyme 3 (ME3; GenBank Accession No. NM_{—}006680 for the cDNA, NP_{—}006671 for the protein), or NAD(+)-dependent mitochondrial malic enzyme 2 (ME2; GenBank Accession No. NM_{—}002396.2 for the cDNA, NP_{—}002387 for the protein),
Drosophila Gst2 (GadFly Accession Number CG8938), human hematopoietic prostaglandin D2 synthase (PGDS; GenBank Accession No. NM_{—}014485 for the cDNA, NP_{—}055300 for the protein), mouse hematopoietic prostaglandin D2 synthase 2 (Ptgds2; GenBank Accession No. NM_{—}019455 for the cDNA; glutathione-requiring prostaglandin D synthase), rat hematopoietic prostaglandin D2 synthase 2 (Ptgds2; GenBank Accession No. NM_{—}031644 for the cDNA; glutathione-requiring prostaglandin D synthase),
Drosophila RabRP1 (GadFly Accession Number CG8024), human Rab32 (GenBank Accession No. NM_{—}006834 for the cDNA, NP_{—}006825 for the protein, formerly XM_{—}004076, human Rab38 (GenBank Accession No. NM_{—}022337 for the cDNA, NP_{—}071732 for the protein, formerly XM_{—}015771, mouse Rab32 (GenBank Accession No. NM_{—}026405 for the cDNA), mouse Rab38 (GenBank Accession No. NM_{—}028238 for the cDNA), human Rab7 (GenBank Accession No. NM_{—}003929 for the cDNA, NP_{—}003920 for the protein),
Drosophila Csp (GadFly Accession Number CG6395), human Csp (EnsEMBL accession number ENST00000217123 for the cDNA; GenBank Accession Number CAC15495.1 for the protein), human cysteine string protein 1 (GenBank Accession No. S70515 for the protein), human gamma cysteine string protein (unnamed protein product; GenBank Accession No. AK097736 for the cDNA, BAC05155 for the protein), human Beta cysteine string protein (GenBank Accession No. Q9UF47),
Drosophila F-box protein (GadFly Accession Number CG11033), human F-box and leucine-rich repeat protein 11 (GenBank Accession No. NM_{—}012308 for the cDNA, NP_{—}036440.1 for the protein) human JEMMA protein (GenBank Accession No. CAD30700 for the protein), PDH finger protein 2 (GenBank Accession Number NM_{—}005392 for the cDNA, NP_{—}005383 for the protein), human protein similar to several hypothetical proteins (GenBank Accession No. AAC83407 for the protein),
Drosophila ABC50 (GadFly Accession Number CG1703), human TNF-alpha stimulated ABC protein (GenBank Accession No. AF027302 for the cDNA, AAC70891 for the protein), rat ABC50 (GenBank Accession No. AF293383 for the cDNA),
Drosophila coro (GadFly Accession Number CG9446), human actin-binding protein coronin 1B (GenBank Accession No. NM_{—}020441 for the cDNA, NP_{—}065174 for the protein, formerly GenBank Accession No. BC006449), human actin-binding protein coronin 1C (GenBank Accession No. NM_{—}014325 for the cDNA, NP_{—}055140 for the protein; GenBank Accession No. BC002342), human coronin homologue (GenBank Accession No. X89109 for the cDNA, CAA61482 for the protein), human clipinE/coronin 6 type B (see FIG. 27; Seq ID NO: 8), human Coronin 2A (GenBank Accession No. Q92828 for the protein), human Coronin 2B (GenBank Accession No. Q9UQ03 for the protein),
Drosophila sec61 alpha (GadFly Accession Number CG9539), human Sec61 alpha form 2 protein (GenBank Accession No. NM_{—}018144 for the cDNA, NP_{—}060614 for the protein, formerly GenBank Accession No. AF346603), human Sec61 alpha form 1 protein (GenBank Accession No. NEU NM_{—}013336.2 for the cDNA, NP_{—}037468 for the protein, formerly AF346602), mouse Sec61 alpha-2 protein (GenBank Accession No. AF222748), mouse Sec61 isoform 1 protein (GenBank Accession No. AF145253),
Drosophila VhaPPA1-1 (GadFly Accession Number CG7007), human ATPase, H+transporting, lysosomal 21 kD (vacuolar protein pump) protein (GenBank Accession No. NM_{—}004047 for the cDNA, NP_{—}004038 for the protein), and mouse ATPase, H+transporting, lysosomal 21 kD (vacuolar protein pump) protein (GenBank Accession No. NM 033617 for the cDNA) as proteins of the invention.

The present invention discloses proteins, which are regulating the energy homeostasis and fat metabolism especially the metabolism and storage of triglycerides, and polynucleotides, which identify and encode the proteins disclosed in this invention. The invention also relates to vectors, host cells, antibodies, and recombinant methods for producing the polypeptides and polynucleotides of the invention. The invention also relates to the use of these sequences and effector molecules thereof in the diagnosis, study, prevention, and treatment of diseases and disorders, for example, but not limited to, metabolic diseases such as obesity as well as related disorders such as eating disorder, cachexia, diabetes mellitus, hypertension, coronary heart disease, hypercholesterolemia, dyslipidemia, osteoarthritis, gallstones, cancer, e.g. cancers of the reproductive organs, and sleep apnea.

The proteins of the invention and nucleic acid molecules coding therefor are obtainable from insect or vertebrate species, e.g. mammals or birds. Particularly preferred are homologous nucleic acids, particularly nucleic acids encoding a human homologous protein of the invention as described above.

The invention particularly relates to a nucleic acid molecule encoding a polypeptide contributing to regulating the energy homeostasis and the metabolism of triglycerides, wherein said nucleic acid molecule comprises
(a) the nucleotide sequence encoding a protein of the invention and/or a sequence complementary thereto,
(b) a nucleotide sequence which hybridizes at 50° C. in a solution containing 1×SSC and 0.1% SDS to a sequence of (a),
(c) a sequence corresponding to the sequences of (a) or (b) within the degeneration of the genetic code,
(d) a sequence which encodes a polypeptide which is at least 85%, preferably at least 90%, more preferably at least 95%, more preferably at least 98% and up to 99.6% identical to the amino acid sequence of a protein of the invention,
(e) a sequence which differs from the nucleic acid molecule of (a) to (d) by mutation and wherein said mutation causes an alteration, deletion, duplication and/or premature stop in the encoded polypeptide or
(f) a partial sequence of any of the nucleotide sequences of (a) to (e) having a length of at least 15 bases, preferably at least 20 bases, more preferably at least 25 bases and most preferably at least 50 bases.

The invention is based on the finding that the proteins of the invention and the polynucleotides encoding these, are involved in the regulation of triglyceride storage and therefore energy homeostasis. The invention describes the use of compositions comprising these polynucleotides, polypeptides or effectors thereof, e.g. antibodies, biologically active nucleic acids, such as antisense molecules, RNAi molecules or ribozymes, aptamers, peptides or low-molecular weight molecules or other receptors of the polypeptides or polynucleotides for the diagnosis, study, prevention, or treatment of diseases and disorders related thereto, including metabolic diseases such as obesity as well as related disorders such as eating disorder, cachexia, diabetes mellitus, hypertension, coronary heart disease, hypercholesterolemia, dyslipidemia, osteoarthritis, gallstones, cancer, e.g. cancers of the reproductive organs, and sleep apnea.

Accordingly, the present invention relates to genes with novel functions in body-weight regulation, energy homeostasis, metabolism, and obesity. To find genes with novel functions in energy homeostasis, metabolism, and obesity, a functional genetic screen was performed with the model organism Drosophila melanogaster (Meigen). Drosophila melanogaster is one of the most intensively studied organisms in biology and serves as a model system for the investigation of many developmental and cellular processes common to higher eukaryotes, including humans (see, for example, Adams et al., Science 287: 2185-2195 (2000)). The success of Drosophila melanogaster as a model organism is largely due to the power of forward genetic screens to identify the genes that are involved in a biological process (see, Johnston Nat Rev Genet 3: 176-188 (2002); Rorth, Proc Natl Acad Sci U S A 93: 12418-12422 (1996)). One resource for screening was a proprietary Drosophila melanogaster stock collection of EP-lines. The P-vector of this collection has Gal4-UAS-binding sites fused to a basal promoter that can transcribe adjacent genomic Drosophila sequences upon binding of Gal4 to UAS-sites. This enables the EP-line collection for overexpression of endogenous flanking gene sequences. In addition, without activation of the UAS-sites, integration of the EP-element into the gene is likely to cause a reduction of gene activity, and allows determining its function by evaluating the loss-of-function phenotype.

Triglycerides are the most efficient storage for energy in cells, and are significantly increased in obese patients. In this invention, we have used a genetic screen to identify, that mutations of a gene encoding a protein of the invention or homologous genes cause changes in the body weight which is reflected by a significant change in the triglyceride levels. In order to isolate genes with a function in energy homeostasis, several thousand proprietary and publicly available EP-lines were tested for their triglyceride content after a prolonged feeding period (illustrated in more detail in the EXAMPLES). Lines with significantly changed triglyceride content were selected as positive candidates for further analysis.

In this invention, the content of triglycerides of a pool of flies with the same genotype after feeding for six days was analyzed using a triglyceride assay, as, for example, but not for limiting the scope of the invention, is described in more detail below in the examples section. The change of triglyceride content due to the loss of a gene function suggests gene activities in energy homeostasis in a dose dependent manner that controls the amount of energy stored as triglycerides.

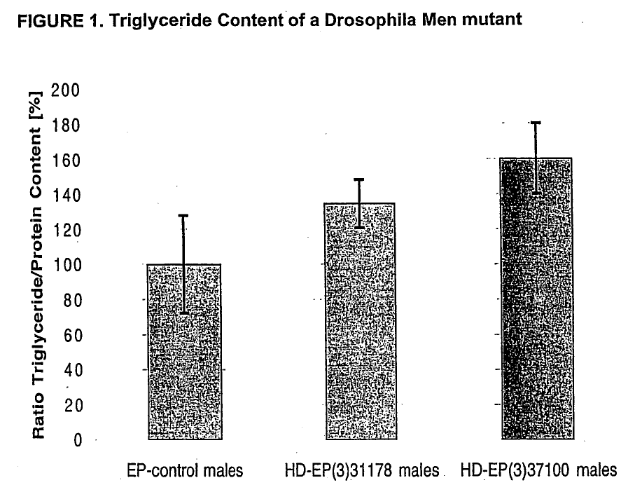

The results of the triglyceride content analysis are shown in FIGS. 1, 5, 8, 12, 17, 22, 25, 31, and 33. We found that homozygous HD-EP(3)31178, HD-EP(3)37100, EP(2)0641, HD-EP(2)26782, EP(3)3141, HD-EP(3)31735, HD-EP(X)10216, HD-EP(2)26155, EP(2)2108, EP(2)2567, and EP(3)3504 flies have a higher triglyceride content than the controls (average triglyceride levels). Therefore, the very likely loss of a gene activity in the gene loci, where the EP-vectors are integrated, is responsible for changes in the metabolism of the energy storage triglycerides, therefore representing in all cases an obese fly model. The increase of triglyceride content due to the loss of a gene function suggests gene activities in energy homeostasis in a dose dependent manner that controls the amount of energy stored as triglycerides.

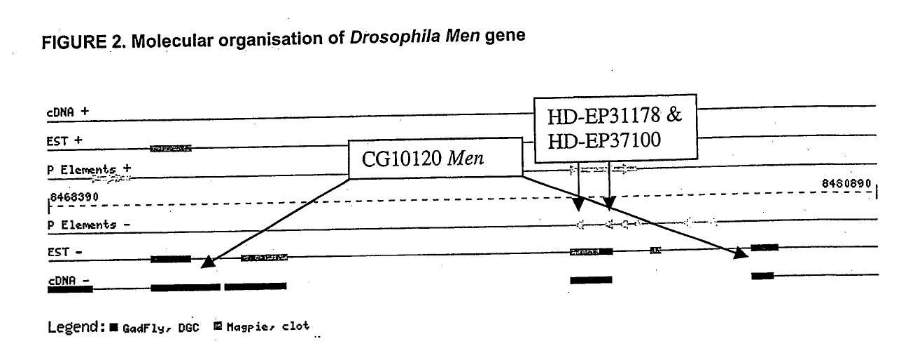

Nucleic acids encoding the proteins of the present invention were identified using a plasmid-rescue technique. Genomic DNA sequences were isolated that are localised directly 3′ to the EP vectors (herein HD-EP(3)31178, HD-EP(3)37100, EP(2)0641, HD-EP(2)26782, EP(3)3141, HD-EP(3)31735, HD-EP(X)10216, HD-EP(2)26155, EP(2)2108, EP(2)2567, or EP(3)3504) integration. Using those isolated genomic sequences public databases like Berkeley Drosophila Genome Project (GadFly, see also FlyBase (1999) Nucleic Acids Research 27:85-88) were screened thereby confirming the homozygous viable integration side of the vectors into the transcription units of the genes FIGS. 2, 6, 9, 13, 18, 23, 26, 32, and 34 show the molecular organisation of these gene loci.

The present invention is further describing polypeptides comprising the amino acid sequences of the proteins of the invention. Based upon homology, the proteins of the invention and each homologous protein or peptide may share at least some activity. No functional data described the regulation of body weight control and related metabolic diseases such as obesity are available in the prior art for the genes of the invention.

The proteins of the invention and homologous proteins and nucleic acid molecules coding therefor are obtainable from insect or vertebrate species, e.g. mammals or birds. Particularly preferred are nucleic acids encoding the human homologs of the proteins of the invention. The present invention is describing polypeptides comprising the amino acid sequences of the proteins of the invention. Comparisons (ClustalX 1.8 analysis or ClustalW 1.82 analysis, see for example Thompson J. D. et al., (1994) Nucleic Acids Res. 22(22):4673-4680; Thompson J. D., (1997) Nucleic Acids Res 25(24):4876-4882; Higgins, D. G. et al., (1996) Methods Enzymol. 266:383-402) between the respective proteins of different species (human and Drosophila) were conducted. Based upon homology, the Drosophila proteins of the invention and each homologous protein or peptide may share at least some activity. No functional data described the regulation of body weight control and related metabolic diseases such as obesity are available in the prior art for the genes claimed in this invention.

Further, we show that the mouse homologues of the genes encoding the proteins of the invention are regulated by fasting, by high fat diet, or by genetically induced obesity. Furthermore, the expression of the mouse homologues of Men, Rab32, Csp, ABC50, and vATPase is upregulated during adipocyte differentiation in vitro, and the expression of the mouse homologue of F-box is downregulated during adipocyte differentiation in vitro (see EXAMPLES).

The invention also encompasses polynucleotides that encode the proteins of the invention and homologous proteins. Accordingly, any nucleic acid sequence, which encodes the amino acid sequences of the proteins of the invention, can be used to generate recombinant molecules that express the proteins of the invention. In a particular embodiment, the invention encompasses the polynucleotide comprising the nucleic acid sequence encoding the Drosophila or human proteins of the invention. It will be appreciated by those skilled in the art that as a result of the degeneracy of the genetic code, a multitude of nucleotide sequences encoding the proteins of the invention, some bearing minimal homology to the nucleotide sequences of any known and naturally occurring gene, may be produced. Thus, the invention contemplates each and every possible variation of nucleotide sequence that could be made by selecting combinations based on possible codon choices. These combinations are made in accordance with the standard triplet genetic code as applied to the nucleotide sequences of the naturally occurring proteins of the invention, and all such variations are to be considered as being specifically disclosed. Although nucleotide sequences which encode the proteins of the invention and their variants are preferably capable of hybridising to the nucleotide sequences of the naturally occurring proteins of the invention under appropriately selected conditions of stringency, it may be advantageous to produce nucleotide sequences encoding the proteins of the invention or their derivatives possessing a substantially different codon usage. Codons may be selected to increase the rate at which expression of the peptide occurs in a particular prokaryotic or eukaryotic host in accordance with the frequency with which particular codons are utilised by the host. Other reasons for substantially altering the nucleotide sequence encoding the proteins of the invention and their derivatives without altering the encoded amino acid sequences include the production of RNA transcripts having more desirable properties, such as a greater half-life, than transcripts produced from the naturally occurring sequences. The invention also encompasses production of DNA sequences, or portions thereof, which encode the proteins of the invention and their derivatives, entirely by synthetic chemistry. After production, the synthetic sequence may be inserted into any of the many available expression vectors and cell systems using reagents that are well known in the art at the time of the filing of this application. Moreover, synthetic chemistry may be used to introduce mutations into a sequence encoding the proteins of the invention any portion thereof.

Also encompassed by the invention are polynucleotide sequences that are capable of hybridising to the claimed nucleotide sequences, and in particular, those of the polynucleotide encoding the proteins of the invention under various conditions of stringency. Hybridisation conditions are based on the melting temperature (Tm) of the nucleic acid binding complex or probe, as taught in Wahl, G. M. and S. L. Berger (1987: Methods Enzymol. 152:399-407) and Kimmel, A. R. (1987; Methods Enzymol. 152:507-511), and may be used at a defined stringency. Preferably, hybridization under stringent conditions means that after washing for 1 h with 1×SSC and 0.1% SDS at 50° C., preferably at 55° C., more preferably at 62° C. and most preferably at 68° C., particularly for 1 h in 0.2×SSC and 0.1% SDS at 50° C., preferably at 55° C., more preferably at 62° C. and most preferably at 68° C., a positive hybridization signal is observed. Altered nucleic acid sequences encoding the proteins of the invention which are encompassed by the invention include deletions, insertions, or substitutions of different nucleotides resulting in a polynucleotide that encodes the same or functionally equivalent proteins of the invention.

The encoded proteins may also contain deletions, insertions, or substitutions of amino acid residues, which produce a silent change and result in functionally equivalent proteins of the invention. Deliberate amino acid substitutions may be made on the basis of similarity in polarity, charge, solubility, hydrophobicity, hydrophilicity, and/or the amphipathic nature of the residues as long as the biological activity of the proteins of the invention is retained. For example, negatively charged amino acids may include aspartic acid and glutamic acid; positively charged amino acids may include lysine and arginine; and amino acids with uncharged polar head groups having similar hydrophilicity values may include leucine, isoleucine, and valine; glycine and alanine; asparagine and glutamine; serine and threonine; phenylalanine and tyrosine.

Also included within the scope of the present invention are alleles of the genes encoding the proteins of the invention. As used herein, an allele or allelic sequence is an alternative form of the gene, which may result from at least one mutation in the nucleic acid sequence. Alleles may result in altered mRNAs or polypeptides whose structures or function may or may not be altered. Any given gene may have none, one, or many allelic forms. Common mutational changes, which give rise to alleles, are generally ascribed to natural deletions, additions, or substitutions of nucleotides. Each of these types of changes may occur alone, or in combination with the others, one or more times in a given sequence.

The nucleic acid sequences encoding the proteins of the invention may be extended utilising a partial nucleotide sequence and employing various methods known in the art to detect upstream sequences such as promoters and regulatory elements. For example, one method which may be employed, “restriction-site”. PCR, uses universal primers to retrieve unknown sequence adjacent to a known locus (Sarkar, G. (1993) PCR Methods Applic. 2:318-322). Inverse PCR may also be used to amplify or extend sequences using divergent primers based on a known region (Triglia, T. et al. (1988) Nucleic Acids Res. 16:8186). Another method which may be used is capture PCR which involves PCR amplification of DNA fragments adjacent to a known sequence in human and yeast artificial chromosome DNA (Lagerstrom, M. et al. (PCR Methods Applic. 1:111-119). Another method which may be used to retrieve unknown sequences is that of Parker, J. D. et al. (1991; Nucleic Acids Res. 19:3055-3060). Additionally, one may use PCR, nested primers, and PROMOTERFINDER libraries to walk in genomic DNA (Clontech, Palo Alto, Calif.). This process avoids the need to screen libraries and is useful in finding intron/exon junctions.

In order to express biologically active proteins of the invention, the nucleotide sequences encoding the proteins of the invention optionally in the form of fusion proteins, may be inserted into appropriate expression vectors, i.e., a vector, which contains the necessary elements for the transcription and translation of the inserted coding sequence. Methods, which are well known to those skilled in the art, may be used to construct expression vectors containing sequences encoding the proteins of the invention and appropriate transcriptional and translational control elements. These methods include in vitro recombinant DNA techniques. synthetic techniques, and in vivo genetic recombination. Such techniques are described in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y.

Regulatory elements include for example a promoter, an initiation codon, a stop codon, a mRNA stability regulatory element, and a polyadenylation signal. Expression of a polynucleotide can be assured by (i) constitutive promoters such as the Cytomegalovirus (CMV) promoter/enhancer region, (ii) tissue specific promoters such as the insulin promoter (see, Soria et al., 2000, Diabetes 49:157), SOX2 gene promotor (see Li et al., 1998, Curr. Biol. 8:971-4), Msi-1 promotor (see Sakakibara et al., 1997, J. Neuroscience 17:8300-8312), alpha-cardia myosin heavy chain promotor or human atrial natriuretic factor promotor (Klug et al., 1996, J. clin. Invest 98:216-24; Wu et al., 1989, J. Biol. Chem. 264:6472-79) or (iii) inducible promoters such as the tetracycline inducible system. Expression vectors can also contain a selection agent or marker gene that confers antibiotic resistance such as the neomycin, hygromycin or puromycin resistance genes. These methods include in vitro recombinant DNA techniques, synthetic techniques, and in vivo genetic recombination. Such techniques are described in Sambrook, J. et al. (1989) Molecular Cloning, A Laboratory Manual, Cold Spring Harbor Press, Plainview, N.Y. and Ausubel, F. M. et al. (1989) Current Protocols in Molecular Biology, John Wiley & Sons, New York, N.Y. In a further embodiment of the invention, natural, modified or recombinant nucleic acid sequences encoding the proteins of the invention and homologous proteins may be ligated to a heterologous sequence to encode a fusion protein.

A variety of expression vector/host systems may be utilised to contain and express sequences encoding the proteins of the invention or fusion proteins. These include, but are not limited to, micro-organisms such as bacteria transformed with recombinant bacteriophage, plasmid, or cosmid DNA expression vectors; yeast transformed with yeast expression vectors; insect cell systems infected with virus expression vectors (e.g. baculovirus, adenovirus, adeno-associated virus, lentiverus, retrovirus); plant cell systems transformed with virus expression vectors (e.g. cauliflower mosaic virus, CaMV; tobacco mosaic virus, TMV) or with bacterial expression vectors (e.g. Ti or PBR322 plasmids); or animal cell systems.

The presence of polynucleotide sequences encoding the proteins of the invention can be detected by DNA-DNA or DNA-RNA hybridisation or amplification using probes or portions or fragments of polynucleotides encoding the proteins of the invention. Nucleic acid amplification based assays involve the use of oligonucleotides or oligomers based on the sequences encoding the proteins of the invention to detect transformants containing DNA or RNA encoding the proteins of the invention. As used herein “oligonucleotides” or “oligomers” refer to a nucleic acid sequence of at least about 10 nucleotides and as many as about 60 nucleotides, preferably about 15 to 30 nucleotides, and more preferably about 20-25 nucleotides, which can be used as a probe or amplimer.

A wide variety of labels and conjugation techniques are known by those skilled in the art and may be used in various nucleic acid and amino acid assays. Means for producing labelled hybridisation or PCR probes for detecting sequences related to polynucleotides encoding the proteins of the invention include oligo-labelling, nick translation, end-labelling or PCR amplification using a labelled nucleotide, or enzymatic synthesis. These procedures may be conducted using a variety of commercially available kits (Pharmacia & Upjohn, (Kalamazoo, Mich.); Promega (Madison Wis.); and U.S. Biochemical Corp., (Cleveland, Ohio).

The presence of proteins of the invention in a sample can be determined by immunological methods or activity measurement. A variety of protocols for detecting and measuring the expression of proteins, using either polyclonal or monoclonal antibodies specific for the protein or reagents for determining protein activity are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on the protein is preferred, but a competitive binding assay may be employed. These and other assays are described, among other places, in Hampton, R. et al. (1990; Serological Methods, a Laboratory Manual, APS Press, St Paul, Minn.) and Maddox, D. E. et al. (1983; J. Exp. Med. 158:1211-1216).

Suitable reporter molecules or labels, which may be used, include radionuclides, enzymes, fluorescent, chemiluminescent, or chromogenic agents as well as substrates, co-factors, inhibitors, magnetic particles, and the like.

The nucleic acids encoding the proteins of the invention can be used to generate transgenic animal or site specific gene modifications in cell lines. Transgenic animals may be made through homologous recombination, where the normal locus of the genes encoding the proteins of the invention is altered. Alternatively, a nucleic acid construct is randomly integrated into the genome. Vectors for stable integration include plasmids, retrovirusses and other animal virusses, YACs, and the like. The modified cells or animal are useful in the study of the function and regulation of the proteins of the invention. For example, a series of small deletions and/or substitutions may be made in the genes that encode the proteins of the invention to determine the role of particular domains of the protein, functions in pancreatic differentiation, etc.

Specific constructs of interest include anti-sense molecules, which will block the expression of the proteins of the invention, or expression of dominant negative mutations. A detectable marker, such as for example lac-Z, may be introduced in the locus of the genes of the invention, where upregulation of expression of the genes of the invention will result in an easily detected change in phenotype.

One may also provide for expression of the genes of the invention or variants thereof in cells or tissues where it is not normally expressed or at abnormal times of development. In addition, by providing expression of the proteins of the invention in cells in which they are not normally produced, one can induce changes in cell behavior.

DNA constructs for homologous recombination will comprise at least portions of the genes of the invention with the desired genetic modification, and will include regions of homology to the target locus. DNA constructs for random integration need not include regions of homology to mediate recombination. Conveniently, markers for positive and/or negative selection are included. Methods for generating cells having targeted gene modifications through homologous recombination are known in the art. For embryonic stem (ES) cells, an ES cell line may be employed, or embryonic cells may be obtained freshly from a host, e.g. mouse, rat, guinea pig etc. Such cells are grown on an appropriate fibroblast-feeder layer or grown in presence of leukemia inhibiting factor (LIF).

When ES or embryonic cells have been transformed, they may be used to produce transgenic animals. After transformation, the cells are plated onto a feeder layer in an appropriate medium. Cells containing the construct may be detected by employing a selective medium. After sufficient time for colonies to grow, they are picked and analyzed for the occurrence of homologous recombination or integration of the construct. Those colonies that are positive may then be used for embryo manipulation and blastocyst injection. Blastocysts are obtained from 4 to 6; week old superovulated females. The ES cells are trypsinized, and the modified cells are injected into the blastocoel of the blastocyst. After injection, the blastocysts are returned to each uterine horn of pseudopregnant females. Females are then allowed to go to term and the resulting offspring screened for the construct. By providing for a different phenotype of the blastocyst and the genetically modified cells, chimeric progeny can be readily detected. The chimeric animals are screened for the presence of the modified gene and males and females having the modification are mated to produce homozygous progeny. If the gene alterations cause lethality at some point in development, tissues or organs can be maintained as allogenic or congenic grafts or transplants, or in vitro culture. The transgenic animals may be any non-human mammal, such as laboratory animal, domestic animals, etc. The transgenic animals may be used in functional studies, drug screening, etc.

Diagnostics and Therapeutics

The data disclosed in this invention show that the nucleic acids and proteins of the invention and effector molecules thereof are useful in diagnostic and therapeutic applications implicated, for example but not limited to, in metabolic disorders such as obesity as well as related disorders such as eating disorder, cachexia, diabetes mellitus, hypertension, coronary heart disease, hypercholesterolemia, dyslipidemia, osteoarthritis, gallstones, cancer, e.g. cancers of the reproductive organs, and sleep apnea. Hence, diagnostic and therapeutic uses for the nucleic acids and proteins of the invention and effectors thereof are, for example but not limited to, the following: (i) protein therapeutic, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues).

The nucleic acids and proteins of the invention and effectors thereof are useful in diagnostic and therapeutic applications implicated in various applications as described below. For example, but not limited to, cDNAs encoding the proteins of the invention and particularly their human homologues may be useful in gene therapy, and the proteins of the invention and particularly their human homologues may be useful when administered to a subject in need thereof. By way of non-limiting example, the compositions of the present invention will have efficacy for treatment of patients suffering from, for example, but not limited to, in metabolic disorders as described above.

The nucleic acids encoding the proteins of the invention, or fragments thereof, may further be useful in diagnostic applications, wherein the presence or amount of the nucleic acids or the proteins are to be assessed. These materials are further useful in the generation of antibodies that bind immunospecifically to the substances of the invention for use in therapeutic or diagnostic methods.

For example, in one aspect, antibodies which are specific for the proteins of the invention may be used directly as an antagonist, or indirectly as a targeting or delivery mechanism for bringing a pharmaceutical agent to cells or tissue which express the proteins of the invention. The antibodies may be generated using methods that are well known in the art. Such antibodies may include, but are not limited to, polyclonal, monoclonal, chimerical, single chain, Fab fragments, and fragments produced by a Fab expression library. Neutralising antibodies, (i.e., those which inhibit dimer formation) are especially preferred for therapeutic use.

For the production of antibodies, various hosts including goats, rabbits, rats, mice, humans, and others, may be immunised by injection with the proteins of the invention any fragment or oligopeptide thereof which has immunogenic properties. Depending on the host species, various adjuvants may be used to increase immunological response. It is preferred that the peptides, fragments, or oligopeptides used to induce antibodies to the proteins of the invention have an amino acid sequence consisting of at least five amino acids, and more preferably at least 10 amino acids.

Monoclonal antibodies to the proteins of the invention may be prepared using any technique which provides for the production of antibody molecules by continuous cell lines in culture. These include, but are not limited to, the hybridoma technique, the human B-cell hybridoma technique, and the EBV-hybridoma technique, (Kohler, G. et al. (1975) Nature 256:495-497; Kozbor, D. et al. (1985) J. Immunol. Methods 81:31-42; Cote, R. J. et al. Proc. Natl. Acad. Sci. 80:2026-2030; Cole, S. P. et al. (1984) Mol. Cell Biol. 62:109-120).

In addition, techniques developed for the production of “chimeric antibodies”, the splicing of mouse antibody genes to human antibody genes to obtain a molecule with appropriate antigen specificity and biological activity can be used (Morrison, S. L. et al. (1984) Proc. Natl. Acad. Sci. 81:6851-6855; Neuberger, M. S. et al (1984) Nature 312:604-608; Takeda, S. et al. (1985) Nature 314:452-454). Alternatively, techniques described for the production of single chain antibodies may be adapted, using methods known in the art, to produce the proteins of the invention—and -specific single chain antibodies. Antibodies with related specificity, but of distinct idiotypic composition, may be generated by chain shuffling from random combinatorial immunoglobulin libraries (Burton, D. R. (1991) Proc. Natl. Acad. Sci. 88:11120-3). Antibodies may also be produced by inducing in vivo production in the lymphocyte population or by screening recombinant immunoglobulin libraries or panels of highly specific binding reagents as disclosed in the literature (Orlandi, R. et al. (1989) Proc. Natl. Acad. Sci. 86:3833-3837; Winter, G. et al. (1991) Nature 349:293-299).

Antibody fragments, which contain specific binding sites for the proteins of the invention, may also be generated. For example, such fragments include, but are not limited to, the F(ab′)₂ fragments which can be produced by Pepsin digestion of the antibody molecule and the Fab fragments which can be generated by reducing the disulfide bridges of F(ab′)₂ fragments. Alternatively, Fab expression libraries may be constructed to allow rapid and easy identification of monoclonal Fab fragments with the desired specificity (Huse, W. D. et al. (1989) Science 254:1275-1281).

Various immunoassays may be used for screening to identify antibodies having the desired specificity. Numerous protocols for competitive binding and immunoradiometric assays using either polyclonal or monoclonal antibodies with established specificities are well known in the art. Such immunoassays typically involve the measurement of complex formation between the proteins of the invention and their specific antibodies. A two-site, monoclonal-based immunoassay utilising monoclonal antibodies reactive to two non-interfering epitopes of a protein of the invention is preferred, but a competitive binding assay may also be employed (Maddox, supra).

In another embodiment of the invention, the polynucleotides or fragments thereof, or nucleic acid effector molecules such as antisense molecules, aptamers, RNAi molecules or ribozymes may be used for therapeutic purposes. In one aspect, aptamers, i.e. nucleic acid molecules, which are capable of binding to a protein of the, invention and modulating its activity may be generated by a screening and selection procedure involving the use of combinatorial nucleic acid libraries.

In a further aspect, antisense molecules to the polynucleotide encoding the proteins of the invention may be used in situations in which it would be desirable to block the transcription of the mRNA. In particular, cells may be transformed with sequences complementary to polynucleotides encoding the proteins of the invention. Thus, antisense molecules may be used to modulate the activity of the proteins of the invention, or to achieve regulation of gene function. Such technology is now well know in the art, and sense or antisense oligomers or larger fragments, can be designed from various locations along the coding or control regions of sequences encoding the proteins of the invention. Expression vectors derived from retroviruses, adenovirus, herpes or vaccinia viruses, or from various bacterial plasmids may be used for delivery of nucleotide sequences to the targeted organ, tissue or cell population. Methods, which are well known to those skilled in the art, can be used to construct recombinant vectors, which will express antisense molecules complementary to the polynucleotides of the genes encoding the proteins of the invention. These techniques are described both in Sambrook et al. (supra) and in Ausubel et al. (supra). Genes encoding the proteins of the invention can be turned off by transforming a cell or tissue with expression vectors which express high levels of polynucleotide or fragment thereof which encodes the proteins of the invention. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector and even longer if appropriate replication elements are part of the vector system.

As mentioned above, modifications of gene expression can be obtained by designing antisense molecules, e.g. DNA, RNA, or nucleic acid analogues such as PNA, to the control regions of the gene encoding the proteins of the invention, i.e., the promoters, enhancers, and introns. Oligonucleotides derived from the transcription initiation site, e.g. between positions −10 and +10 from the start site, are preferred. Similarly, inhibition can be achieved using “triple helix” base-pairing methodology. Triple helix pairing is useful because it cause inhibition of the ability of the double helix to open sufficiently for the binding of polymerases, transcription factors, or regulatory molecules. Recent therapeutic advances using triplex DNA have been described in the literature (Gee, J. E. et al. (1994) In; Huber, B. E. and B. I. Carr, Molecular and Immunologic Approaches, Futura Publishing Co., Mt. Kisco, N.Y.). The antisense molecules may also be designed to block translation of mRNA by preventing the transcript from binding to ribosomes.

Ribozymes, enzymatic RNA molecules, may also be used to catalyse the specific cleavage of RNA. The mechanism of ribozyme action involves sequence-specific hybridisation of the ribozyme molecule to complementary target RNA, followed by endonucleolytic cleavage. Examples, which may be used, include engineered hammerhead motif ribozyme molecules that can be specifically and efficiently catalyse endonucleolytic cleavage of sequences encoding the proteins of the invention. Specific ribozyme cleavage sites within any potential RNA target are initially identified by scanning the target molecule for ribozyme cleavage sites which include the following sequences: GUA, GUU, and GUC. Once identified, short RNA sequences of between 15 and 20 ribonucleotides corresponding to the region of the target gene containing the cleavage site may be evaluated for secondary structural features which may render the oligonucleotide inoperable. The suitability of candidate targets may also be evaluated by testing accessibility to hybridisation with complementary oligonucleotides using ribonuclease protection assays.

Nucleic acid effector molecules, e.g. antisense molecules and ribozymes of the invention may be prepared by any method known in the art for the synthesis of nucleic acid molecules. These include techniques for chemically synthesising oligonucleotides such as solid phase phosphoramidite chemical synthesis. Alternatively, RNA molecules may be generated by in vitro and in vivo transcription of DNA sequences encoding the proteins of the invention. Such DNA sequences may be incorporated into a variety of vectors with suitable RNA polymerase promoters such as T7 or SP6. Alternatively, these cDNA constructs that synthesise antisense RNA constitutively or inducibly can be introduced into cell lines, cells, or tissues. RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine, and wybutosine, as well as acetyl-, methyl-, thio-, and similarly modified forms of adenine, cytidine, guanine, thymine, and uridine which are not as easily recognised by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection and by liposome injections may be achieved using methods, which are well known in the art. Any of the therapeutic methods described above may be applied to any suitable subject including, for example, mammals such as dogs, cats, cows, horses, rabbits, monkeys, and most preferably, humans.

An additional embodiment of the invention relates to the administration of a pharmaceutical composition, in conjunction with a pharmaceutically acceptable carrier, for any of the therapeutic effects discussed above. Such pharmaceutical compositions may consist of the proteins of the invention, antibodies to the proteins of the invention, mimetics, agonists, antagonists, or inhibitors of the proteins of the invention. The compositions may be administered alone or in combination with at least one other agent, such as stabilising compound, which may be administered in any sterile, biocompatible pharmaceutical carrier, including, but not limited to, saline, buffered saline, dextrose, and water. The compositions may be administered to a patient alone, or in combination with other agents, drugs or hormones. The pharmaceutical compositions utilised in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries, which facilitate processing of the active compounds into preparations which, can be used pharmaceutically. Further details on techniques for formulation and administration may be found in the latest edition of Remington's Pharmaceutical Sciences (Maack Publishing Co., Easton, Pa.). Pharmaceutical compositions can be formulated using pharmaceutically acceptable carriers well known in the art in dosages suitable for administration. Such carriers enable the pharmaceutical compositions to be formulated as tablets, pills, dragees, capsules, liquids, gels, syrups, slurries, suspensions, and the like.

The pharmaceutical compositions of the present invention may be manufactured in a manner that is known in the art, e.g. by means of conventional mixing, dissolving, granulating, dragee-making, levigating, emulsifying, encapsulating, entrapping, or lyophilising processes. The pharmaceutical composition may be provided as a salt and can be formed with many acids, including but not limited to, hydrochloric, sulphuric, acetic, lactic, tartaric, malic, succinic, etc. After pharmaceutical compositions have been prepared, they can be placed in an appropriate container and labelled for treatment of an indicated condition. For administration of the proteins of the invention, such labelling would include amount, frequency, and method of administration.

Pharmaceutical compositions suitable for use in the invention include compositions wherein the active ingredients are contained in an effective amount to achieve the intended purpose. The determination of an effective dose is well within the capability of those skilled in the art. For any compounds, the therapeutically effective does can be estimated initially either in cell culture assays, e.g. of preadipocyte cell lines, or in animal models, usually mice, rabbits, dogs, or pigs. The animal model may also be used to determine the appropriate concentration range and route of administration. Such information can then be used to determine useful doses and routes for administration in humans. A therapeutically effective dose refers to that amount of active ingredient, for example the nucleic acids or proteins of the invention or fragments thereof, or antibodies, which is sufficient for treating a specific condition. Therapeutic efficacy and toxicity may be determined by standard pharmaceutical procedures in cell cultures or experimental animals, e.g. ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50. Pharmaceutical compositions, which exhibit large therapeutic indices, are preferred. The data obtained from cell culture assays and animal studies is used in formulating a range of dosage for human use. The dosage contained in such compositions is preferably within a range of circulating concentrations that include the ED50 with little or no toxicity. The dosage varies within this range depending upon the dosage from employed, sensitivity of the patient, and the route of administration. The exact dosage will be determined by the practitioner, in light of factors related to the subject that requires treatment. Dosage and administration are adjusted to provide sufficient levels of the active moiety or to maintain the desired effect. Factors, which may be taken into account, include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation. Normal dosage amounts may vary from 0.1 to 100,000 micrograms, up to a total dose of about 1 g, depending upon the route of administration. Guidance as to particular dosages and methods of delivery is provided in the literature and generally available to practitioners in the art. Those skilled in the art employ different formulations for nucleotides than for proteins or their inhibitors. Similarly, delivery of polynucleotides or polypeptides will be specific to particular cells, conditions, locations, etc.

In another embodiment, antibodies which specifically bind the proteins of the invention may be used for the diagnosis of conditions or diseases characterised by or associated with over- or underexpression of the proteins of the invention, or in assays to monitor patients being treated with the proteins of the invention, agonists, antagonists or inhibitors. The antibodies useful for diagnostic purposes may be prepared in the same manner as those described above for therapeutics. Diagnostic assays for the proteins of the invention include methods, which utilise the antibody and a label to detect the proteins of the invention in human body fluids or extracts of cells or tissues. The antibodies may be used with or without modification, and may be labelled by joining them, either covalently or non-covalently, with a reporter molecule. A wide variety of reporter molecules which are known in the art may be used several of which are described above.

A variety of protocols including ELISA, RIA, and FACS for measuring the proteins of the invention are known in the art and provide a basis for diagnosing altered or abnormal levels of expression of the proteins of the invention. Normal or standard values for expression of the proteins of the invention are established by combining body fluids or cell extracts taken from normal mammalian subjects, preferably human, with antibodies to the proteins of the invention under conditions suitable for complex formation. The amount of standard complex formation may be quantified by various methods, but preferably by photometric means. Quantities of the proteins of the invention expressed in control and disease samples, e.g. from biopsied tissues are compared with the standard values. Deviation between standard and subject values establishes the parameters for diagnosing disease.

In another embodiment of the invention, the polynucleotides specific for the proteins of the invention may be used for diagnostic purposes. The polynucleotides, which may be used, include oligonucleotide sequences, antisense RNA and DNA molecules, and PNAs. The polynucleotides may be used to detect and quantitate gene expression in biopsied tissues in which expression of the proteins of the invention may be correlated with disease. The diagnostic assay may be used to distinguish between absence, presence, and excess expression of the proteins of the invention, and to monitor regulation of the levels of the proteins of the invention during therapeutic intervention.

In one aspect, hybridisation with PCR probes which are capable of detecting polynucleotide sequences, including genomic sequences, encoding the proteins of the invention and homologous proteins or closely related molecules, may be used to identify nucleic acid sequences which encode the respective protein. The hybridisation probes of the subject invention may be DNA or RNA and are preferably derived from the nucleotide sequences of the polynucleotides encoding the Drosophila or human proteins of the invention or from genomic sequence including promoter, enhancer elements, and introns of the naturally occurring gene.

Hybridisation probes may be labelled by a variety of reporter groups, for example, radionuclides such as ³²P or ³⁵S, or enzymatic labels, such as alkaline phosphatase coupled to the probe via avidin/biotin coupling systems, and the like.

Polynucleotide sequences specific for the proteins of the invention and homologous nucleic acids may be used for the diagnosis of conditions or diseases, which are associated with expression of the proteins of the invention. Examples of such conditions or diseases include, but are not limited to, pancreatic diseases and disorders, including diabetes. Polynucleotide sequences specific for the proteins of the invention and homologous proteins may also be used to monitor the progress of patients receiving treatment for pancreatic diseases and disorders, including diabetes. The polynucleotide sequences encoding the proteins of the invention may be used in Southern or Northern analysis, dot blot, or other membrane-based technologies; in PCR technologies; or in dip stick, pin, ELISA or chip assays utilising fluids or tissues from patient biopsies to detect altered gene expression.

In a particular aspect, the nucleotide sequences encoding the proteins of the invention and homologous proteins may be useful in assays that detect activation or induction of various metabolic diseases such as obesity as well as related disorders such as eating disorder, cachexia, diabetes mellitus, hypertension; coronary heart disease, hypercholesterolemia, dyslipidemia, osteoarthritis, gallstones, cancers of the reproductive organs, and sleep apnea. The nucleotide sequences encoding the proteins of the invention may be labelled by standard methods, and added to a fluid or tissue sample from a patient under conditions suitable for the formation of hybridisation complexes. After a suitable incubation period, the sample is washed and the signal is quantitated and compared with a standard value. If the amount of signal in the biopsied or extracted sample is significantly altered from that of a comparable have hybridised with nucleotide sequences in the sample, and the presence of altered levels of nucleotide sequences encoding the proteins of the invention in the sample indicates the presence of the associated disease. Such assays may also be used to evaluate the efficacy of a particular therapeutic treatment regimen in animal studies, in clinical trials, or in monitoring the treatment of an individual patient.

In order to provide a basis for the diagnosis of disease associated with expression of the proteins of the invention, a normal or standard profile for expression is established. This may be accomplished by combining body fluids or cell extracts taken from normal subjects, either animal or human, with sequences, or fragments thereof, which encode the proteins of the invention, under conditions suitable for hybridisation or amplification. Standard hybridisation may be quantified by comparing the values obtained from normal subjects with those from an experiment where a known amount of a substantially purified polynucleotide is used. Standard values obtained from normal samples may be compared with values obtained from samples from patients who are symptomatic for disease. Deviation between standard and subject values is used to establish the presence of disease. Once disease is established and a treatment protocol is initiated, hybridisation assays may be repeated on a regular basis to evaluate whether the level of expression in the patient begins to approximate that, which is observed in the normal patient. The results obtained from successive assays may be used to show the efficacy of treatment over a period ranging from several days to months.

With respect to metabolic diseases such as described above, the presence of an unusual amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the pancreatic diseases and disorders. Additional diagnostic uses for oligonucleotides designed from the sequences encoding the proteins of the invention may involve the use of PCR. Such oligomers may be chemically synthesised, generated enzymatically, or produced from a recombinant source. Oligomers will preferably consist of two nucleotide sequences, one with sense orientation (5prime.fwdarw.3prime) and another with antisense (3prime.rarw.5prime), employed under optimised conditions for identification of a specific gene or condition. The same two oligomers, nested sets of oligomers, or even a degenerate pool of oligomers may be employed under less stringent conditions for detection and/or quantification of closely related DNA or RNA sequences.

In another embodiment of the invention, the nucleic acid sequences, which encode the proteins of the invention, may also be used to generate hybridisation probes, which are useful for mapping the naturally occurring genomic sequence. The sequences may be mapped to a particular chromosome or to a specific region of the chromosome using well known techniques. Such techniques include FISH, FACS, or artificial chromosome constructions, such as yeast artificial chromosomes, bacterial artificial chromosomes, bacterial P1 constructions or single chromosome cDNA libraries as reviewed in Price, C. M. (1993) Blood Rev. 7:127-134, and Trask, B. J. (1991) Trends Genet 7:149-154. FISH (as described in Verma et al. (1988) Human Chromosomes: A Manual of Basic Techniques, Pergamon Press, New York, N.Y.) may be correlated with other physical chromosome mapping techniques and genetic map data. Examples of genetic map data can be found in the 1994 Genome Issue of Science (265:1981f). Correlation between the location of the genes encoding the proteins of the invention on a physical chromosomal map and a specific disease, or predisposition to a specific disease, may help to delimit the region of DNA associated with that genetic disease.

The nucleotide sequences of the subject invention may be used to detect differences in gene sequences between normal, carrier or affected individuals. An analysis of polymorphisms, e.g. single nucleotide polymorphisms may be carried out. Further in situ hybridization of chromosomal preparations and physical mapping techniques such as linkage analysis using established chromosomal markers may be used for extending genetic maps. Often the placement of a gene on the chromosome of another mammalian species, such as mouse, may reveal associated markers even if the number or arm of a particular human chromosome is not known. New sequences can be assigned to chromosomal arms or parts thereof, by physical mapping. This provides valuable information to investigators searching for disease genes using positional cloning or other gene discovery techniques. Once the disease or syndrome has been crudely localized by genetic linkage to a particular genomic region, for example, AT to 11q22-23 (Gatti, R. A. et al. (1988) Nature 336:577-580), any sequences mapping to that area may represent associated or regulatory genes for further investigation. The nucleotide sequences of the subject invention may also be used to detect differences in the chromosomal location due to translocation, inversion, etc. among normal, carrier or affected individuals.

In another embodiment of the invention, the proteins of the invention, their catalytic or immunogenic fragments or oligopeptides thereof, an in vitro model, a genetically altered cell or animal, can be used for screening libraries of compounds in any of a variety of drug screening techniques. One can identify effectors, e.g. receptors, enzymes, proteins, ligands, or substrates that bind to, modulate or mimic the action of one or more of the proteins of the invention. The protein or fragment thereof employed in such screening may be free in solution, affixed to a solid support, borne on a cell surface, or located intracellularly. The formation, of binding complexes, between the proteins of the invention and the agent tested, may be measured. Agents could also, either directly or indirectly, influence the activity of the proteins of the invention. Agents may also interfere with posttranslational modifications of the protein, such as phosphorylation and dephosphorylation, acetylation, alkylation, ubiquitination, proteolytic processing, subcellular localization, or degradation. Moreover, agents could influence the dimerization or oligomerization of the proteins of the invention or, in a heterologous manner, of the proteins of the invention with other proteins, for example, but not exclusively, ion channels, enzymes, receptors, or translation factors. Agents could also act on the physical interaction of the proteins of this invention with other proteins, which are required for protein function, for example, but not exclusively, their downstream signalling. Methods for determining protein-protein Interaction are well known in the art. For example binding of a fluorescently labeled peptide derived from the interacting protein to the protein of the Invention, or vise versa, could be detected by a change in polarisation. In case that both binding partners, which can be either the full length proteins as well as one binding partner as the full length protein and the other just represented as a peptide are fluorescently labeled, binding could be detected by fluorescence energy transfer (FRET) from one fluorophore to the other. In addition, a variety of commercially available assay principles suitable for detection of protein-protein interaction are well known in the art, for example but not exclusively AlphaScreen (PerkinElmer) or Scintillation Proximity Assays (SPA) by Amersham. Alternatively, the interaction of the proteins of the invention with cellular proteins could be the basis for a cell-based screening assay, in which both proteins are fluorescently labeled and interaction of both proteins is detected by analysing cotranslocation of both proteins with a cellular imaging reader, as has been developed for example, but not exclusively, by Cellomics or EvotecOAl. In all cases the two or more binding partners can be different proteins with one being the protein of the invention, or in case of dimerization and/or oligomerization the protein of the invention itself. Proteins of the invention, for which one target mechanism of interest, but not the only one, would be such protein/protein interactions are CSP, F-box, coronin, ABC50, and Sec61 alpha.

Assays for determining enzymatic activity of the proteins of the invention are well known in the art. For example, but not exclusively, the activity of malic enzyme could be determined by monitoring the increase of NADPH concentration during enzymatic reaction by the Beutler assay (Beutler E. (1970) Br. J. Haematol. 18:117-121). GST2 activity could for example be measured by spectrometric methods based on monitoring prostaglandine synthetic activity or, glutathione S-transferase activity (Pinzar et al. (2000) J. Biol. Chem. 275:31239-31244). The GTPase activity of RabR1 and the ATPase activity of VhaPPA1-1 could represent target mechanisms for these enzymes. Examples for addressing posttranslational modification are the palmitoylation and farnesylation of RabRP1 and CSP. In that case, the enzymes mediating such posttranslational modification would be targeted, an approach very well known in the art for the farnesylation of the Ras protein (Prendergast G. C. and Rane N. (2001) Expert Opin Investig Drugs 10(12):2105-2116).

Of particular interest are screening assays for agents that have a low toxicity for mammalian cells. The term “agent” as used herein describes any molecule, e.g. protein or pharmaceutical, with the capability of altering or mimicking the physiological function of one or more of the proteins of the invention. Candidate agents encompass numerous chemical classes, though typically they are organic molecules, preferably small organic compounds having a molecular weight of more than 50 and less than about 2,500 Daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise carbocyclic or heterocyclic structures and/or aromatic or polyaromatic structures substituted with one or more of the above functional groups.

Candidate agents are also found among biomolecules including peptides, saccharides, fatty acids, steroids, purines, pyrimidines, nucleic acids and derivatives, structural analogs or combinations thereof. Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Where the screening assay is a binding assay, one or more of the molecules may be joined to a label, where the label can directly or indirectly provide a detectable signal.

Another technique for drug screening, which may be used, provides for high throughput screening of compounds having suitable binding affinity to the protein of interest as described in published PCT application WO84/03564. In this method, as applied to the proteins of the invention large numbers of different small test compounds, e.g. aptamers, peptides, low-molecular weight compounds etc., are provided or synthesized on a solid substrate, such as plastic pins or some other surface. The test compounds are reacted with the proteins or fragments thereof, and washed. Bound proteins are then detected by methods well known in the art. Purified proteins can also be coated directly onto plates for use in the aforementioned drug screening techniques. Alternatively, non-neutralizing antibodies can be used to capture the peptide and immobilize it on a solid support. In another embodiment, one may use competitive drug screening assays in which neutralizing antibodies capable of binding the protein specifically compete with a test compound for binding the protein. In this manner, the antibodies can be used to detect the presence of any peptide, which shares one or more antigenic determinants with the protein.

The nucleic acids encoding the proteins of the invention can be used to generate transgenic cell lines and animals. These transgenic animals are useful in the study of the function and regulation of the proteins of the invention in vivo. Transgenic animals, particularly mammalian transgenic animals, can serve as a model system for the investigation of many developmental and cellular processes common to humans. Transgenic animals may be made through homologous recombination in embryonic stem cells, where the normal locus of the gene encoding the protein of the invention is mutated. Alternatively, a nucleic acid construct encoding the protein is injected into oocytes and is randomly integrated into the genome. One may also express the genes of the invention or variants thereof in tissues where they are not normally expressed or at abnormal times of development. Furthermore, variants of the genes of the invention like specific constructs expressing anti-sense molecules or expression of dominant negative mutations, which will block or alter the expression of the proteins of the invention may be randomly integrated into the genome. A detectable marker, such as lac Z or luciferase may be introduced into the locus of the genes of the invention, where upregulation of expression of the genes of the invention will result in an easily detectable change in phenotype. Vectors for stable integration include plasmids, retroviruses and other animal viruses, yeast artificial chromosomes (YACs), and the like. DNA constructs for homologous recombination will contain at least portions of the genes of the invention with the desired genetic modification, and will include regions of homology to the target locus. Conveniently, markers for positive and negative selection are included. DNA constructs for random integration do not need to contain regions of homology to mediate recombination. DNA constructs for random integration will consist of the nucleic acids encoding the proteins of the invention, a regulatory element (promoter), an intron and a poly-adenylation signal. Methods for generating cells having targeted gene modifications through homologous recombination are known in the field. For embryonic stem (ES) cells, an ES cell line may be employed, or embryonic cells may be obtained freshly from a host, e.g. mouse, rat, guinea pig, etc. Such cells are grown on an appropriate fibroblast-feeder layer and are grown in the presence of leukemia inhibiting factor (LIF). ES or embryonic cells may be transfected and can then be used to produce transgenic animals. After transfection, the ES cells are plated onto a feeder layer in an appropriate medium. Cells containing the construct may be selected by employing a selection medium. After sufficient time for colonies to grow, they are picked and analyzed for the occurrence of homologous recombination. Colonies that are positive may then be used for embryo manipulation and morula aggregation. Briefly, morulae are obtained from 4 to 6 week old superovulated females, the Zona Pellucida is removed and the morulae are put into small depressions of a tissue culture dish. The ES cells are trypsinized, and the modified cells are placed into the depression closely to the morulae. On the following day the aggregates are transfered into the uterine horns of pseudopregnant females. Females are then allowed to go to term. Chimeric offsprings can be readily detected by a change in coat color and are subsequently screened for the transmission of the mutation into the next generation (F1-generation). Offspring of the F1-generation are screened for the presence of the modified gene and males and females having the modification are mated to produce homozygous progeny. If the gene alterations cause lethality at some point in development, tissues or organs can be maintained as allogenic or congenic grafts or transplants, or in vitro culture. The transgenic animals may be any non-human mammal, such as laboratory animal, domestic animals, etc., for example, mouse, rat, guinea pig, sheep, cow, pig, and others. The transgenic animals may be used in functional studies, drug screening, and other applications and are useful in the study of the function and regulation of the proteins of the invention in vivo.

Finally, the invention also relates to a kit comprising at least one of
(a) a nucleic acid molecule encoding one of the proteins of the invention or a fragment thereof;
(b) a vector comprising the nucleic acid of (a);
(c) a host cell comprising the nucleic acid of (a) or the vector of (b);
(d) a polypeptide encoded by the nucleic acid of (a);
(e) a fusion polypeptide encoded by the nucleic acid of (a);
(f) an antibody, an aptamer or another receptor against the nucleic acid of (a) or the polypeptide of (d) or (e) and
(g) an anti-sense oligonucleotide of the nucleic acid of (a).

The kit may be used for diagnostic or therapeutic purposes or for screening applications as described above. The kit may further contain user instructions.

The Figures show:

FIG. 1 shows the increase of triglyceride content of HD-EP(3)31178 and HD-EP(3)37100 Drosophila Men mutant caused by homozygous viable integration of the P-vector (in comparison to controls without integration of this vector).

FIG. 2 shows the molecular organisation of the mutated Men protein gene locus.

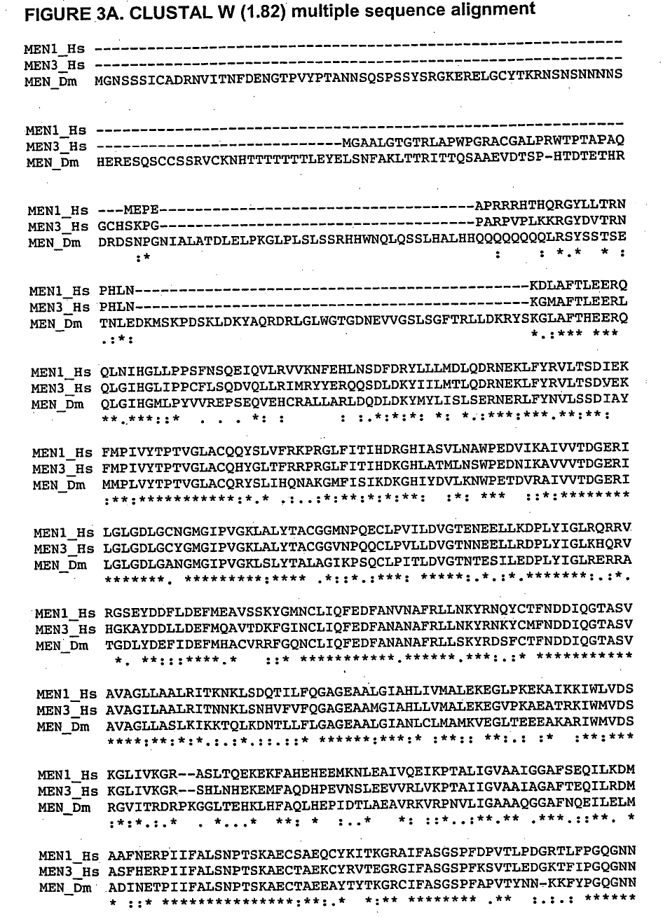

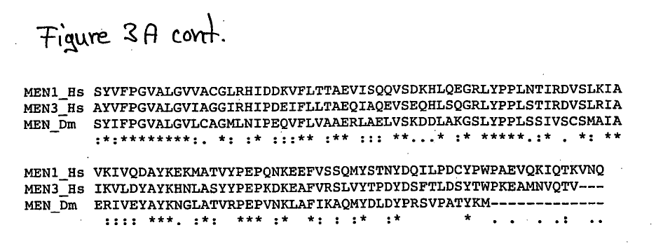

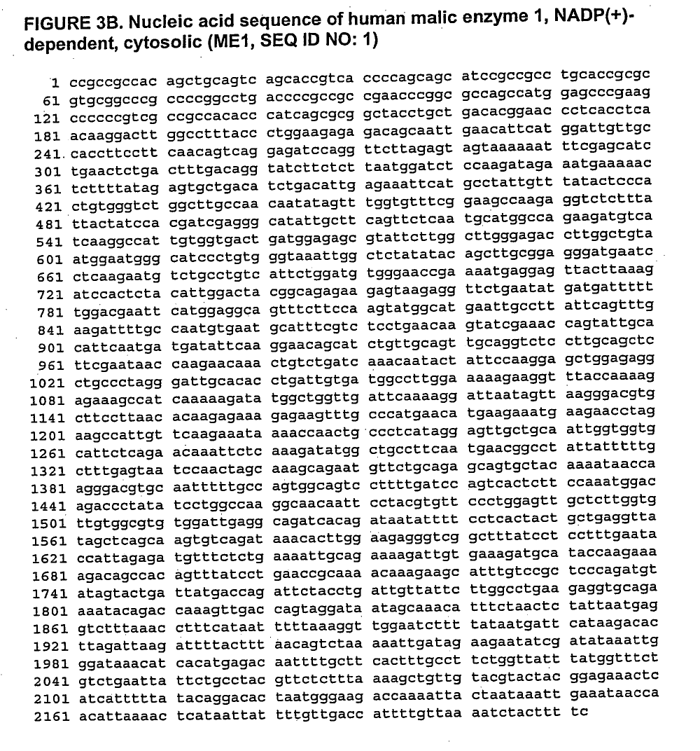

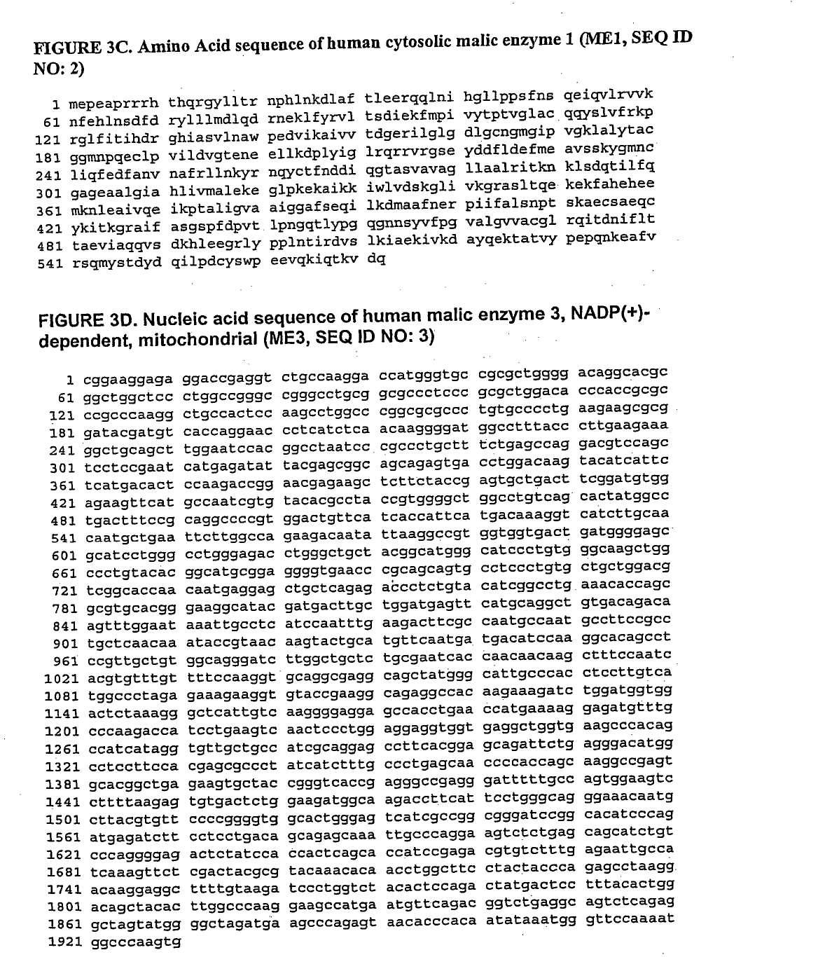

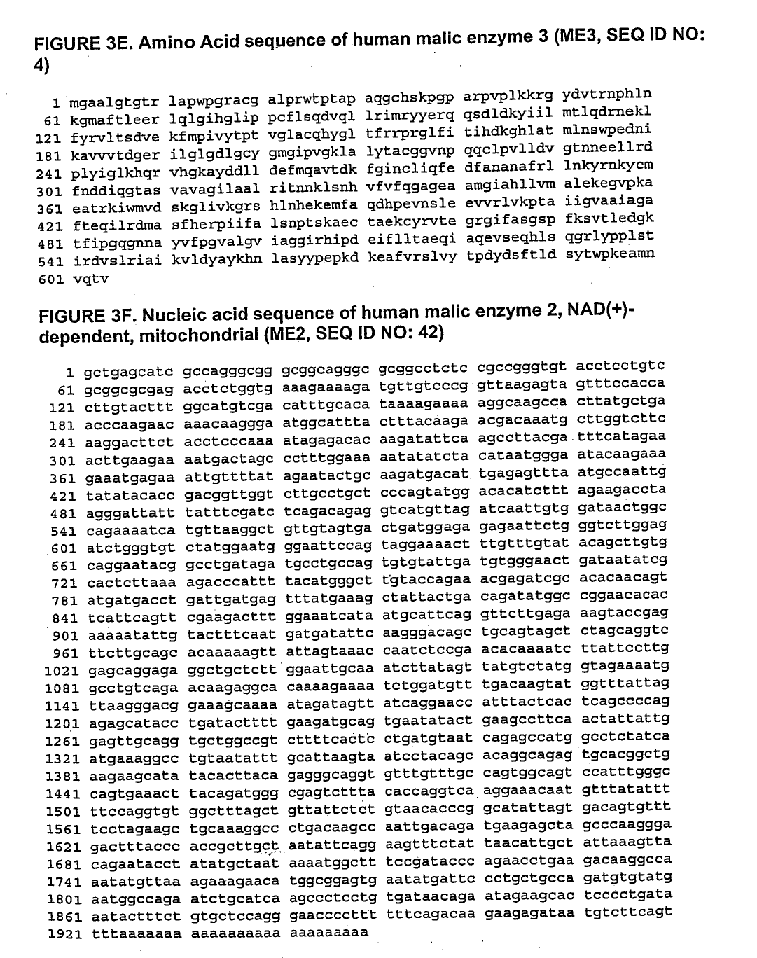

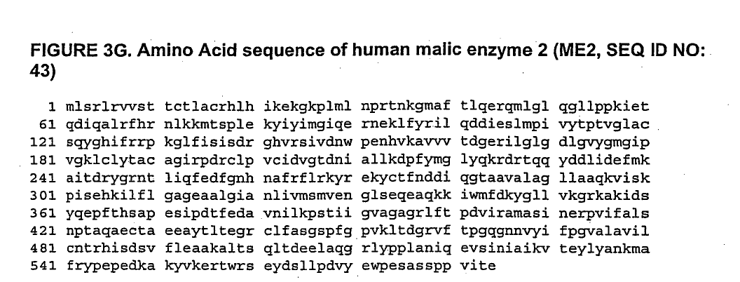

FIG. 3A shows the comparison (CLUSTAL W 1.82 multiple sequence alignment) of Men proteins from different species, MEN1_Hs refers to human malic enzyme 1 (GenBank Accession No. NP_{—}002386), MEN3_Hs refers to human malic enzyme 3 (GenBank Accession No. NP_{—}006671), and MEN_Dm refers to the protein encoded by Drosophila Men gene with GadFly Accession No. CG10120. Gaps in the alignment are represented as -.

FIG. 3B shows the nucleic acid sequence of human malic enzyme 1 (SEQ ID NO: 1).

FIG. 3C shows the amino acid sequence (one-letter code) of human malic enzyme 1 (SEQ ID NO: 2).

FIG. 3D shows the nucleic acid sequence of human malic enzyme 3 (SEQ ID NO: 3).

FIG. 3E shows the amino acid sequence (one-letter code) of human malic enzyme 3 (SEQ ID NO: 4).

FIG. 3F shows the nucleic acid sequence of human malic enzyme 2 (SEQ ID NO: 42).

FIG. 3G shows the amino acid sequence (one-letter code) of human malic enzyme 2 (SEQ ID NO: 43).

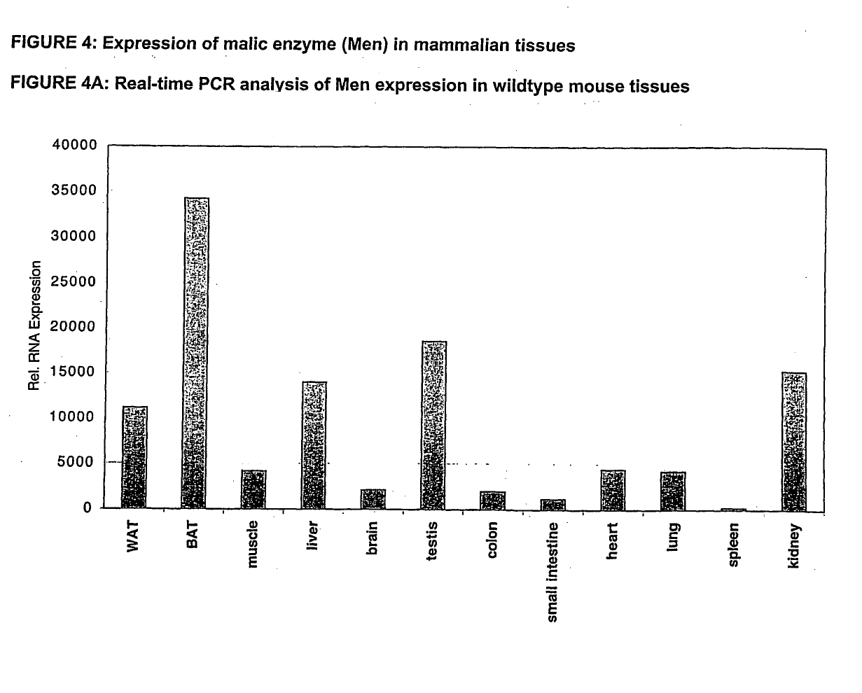

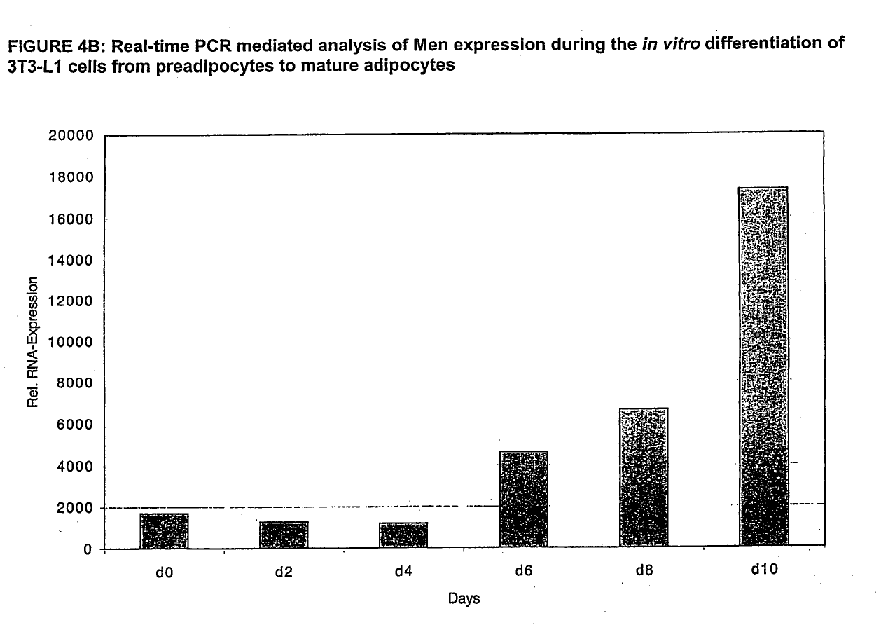

FIG. 4 shows the expression of the Men gene in mammalian tissues.

FIG. 4A shows the real-time PCR analysis of Men in wildtype mouse tissues.

FIG. 4B shows the real-time PCR mediated comparison of Men expression during differentiation of mammalian fibroblast (3T3-L1) cells from pre-adipocytes to mature adipocytes.

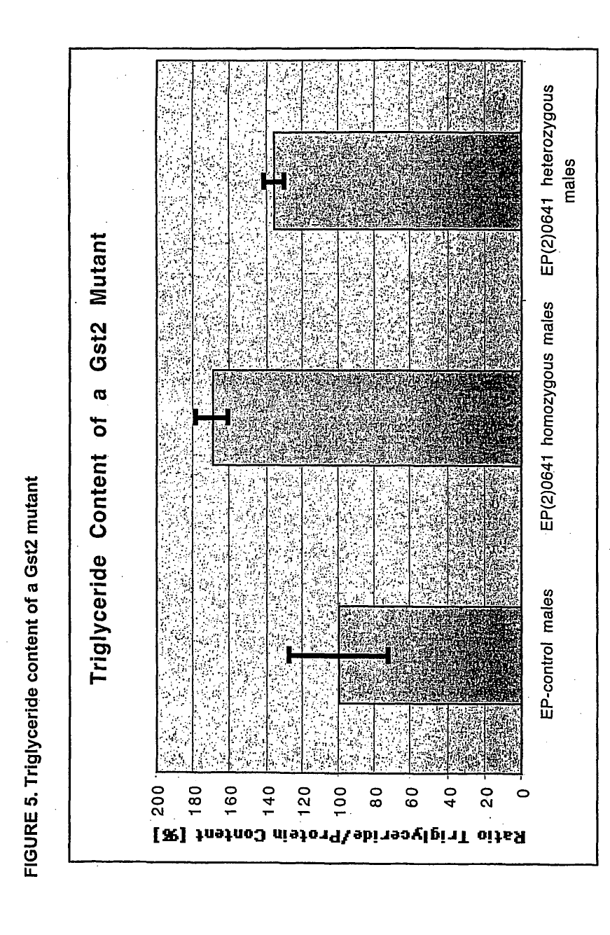

FIG. 5 shows increase of triglyceride content of EP(2)0641 Gst 2 mutant flies caused by homozygous or heterozygous viable integration of the P-vector (in comparison to controls without integration of this vector).

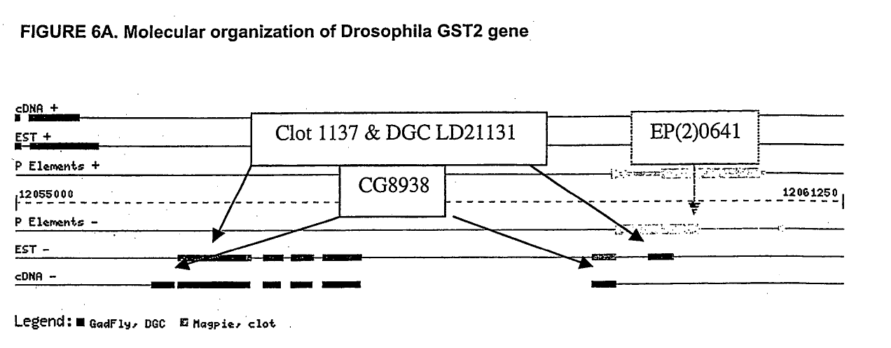

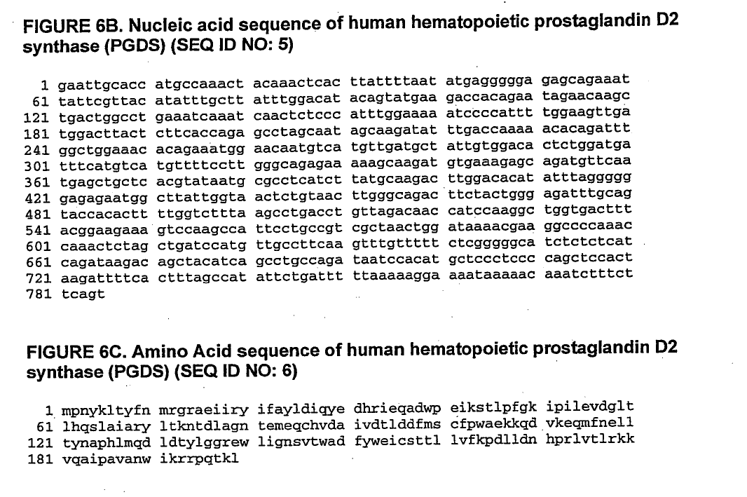

FIG. 6A shows the molecular organisation of the mutated GST2 gene locus.

FIG. 6B shows the nucleic acid sequence of human hematopoietic prostaglandin D2 synthase (PGDS) (SEQ ID NO: 5).

FIG. 6C shows the amino acid sequence (one-letter code) of human hematopoietic prostaglandin D2 synthase (PGDS) (SEQ ID NO: 6).

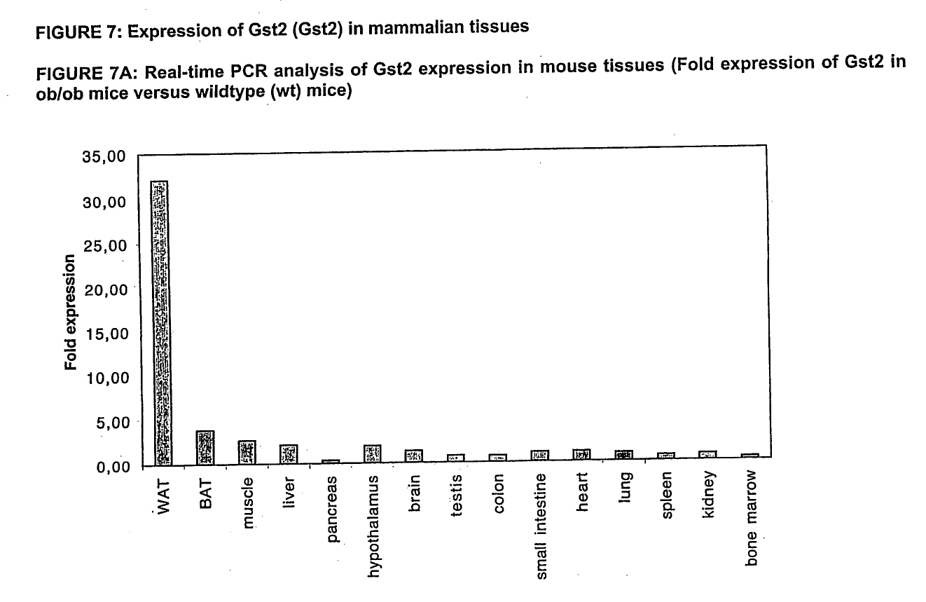

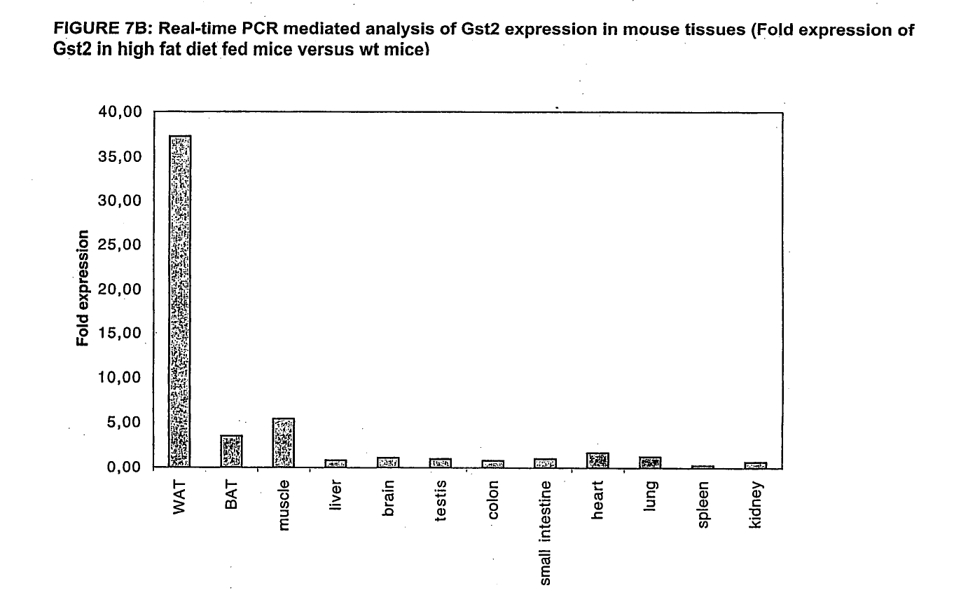

FIG. 7 shows the expression of the Gst2 gene in mammalian tissues.

FIG. 7A shows the real-time PCR analysis of Gst2 expression in ob/ob mice compared with wildtype mouse tissues (shown as fold expression of Gst2 in ob/ob versus wild type mice).

FIG. 7B shows the real-time PCR analysis of Gst2 expression in high fat diet fed mice compared with wildtype mouse tissues (shown as fold expression of Gst2 in ob/ob versus wild type mice).

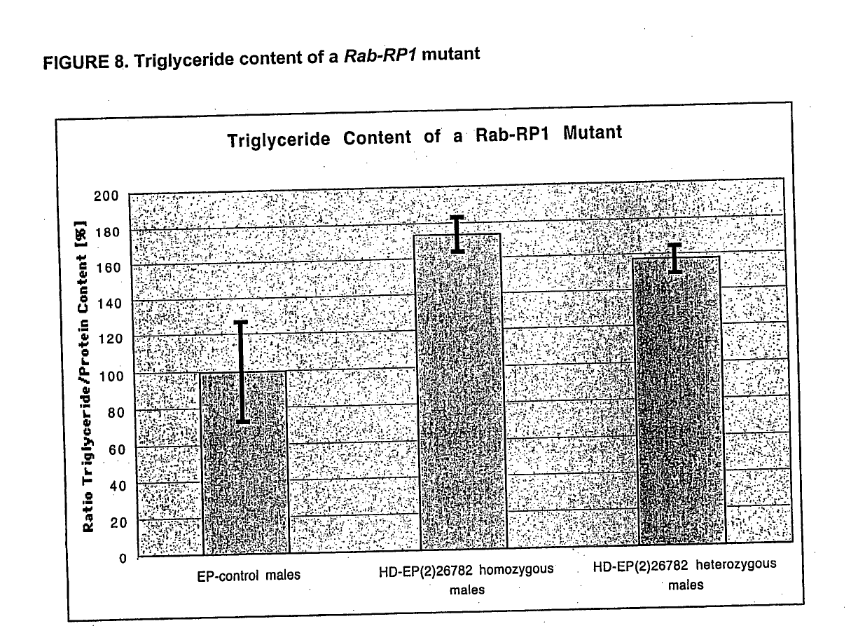

FIG. 8 shows the increase of triglyceride content of HD-EP(2)26782 Rab-RP1 mutant flies caused by homozygous viable or heterozygous integration of the P-vector (in comparison to controls without integration of this vector).

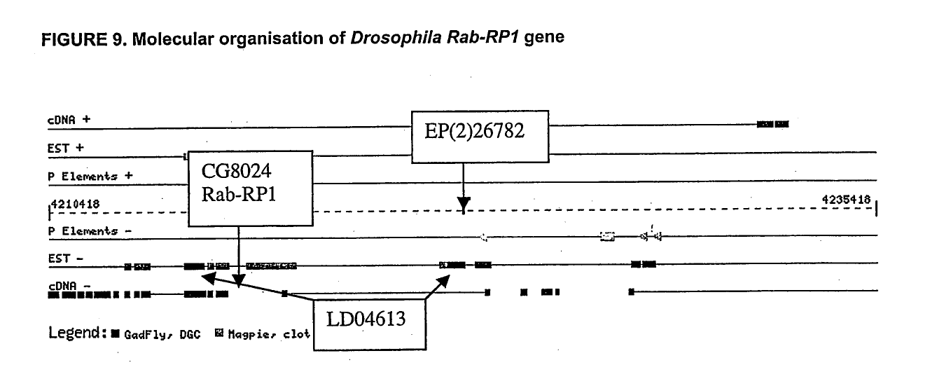

FIG. 9 shows the molecular organisation of the mutated Rab-RP1 gene locus.

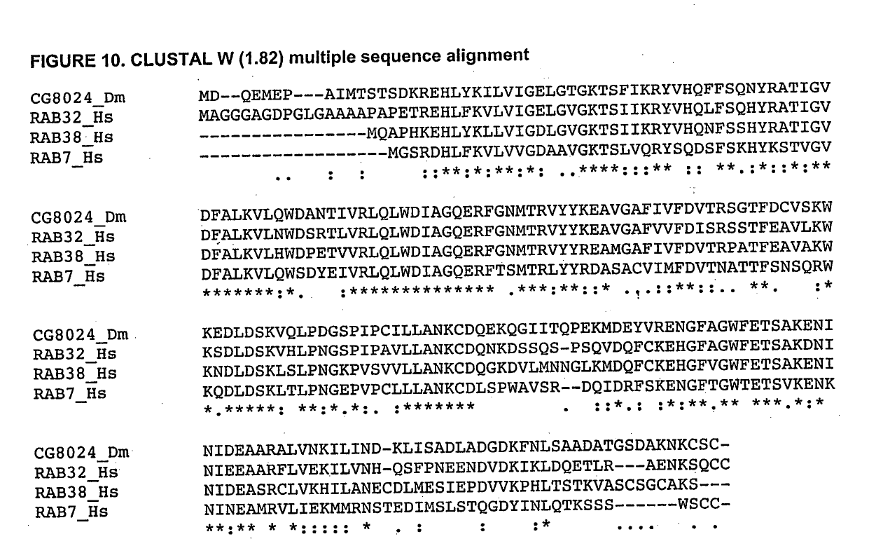

FIG. 10 shows the comparison (CLUSTAL W 1.82 multiple sequence alignment) of Rab proteins from different species, CG8024_Dm refers to the protein encoded by Drosophila Rab-RP1 gene with GadFly Accession No. CG8024, RAB32_Hs refers to human RAB32, member of RAS oncogene family (GenBank Accession No. NP_{—}006825), RAB38_Hs refers to human RAB38, Rab-related GTP-binding protein (GenBank Accession No. NP_{—}071732), and RAB7_Hs refers to human RAB7, member of RAS oncogene family-like 1 (GenBank Accession No. NP_{—}003920). Gaps in the alignment are represented as -.

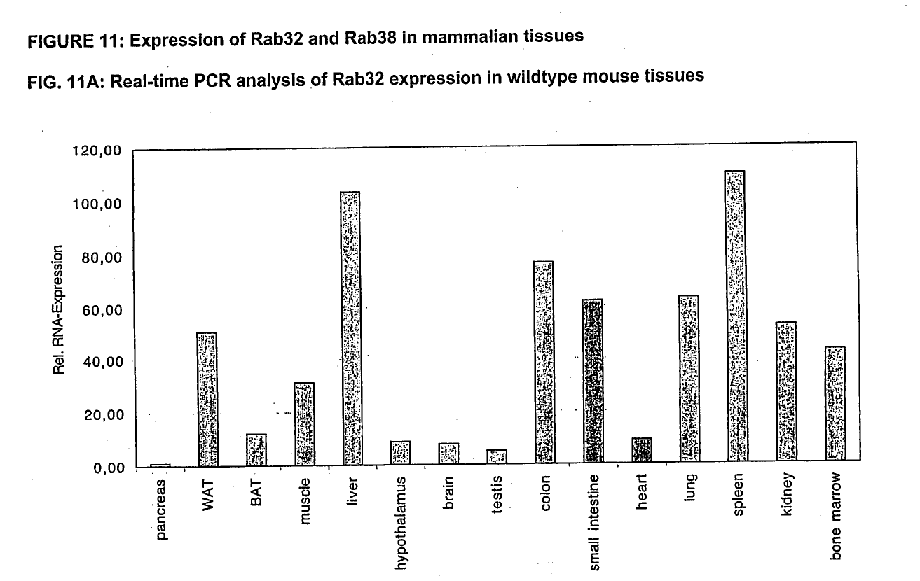

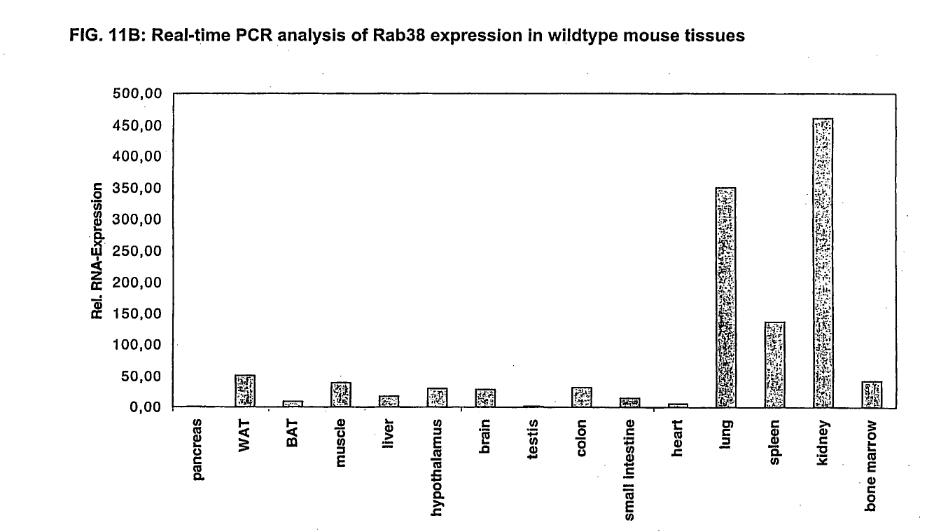

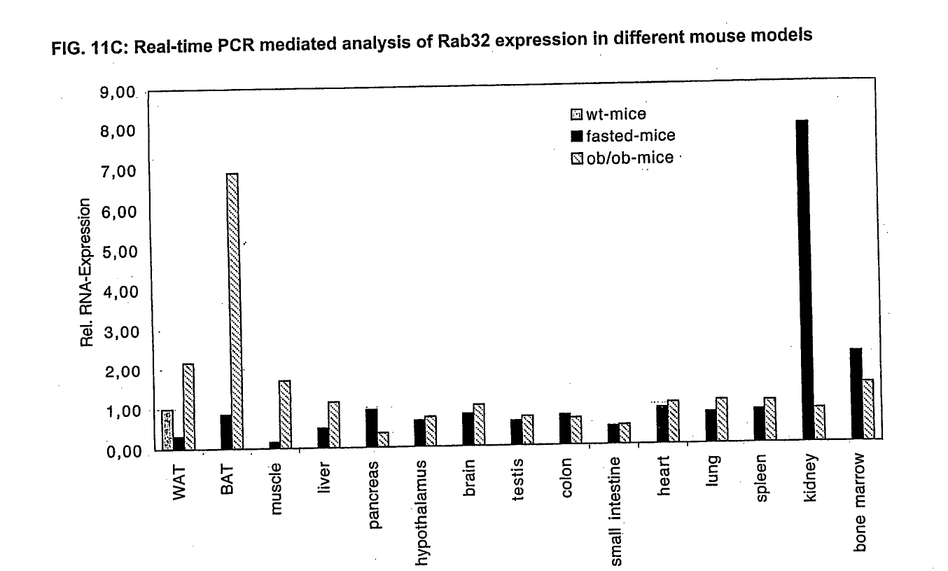

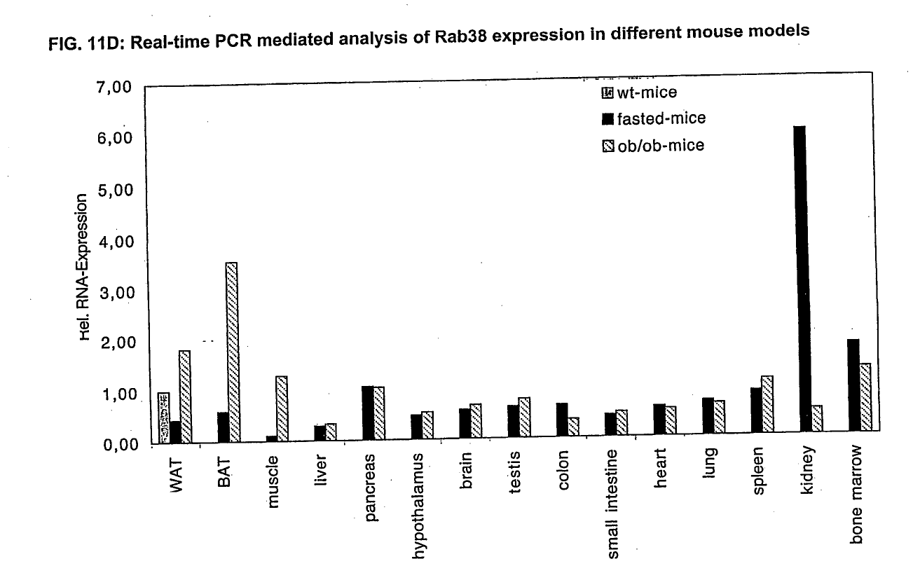

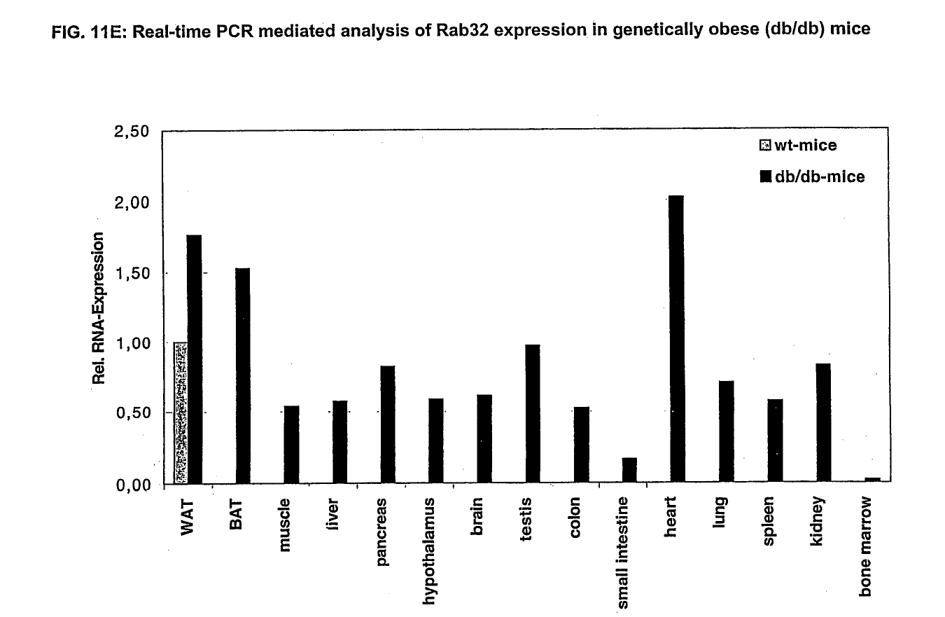

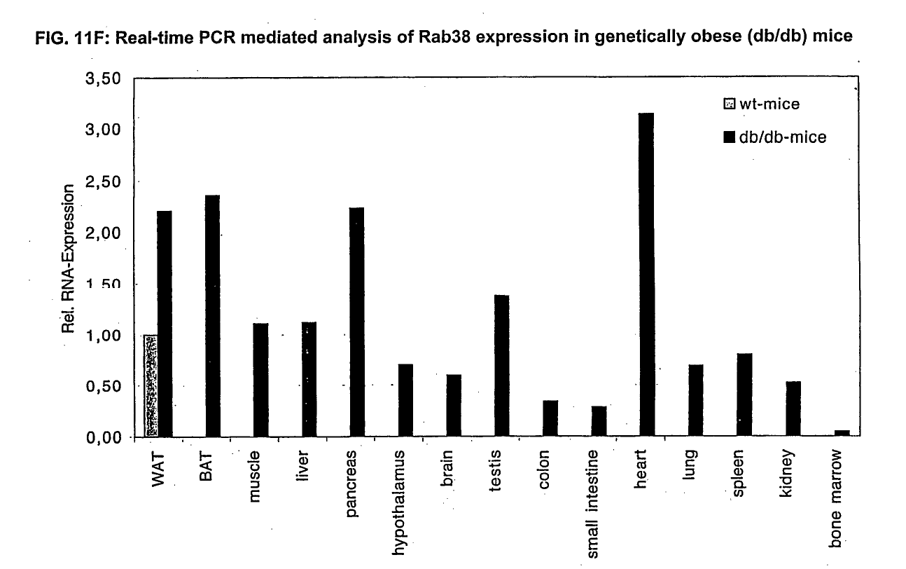

FIG. 11 shows the expression of the Rab32 and Rab38 genes in mammalian tissues.

FIG. 11A shows the real-time PCR analysis of Rab32 in wildtype mouse tissues.

FIG. 11B shows the real-time PCR analysis of Rab38 in wildtype mouse tissues.

FIG. 11C shows the real-time PCR mediated comparison of Rab32 expression in different mouse models.

FIG. 11D shows the real-time PCR mediated comparison of Rab38 expression in different mouse models.

FIG. 11E shows the real-time PCR mediated comparison of Rab32 expression in genetically obese (db/db) and wildtype mice.

FIG. 11F shows the real-time PCR mediated comparison of Rab38 expression in genetically obese (db/db) and wildtype mice.

FIG. 11G shows the real-time PCR mediated comparison of Rab32 expression during differentiation of mammalian fibroblast (3T3-L1) cells from pre-adipocytes to mature adipocytes.

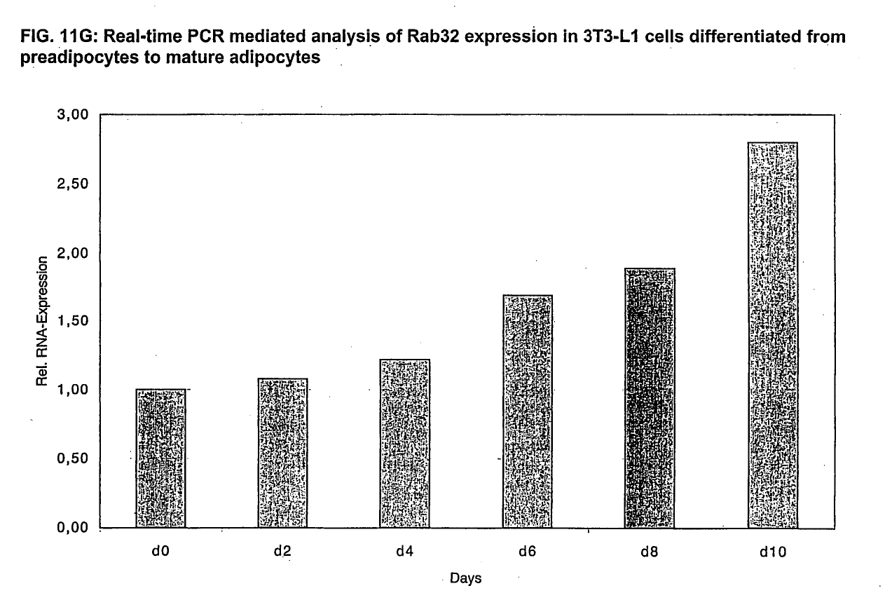

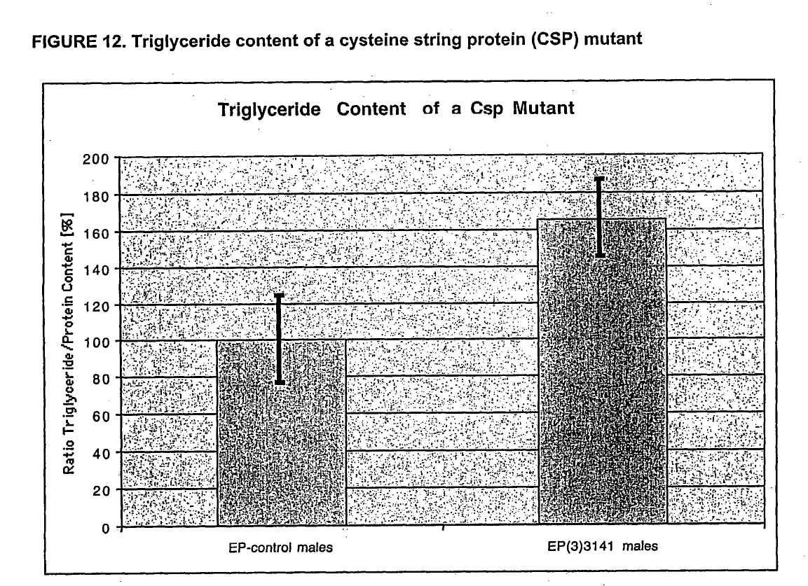

FIG. 12 shows the increase of triglyceride content of EP(3)3141 CSP mutant flies caused by homozygous viable integration of the P-vector (in comparison to controls without integration of this vector).

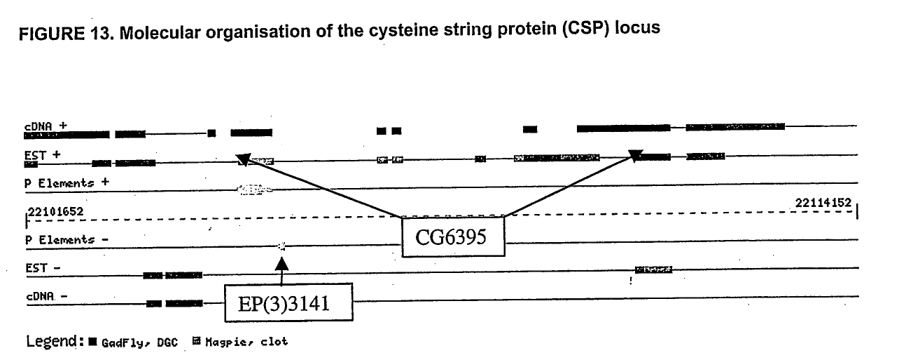

FIG. 13 shows the molecular organisation of the mutated Csp gene locus.

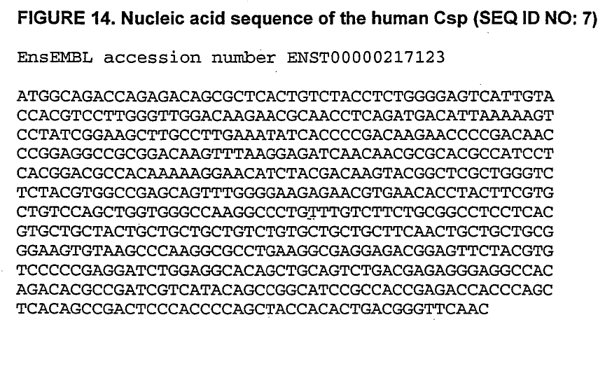

FIG. 14 shows the cDNA sequence of the human Csp (SEQ ID NO: 7).

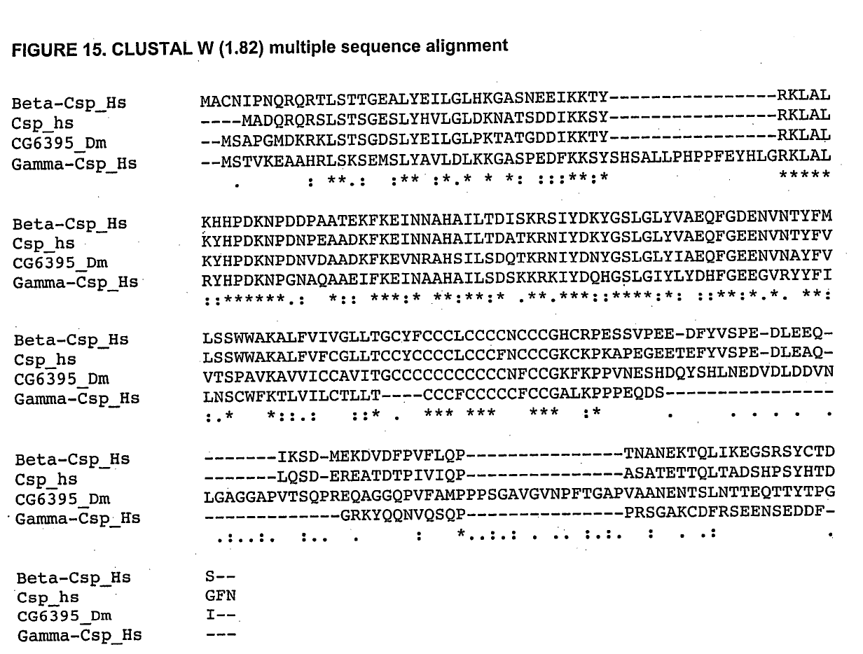

FIG. 15 shows the comparison (CLUSTAL W 1.82 multiple sequence alignment) of Csp proteins from different species, Beta-Csp_Hs refers to human Beta cysteine string protein (GenBank Accession No. Q9UF47), Csp_Hs refers to human cysteine string protein 2 (GenBank Accession No. S70516) CG6395_Dm refers to the protein encoded by Drosophila Csp gene with GadFly Accession No. CG6395, and Gamma-Csp_Hs refers to human unnamed protein product (GenBank Accession No. BAC05155). Gaps in the alignment are represented as -.

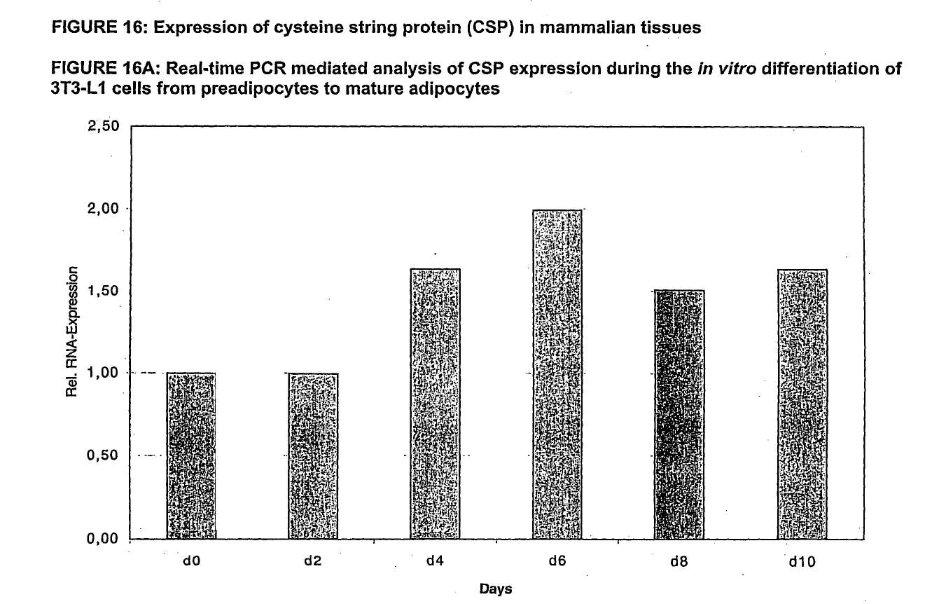

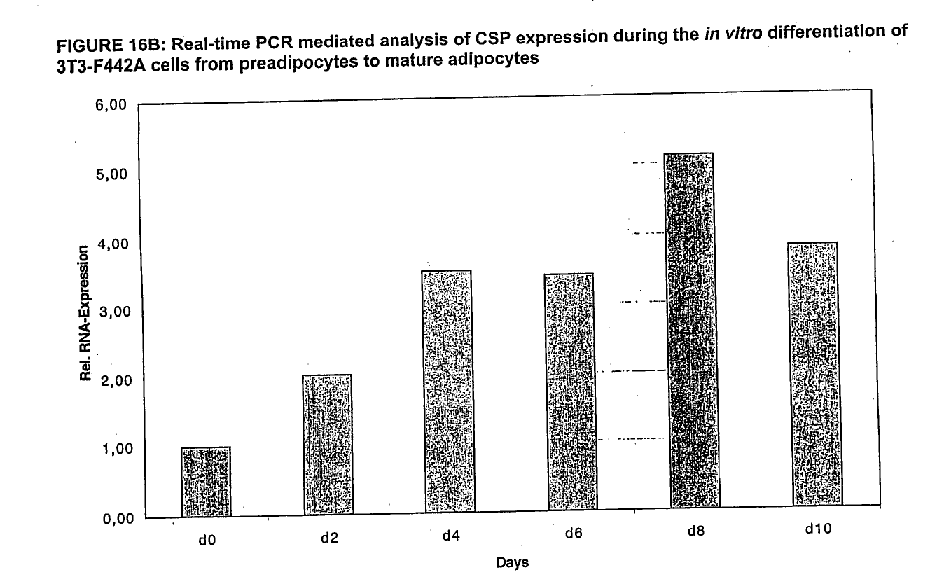

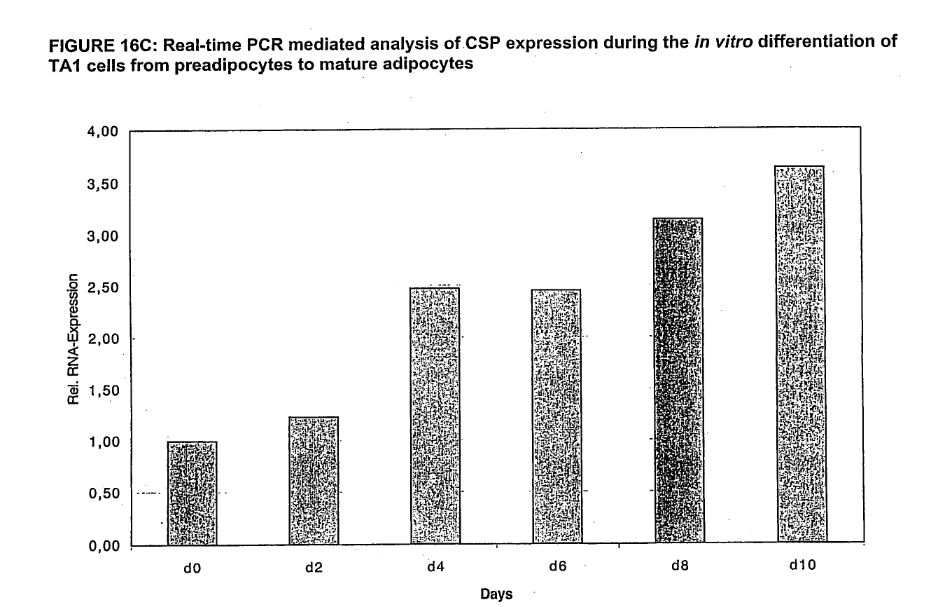

FIG. 16 shows the expression of the Csp gene in mammalian tissues.

FIG. 16A shows the real-time PCR mediated comparison of Csp expression during differentiation of mammalian fibroblast (3T3-L1) cells from pre-adipocytes to mature adipocytes.

FIG. 16B shows the real-time PCR mediated comparison of Csp expression during differentiation of mammalian fibroblast (3T3-F442A) cells from pre-adipocytes to mature adipocytes.

FIG. 16C shows the real-time PCR mediated comparison of Csp expression during differentiation of mammalian TA1 cells from pre-adipocytes to mature adipocytes.

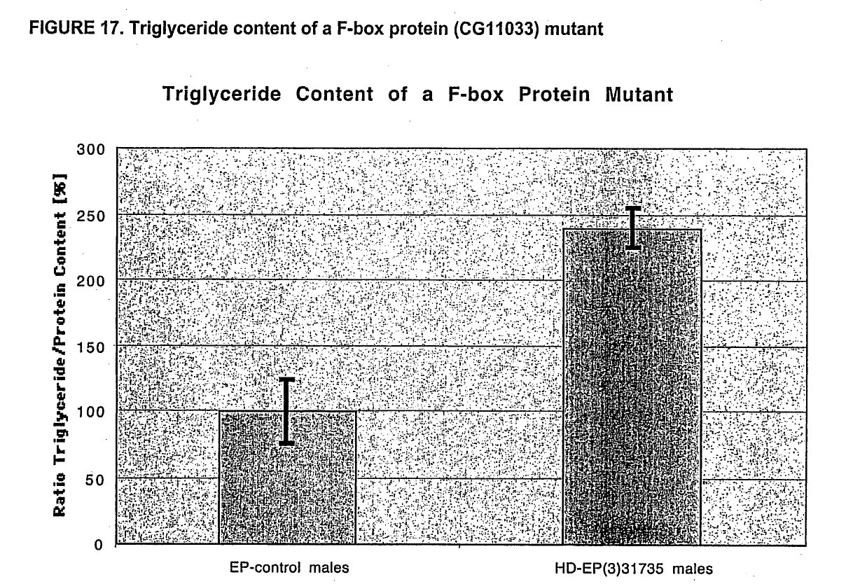

FIG. 17 shows the increase of the triglyceride content of HD-EP(3)31735 F-box mutant flies caused by homozygous viable integration of the P-vector (in comparison to controls without integration of this vector).

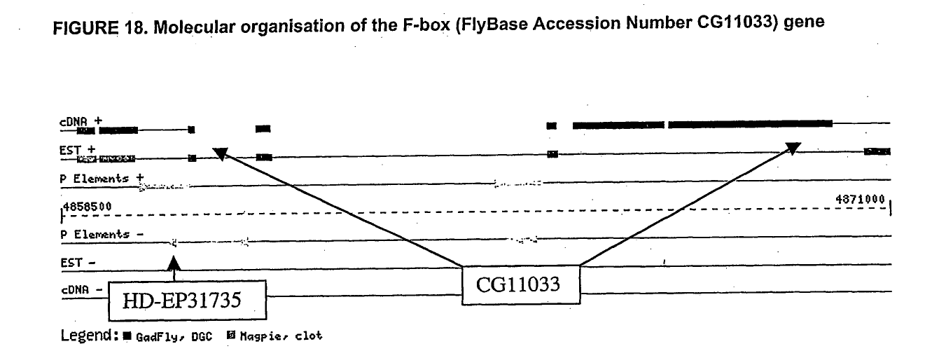

FIG. 18 shows the molecular organisation of the mutated F-box protein Lilina/FBL7 gene locus.

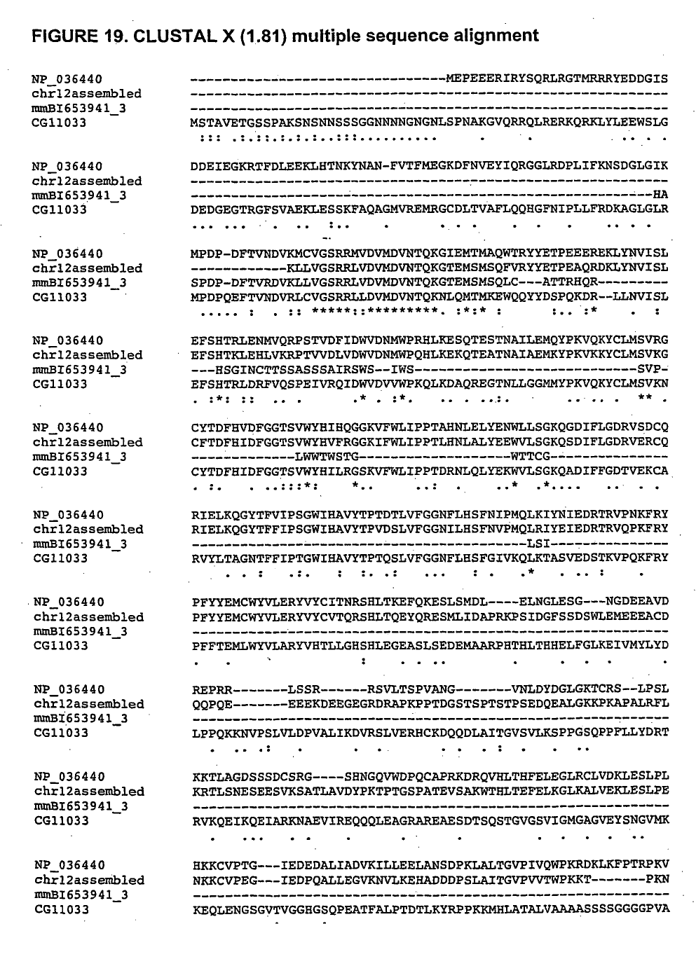

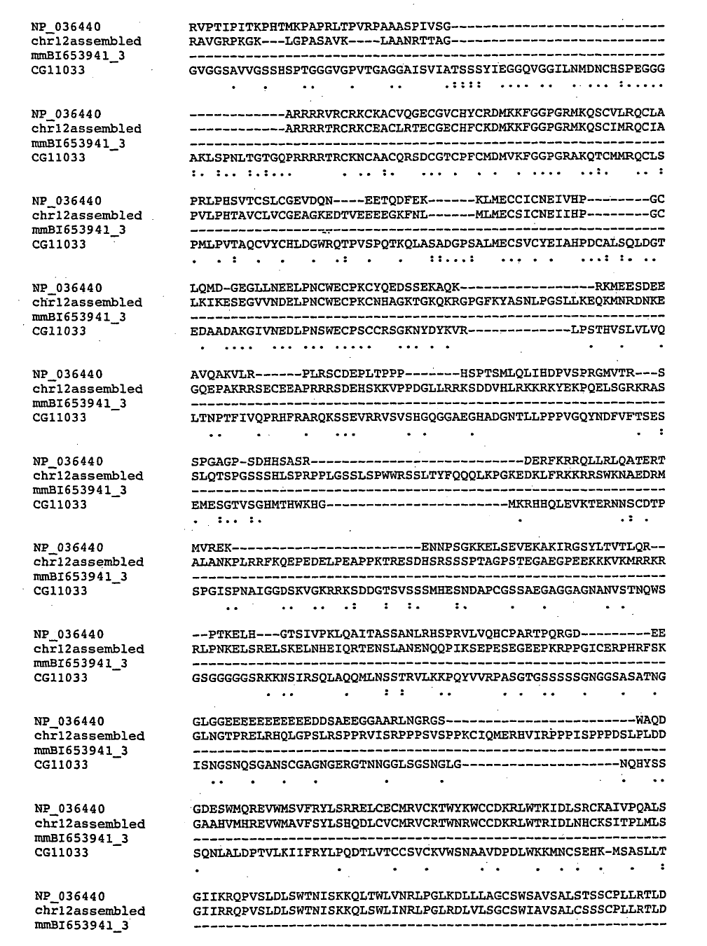

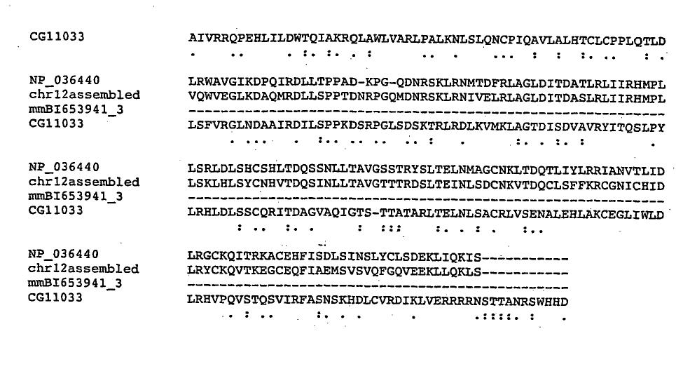

FIG. 19 shows the Clustal X (1.81) multiple sequence alignment. NP_{—}036440 refers to the human F-box protein of the invention, chr12assembled refers to an assembled version of the F-box protein with high homologies to CG11033, mmBI653941_{—}3 refers to the mouse homolog, and CG11033 refers to the Drosophila F-box protein of the invention.

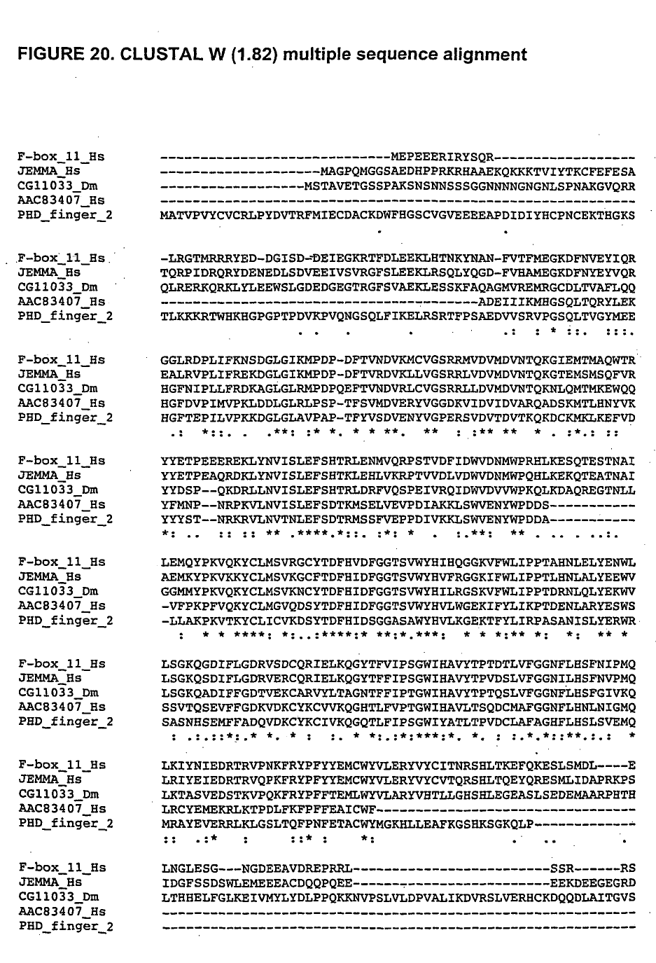

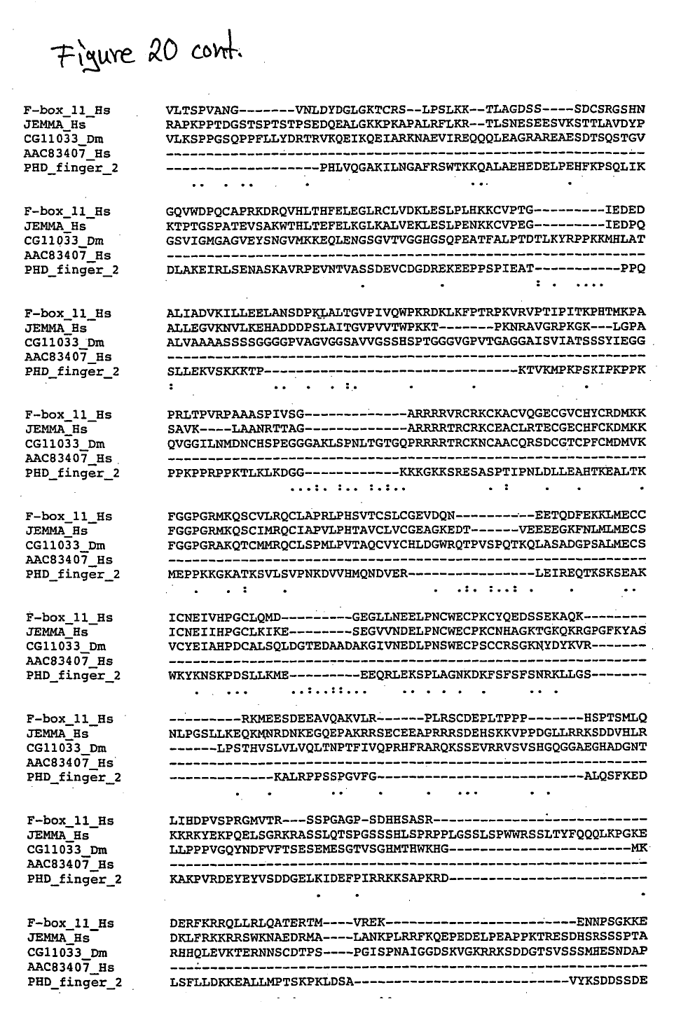

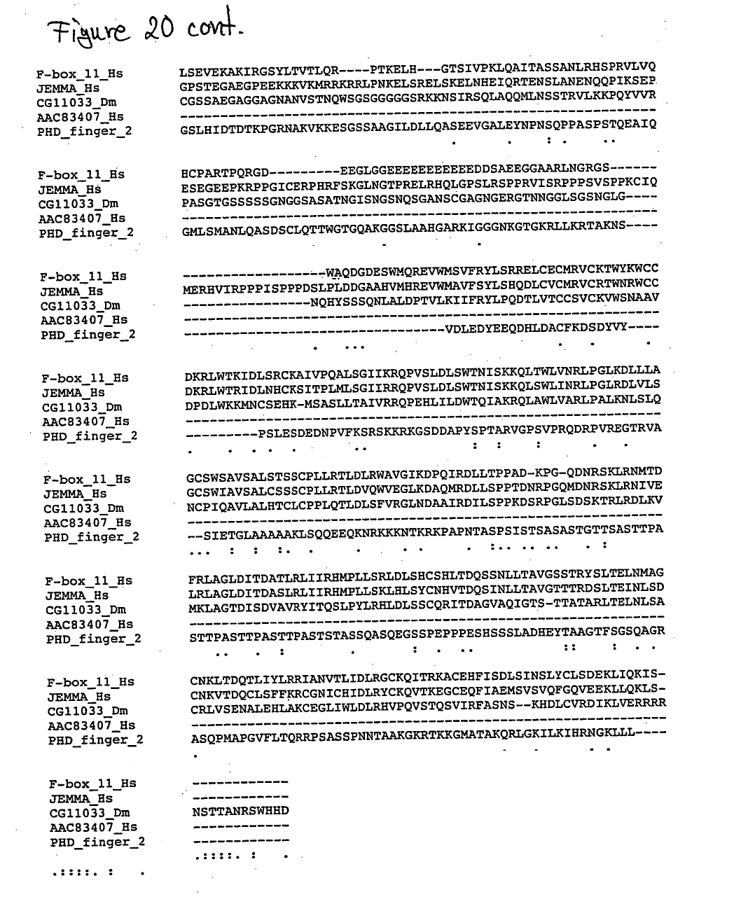

FIG. 20 shows the comparison (CLUSTAL W 1.82 multiple sequence alignment) of F-box proteins from different species, F-box_{—}11_Hs refers to human F-box and leucine rich repeat protein 11 (GenBank Accession No. NP_{—}036440), JEMMA_Hs refers to human JEMMA protein (GenBank Accession No. CAD30700), CG11033_Dm refers to the protein encoded by Drosophila gene with GadFly Accession No. CG11033, AAC83407_Hs refers to human protein similar to several hypothetical proteins (GenBank Accession No. AAC83407), and PHD_finger_{—}2 refers to human PHD finger protein 2 (GenBank Accession No. NP_{—}005383). Gaps in the alignment are represented as -.

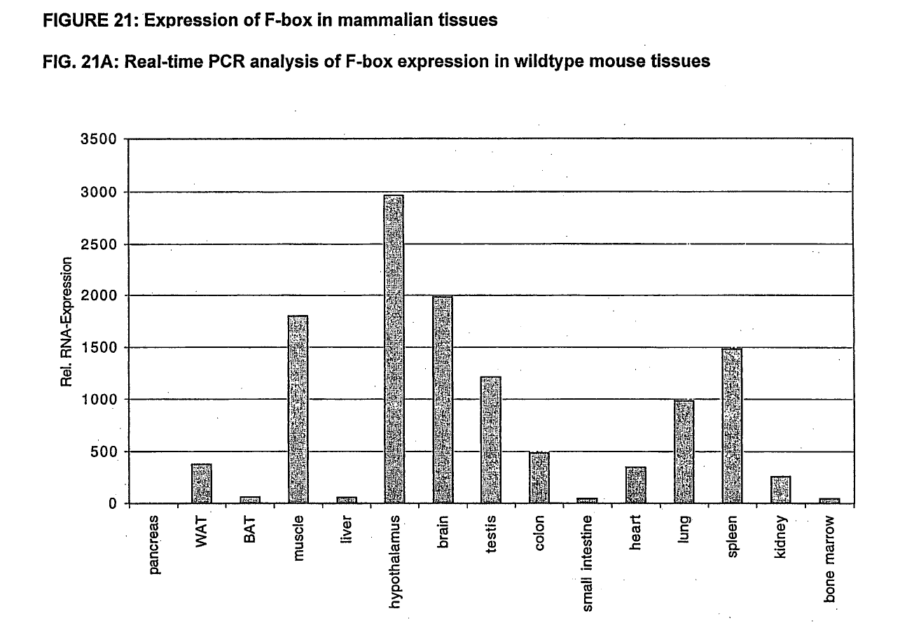

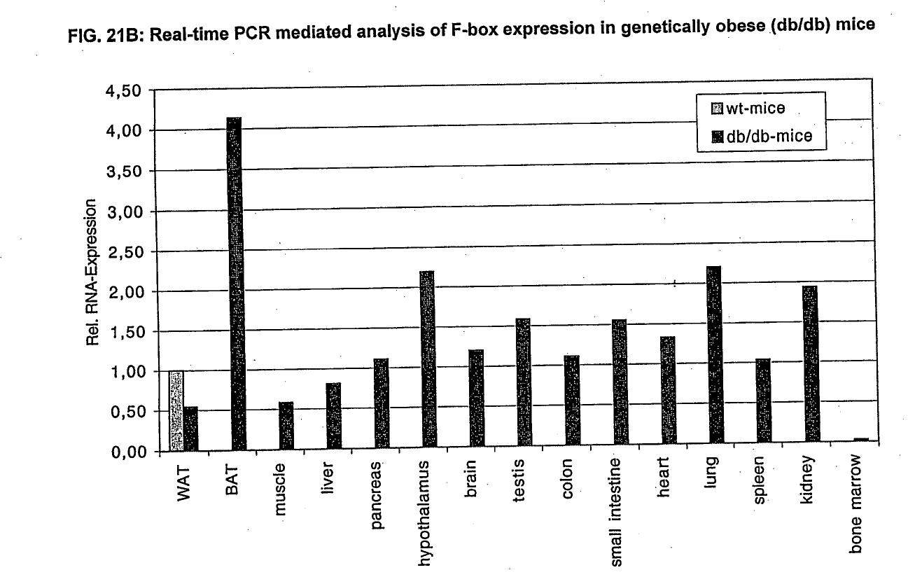

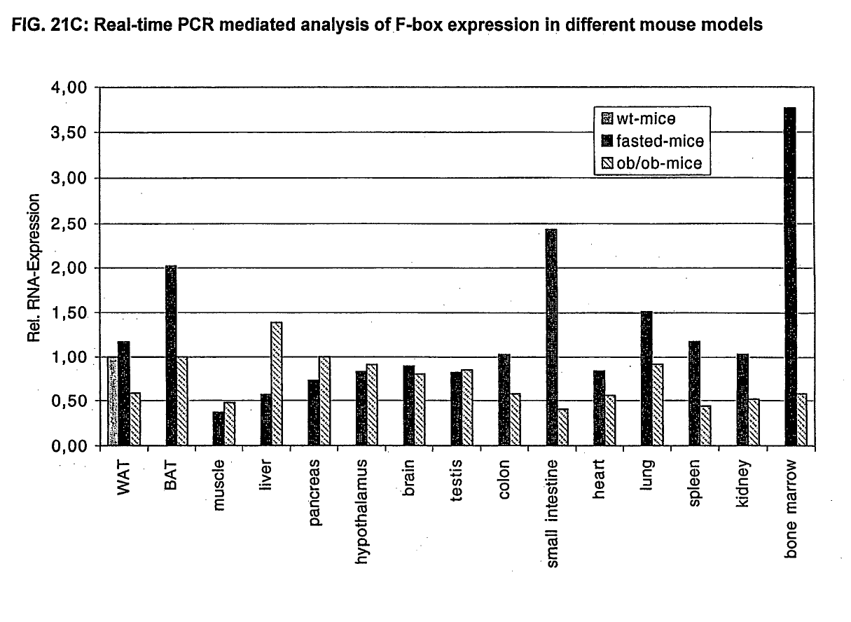

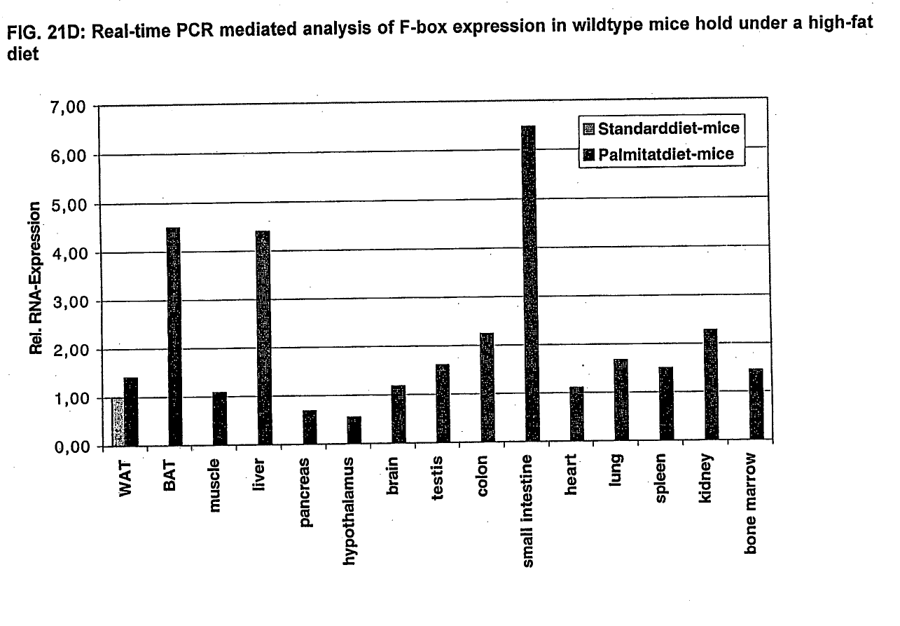

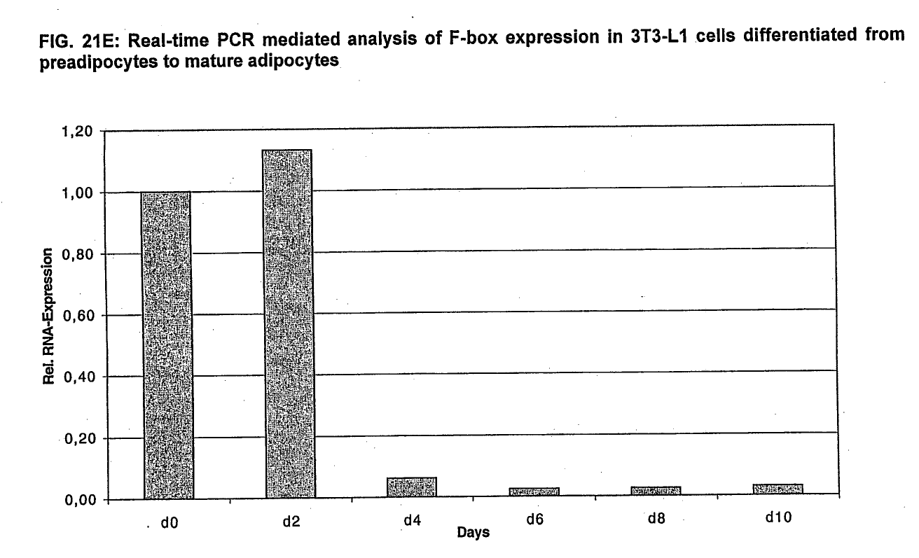

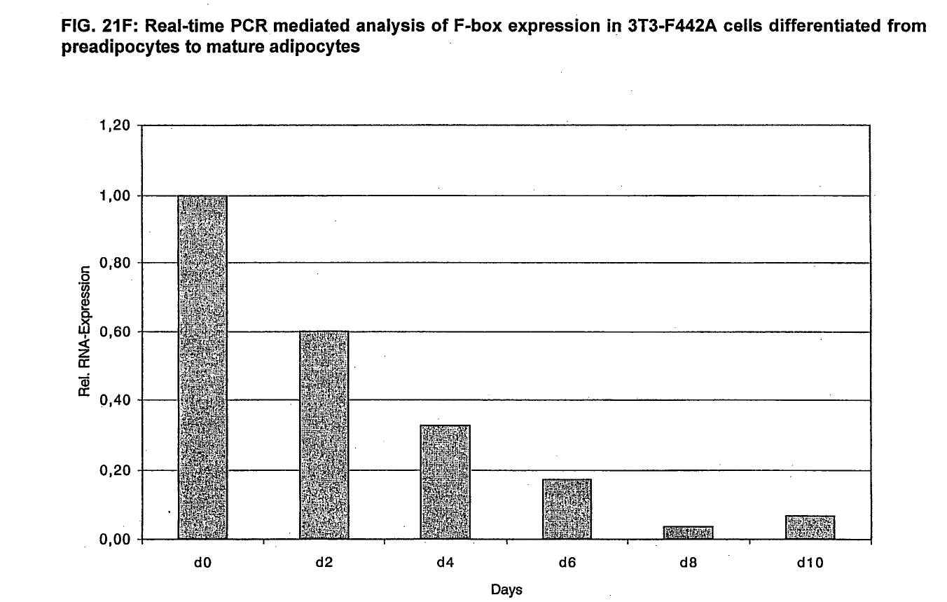

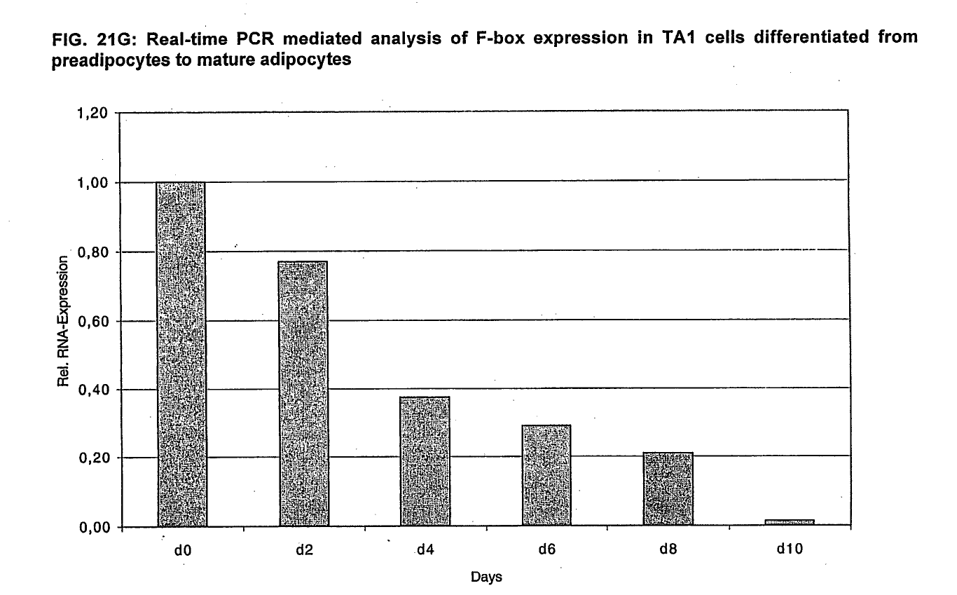

FIG. 21 shows the expression of the F-box genes in mammalian tissues.

FIG. 21A shows the real-time PCR analysis of F-box in wildtype mouse tissues.

FIG. 21B shows the real-time PCR mediated comparison of F-box expression in genetically obese (db/db) and wildtype mice.

FIG. 21C shows the real-time PCR mediated comparison of F-box expression in different mouse models.

FIG. 21D shows the real-time PCR mediated comparison of F-box expression in wildtype mice hold under a high fat diet.

FIG. 21E shows the real-time PCR mediated comparison of F-box expression during differentiation of mammalian fibroblast (3T3-L1) cells from pre-adipocytes to mature adipocytes.

FIG. 21F shows the real-time PCR mediated comparison of F-box expression during differentiation of mammalian fibroblast (3T3-F442A) cells from pre-adipocytes to mature adipocytes.

FIG. 21G shows the real-time PCR mediated comparison of F-box expression during differentiation of mammalian TA1 cells from pre-adipocytes to mature adipocytes.

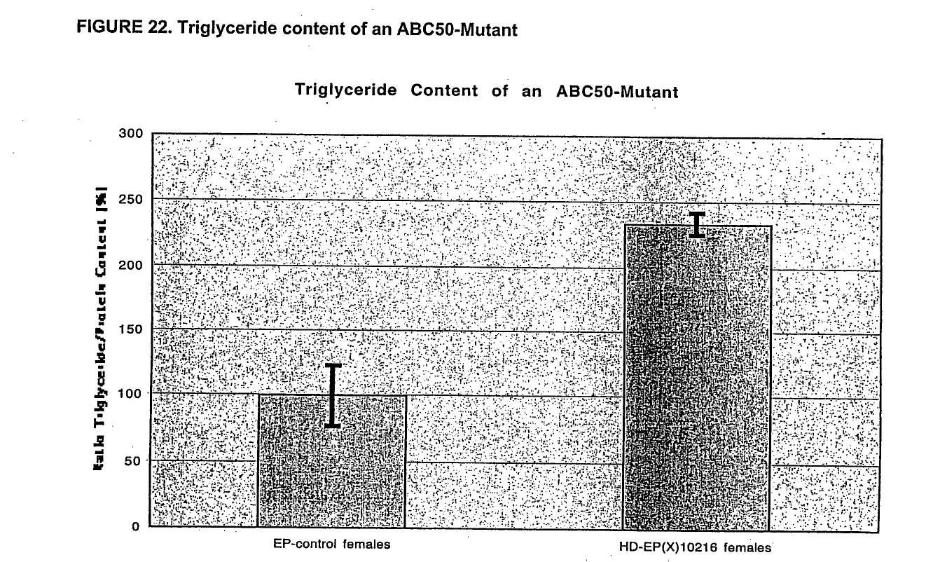

FIG. 22 shows the increase of triglyceride content of HD-EP(X)10216 ABC50 mutant flies caused by homozygous viable integration of the P-vector (in comparison to controls without integration of this vector).

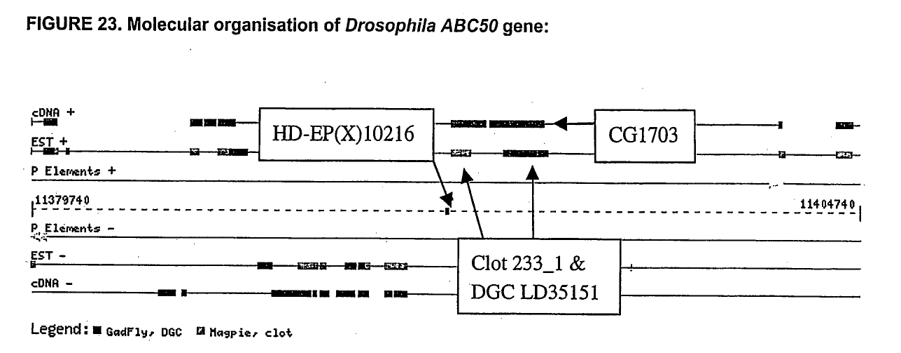

FIG. 23 shows the molecular organisation of the mutated ABC50 gene locus.

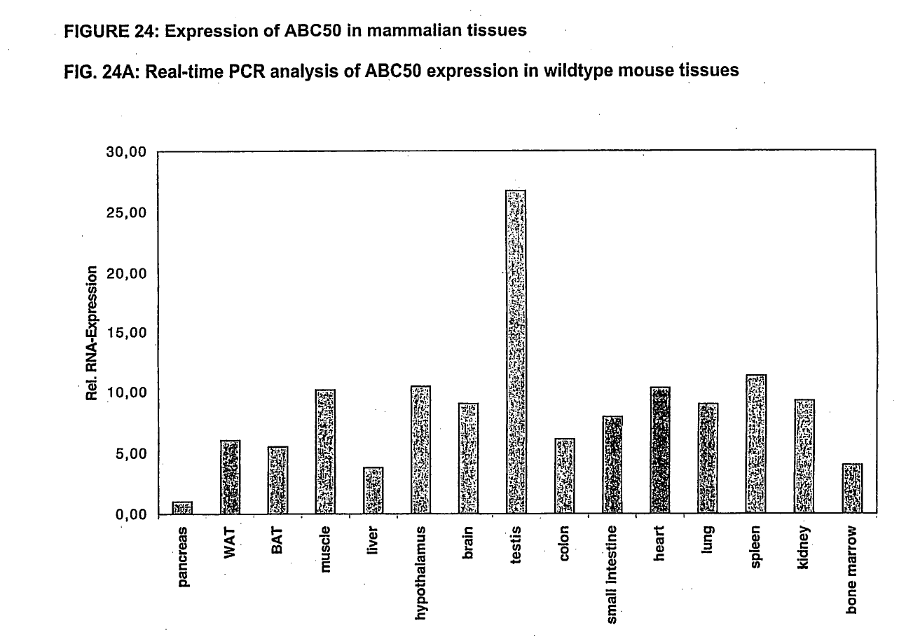

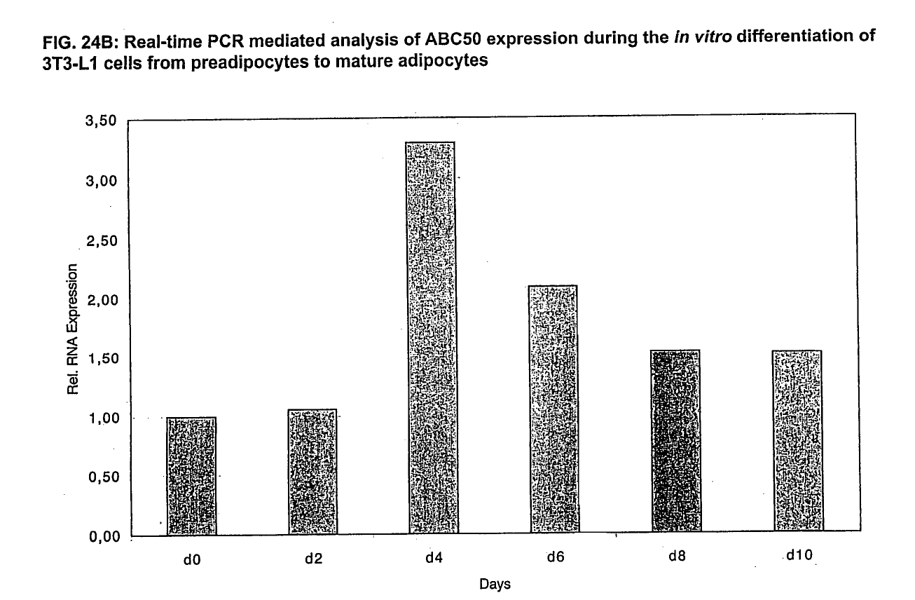

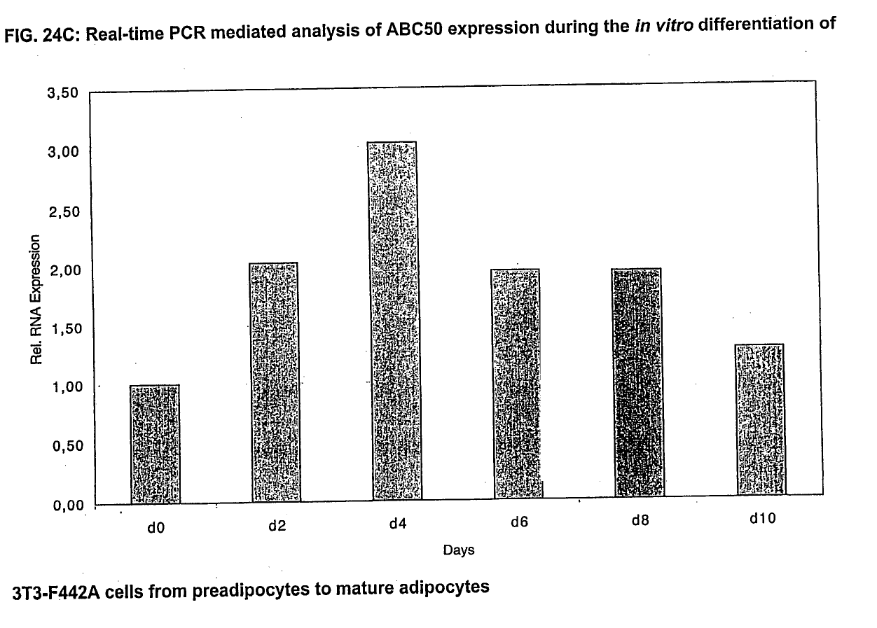

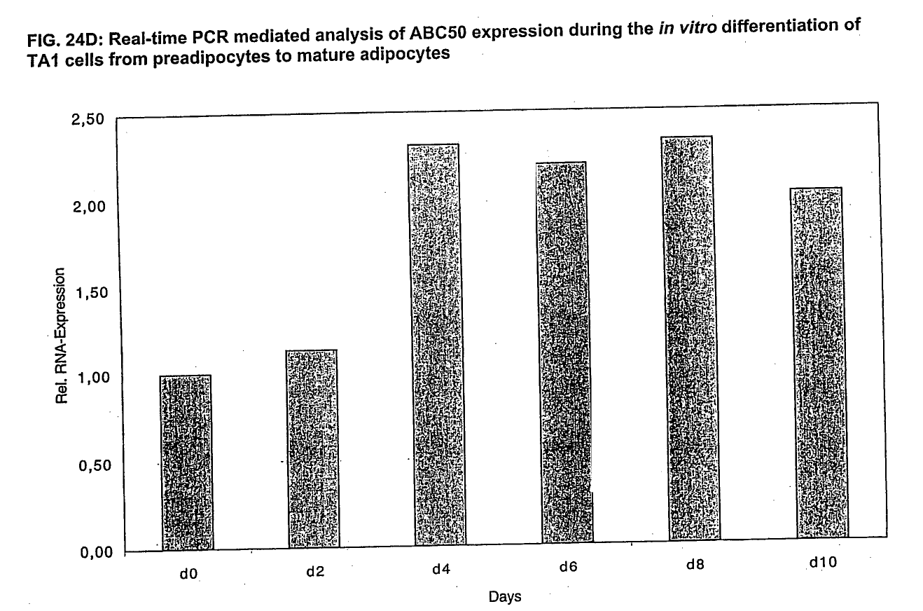

FIG. 24 shows the expression of the ABC50 gene in mammalian tissues.

FIG. 24A shows the real-time PCR analysis of ABC50 in wildtype mouse tissues.

FIG. 24B shows the real-time PCR mediated comparison of ABC50 expression during differentiation of mammalian fibroblast (3T3-L1) cells from pre-adipocytes to mature adipocytes.

FIG. 24C shows the real-time PCR mediated comparison of ABC50 expression during differentiation of mammalian fibroblast (3T3-F442A) cells from pre-adipocytes to mature adipocytes.

FIG. 24D shows the real-time PCR mediated comparison of ABC50 expression during differentiation of mammalian TA1 cells from pre-adipocytes to mature adipocytes.

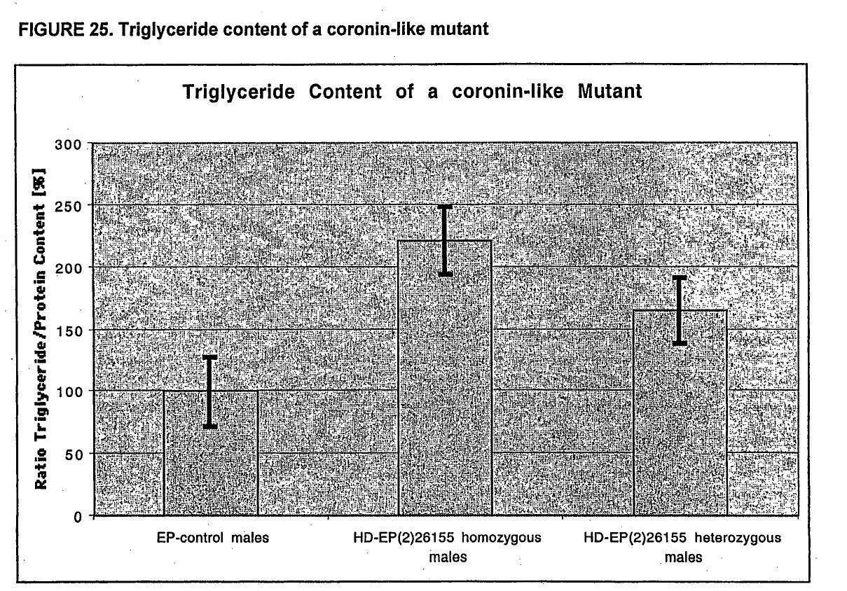

FIG. 25 shows the increase of triglyceride content of HD-EP(2)26155 coronin mutant flies caused by homozygous or heterozygous viable integration of the P-vector (in comparison to controls without integration of this vector).

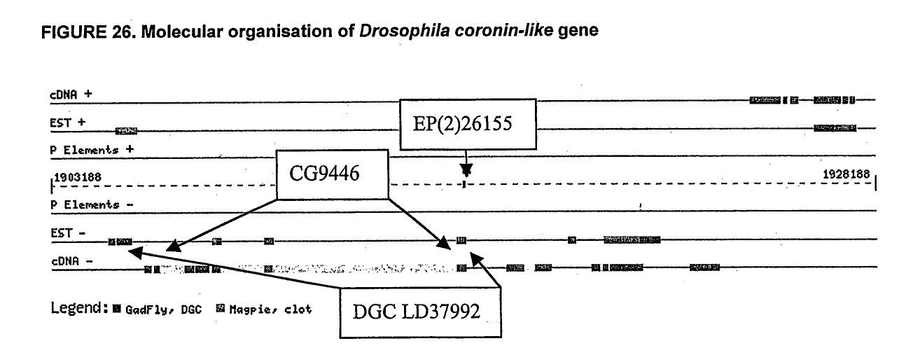

FIG. 26 shows the molecular organisation of the mutated coronin gene locus.

FIG. 27 shows the protein sequence (one-letter code) for human clipin E (SEQ ID NO: 8), which was reconstructed from human sequence NT010808 and mouse sequence BAB64362 using the program genewise

FIG. 28 shows the Clustal X (1.81) multiple sequence alignment of the amino acid sequences (one-letter code) for human coronin 1B (hs1B; GenBank Accession No. NP065174), human coronin 1C (hs1C; GenBank Accession No. NP055140), human clipinE (hs-clipin-gehewise; Seq ID NO: 8), and Drosophila coronin (CG9446; GadFly Accession Number CG9446). The identities are 53-54% and the similarities 68-70% between the human coronin proteins and the Drosophila protein.

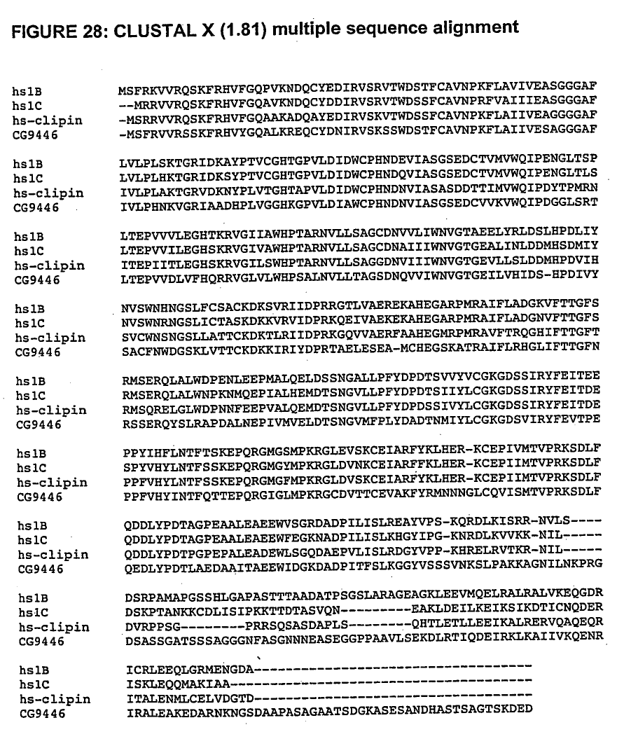

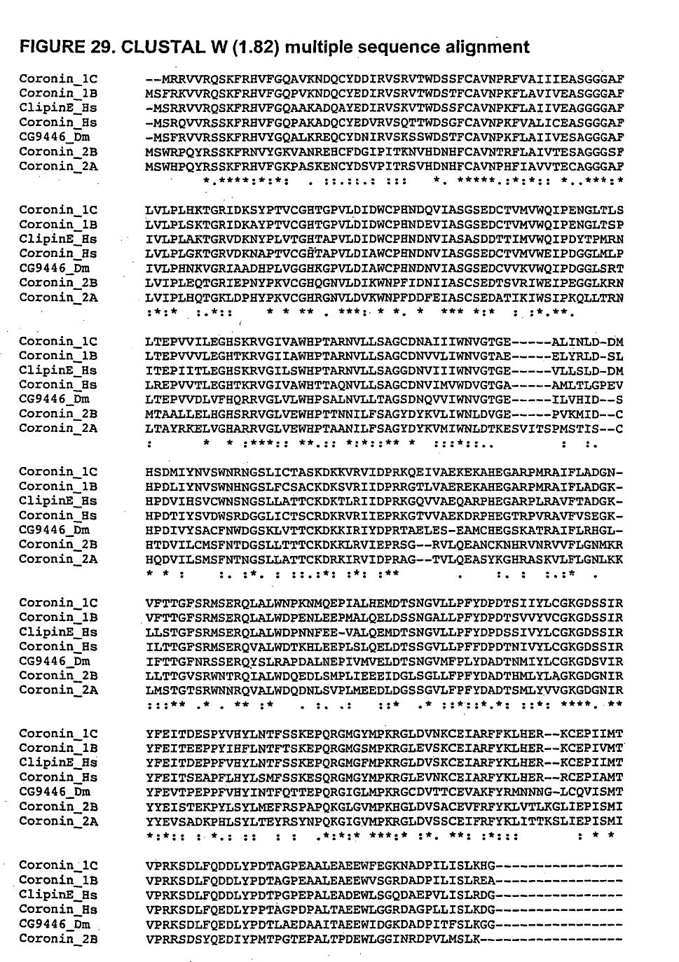

FIG. 29 shows the comparison (CLUSTAL W 1.82 multiple sequence alignment) of coronin proteins from different species, Coronin_{—}1C refers to human coronin 1C (GenBank Accession No. NP_{—}055140), Coronin_{—}1B refers to human coronin 1B (GenBank Accession No. NP_{—}065174), ClipinE_Hs refers to human clipin E (Seq ID NO: 8), Coronin_Hs refers to human coronin homologue (GenBank Accession No. CAA61482), CG9446_Dm refers to the protein encoded by Drosophila coro gene with GadFly Accession No. CG9446, Coronin_{—}2B refers to human Coronin 2B (GenBank Accession No. Q9UQ03), and Coronin_{—}2A refers to human Coronin 2A (GenBank Accession No. Q92828). Gaps in the alignment are represented as -.

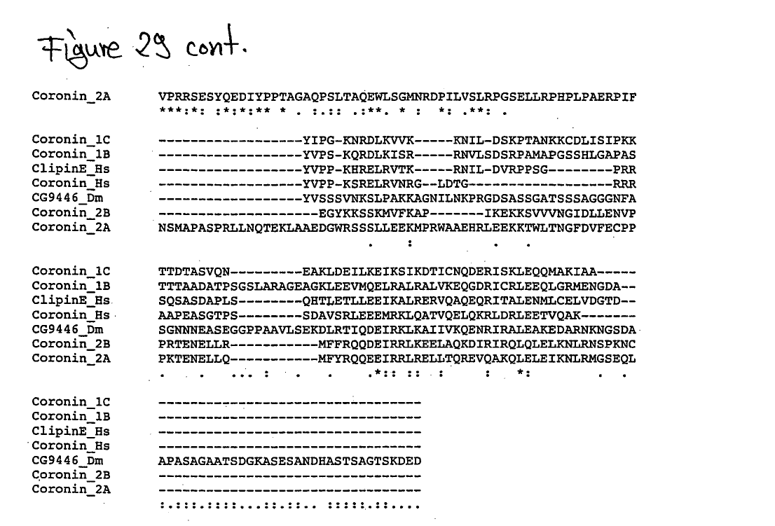

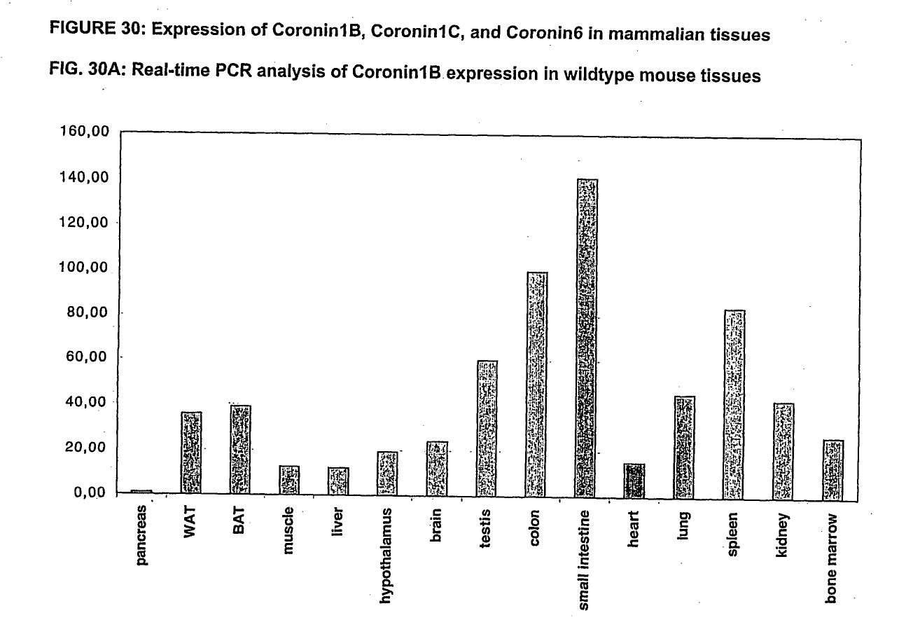

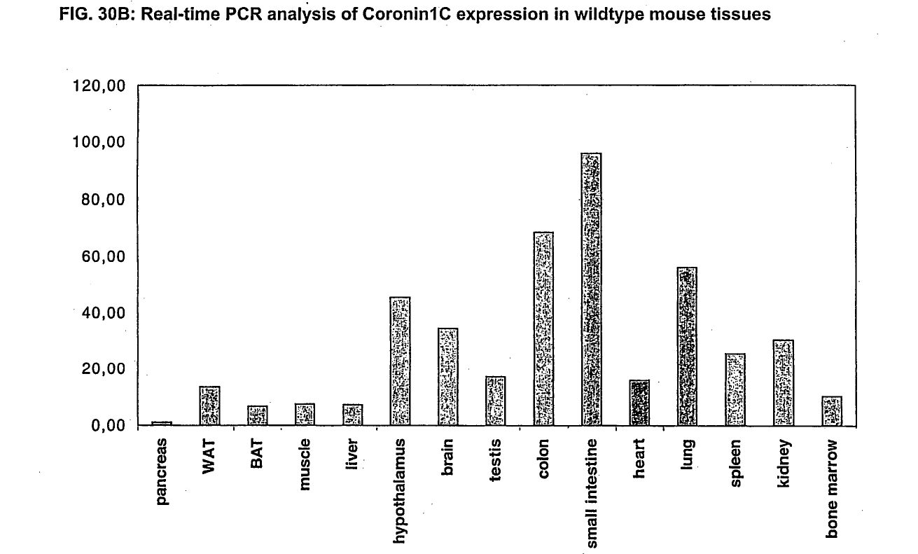

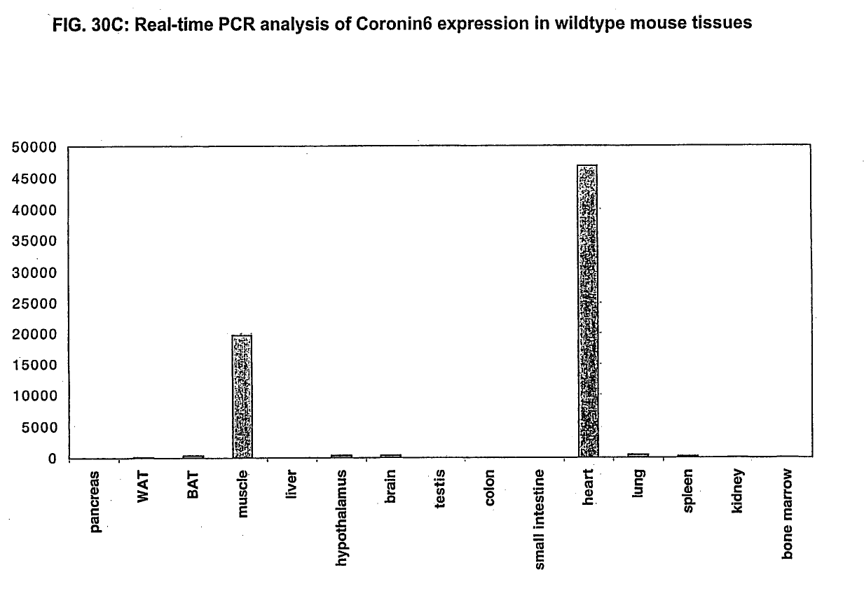

FIG. 30 shows the expression of the coronin 1B, Coronin1C, and Coronin6 genes in mammalian tissues.

FIG. 30A shows the real-time PCR analysis of Coronin1B in wildtype mouse tissues.

FIG. 30B shows the real-time PCR analysis of Coronin1C in wildtype mouse tissues.

FIG. 30C shows the real-time PCR analysis of Coronin6 in wildtype mouse tissues.

FIG. 30D shows the real-time PCR mediated comparison of Coronin 1C expression in different mouse models.

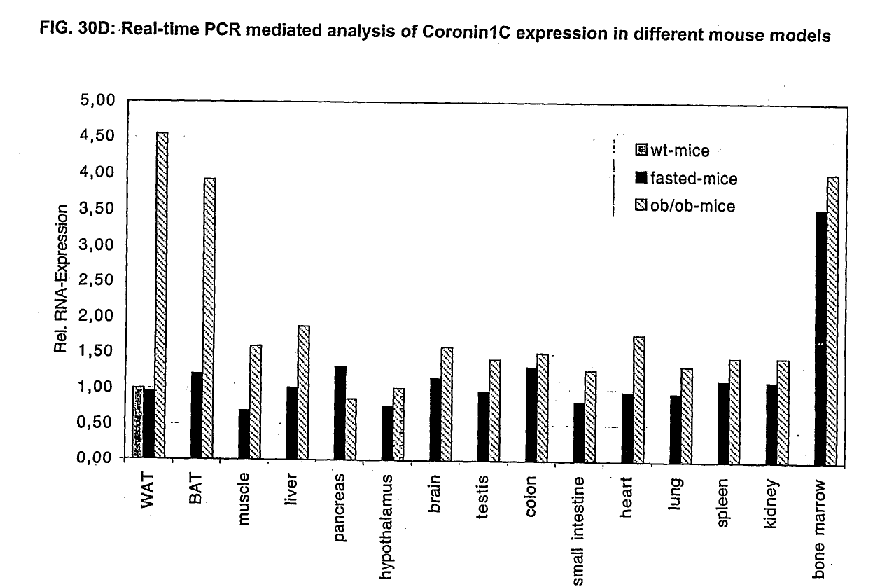

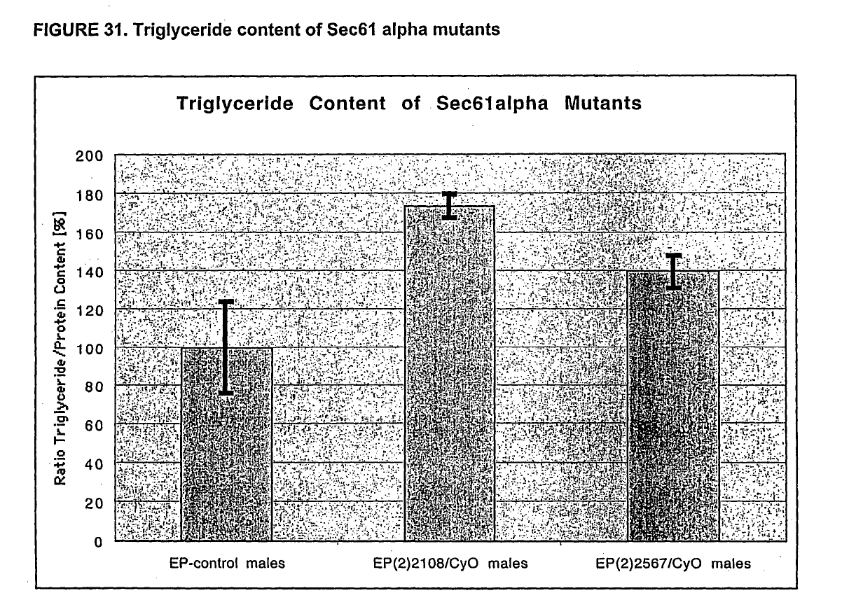

FIG. 31 shows the increase of triglyceride content of EP(2)2108 and EP(2)2567 Sec61 alpha mutant flies caused by heterozygous integration of the P-vector (in comparison to controls without integration of this vectors).

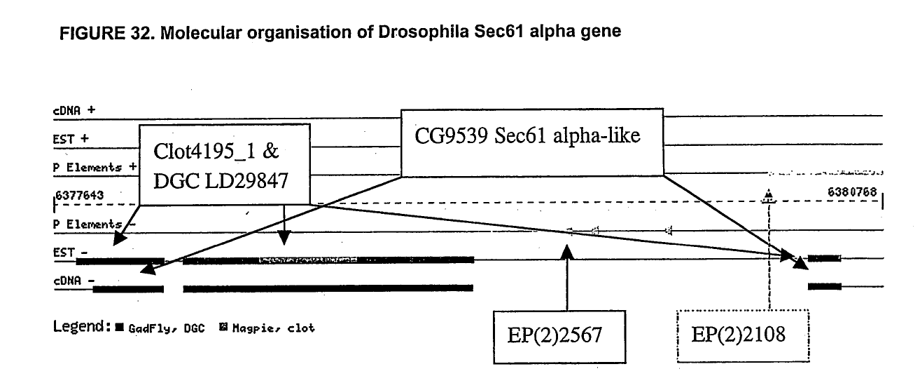

FIG. 32 shows the molecular organisation of the mutated Sec61 alpha gene locus.

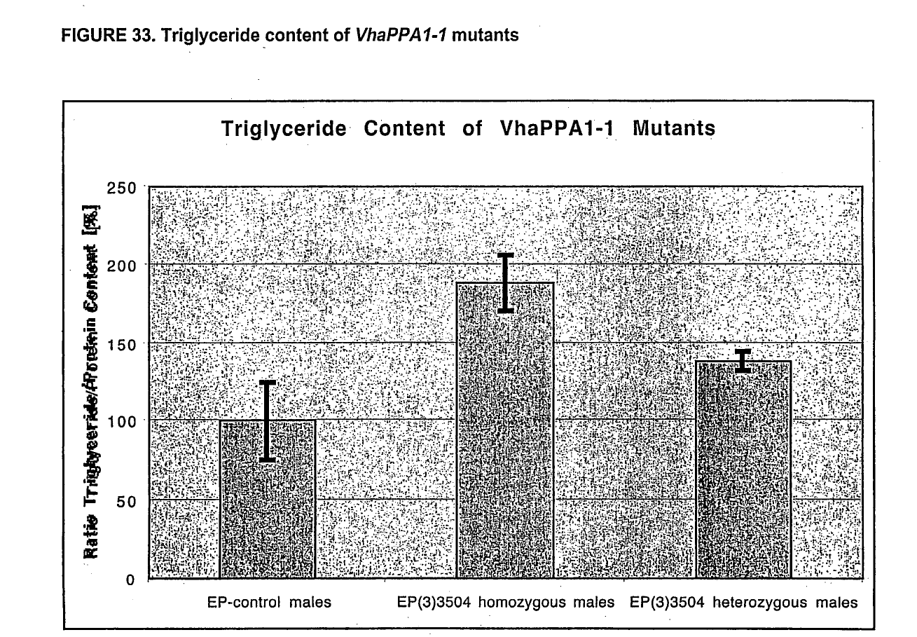

FIG. 33 shows the increase of triglyceride content of EP(3)3504 VhaPPA1-1 mutant flies caused by homozygous viable or heterozygous integration of the P-vector (in comparison to controls without integration of this vector).

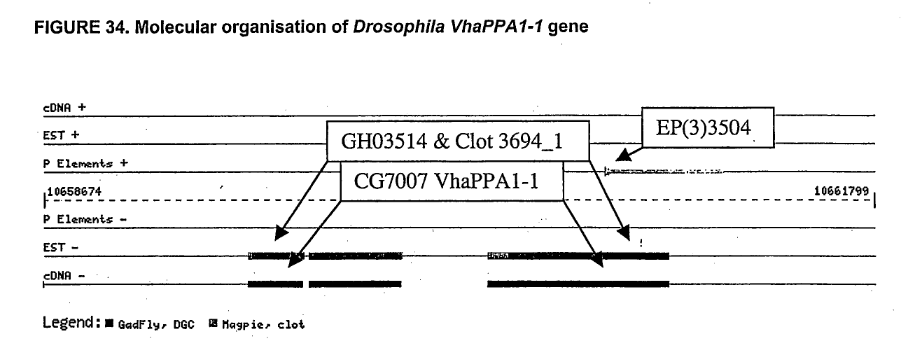

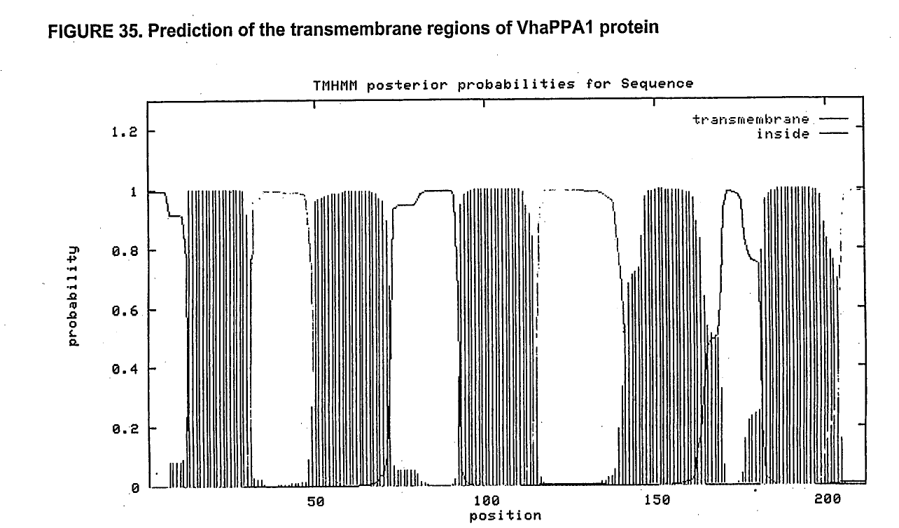

FIG. 34 shows the molecular organisation of the mutated VhaPPA1-1 gene locus.

FIG. 35 shows the transmembrane domain plot of VhaPPA1-1.

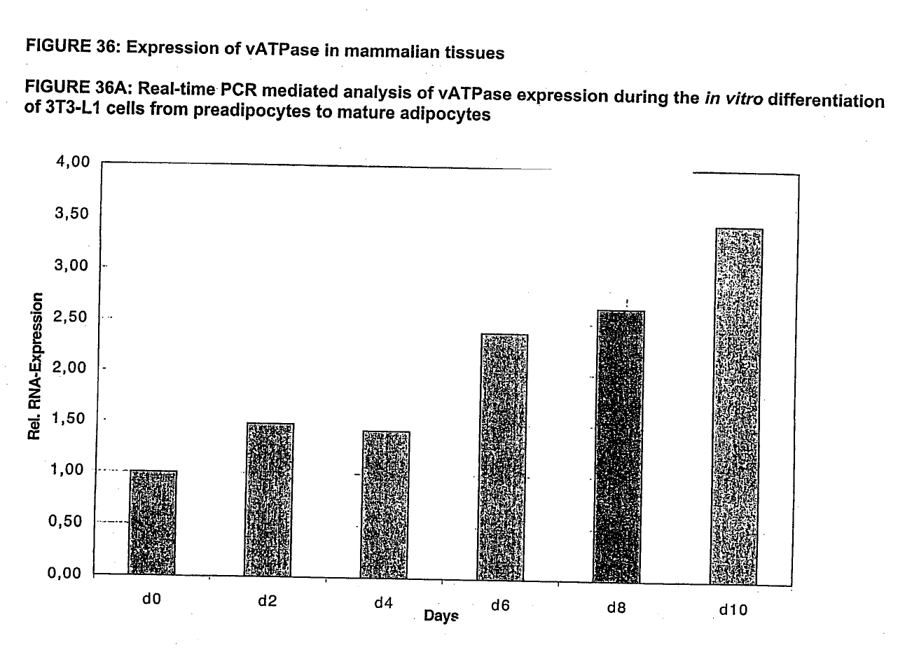

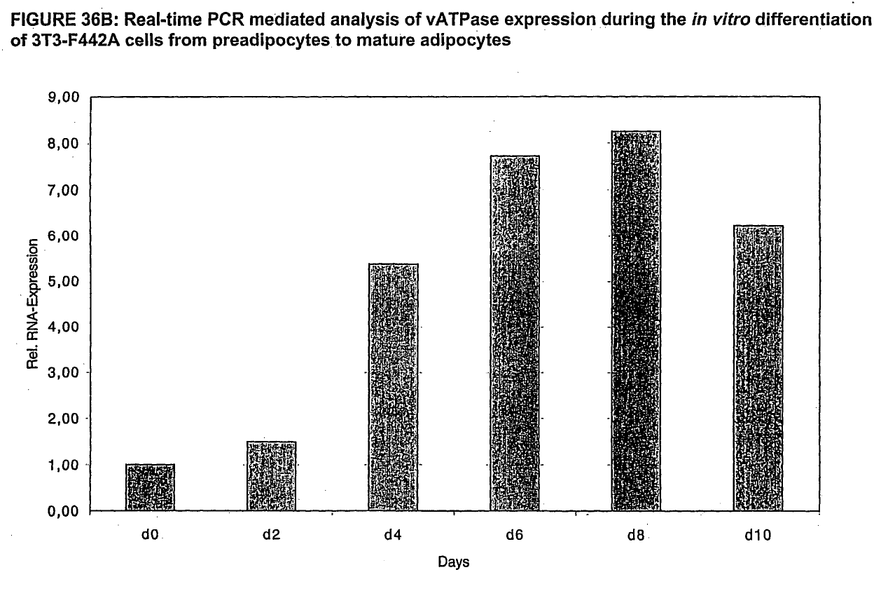

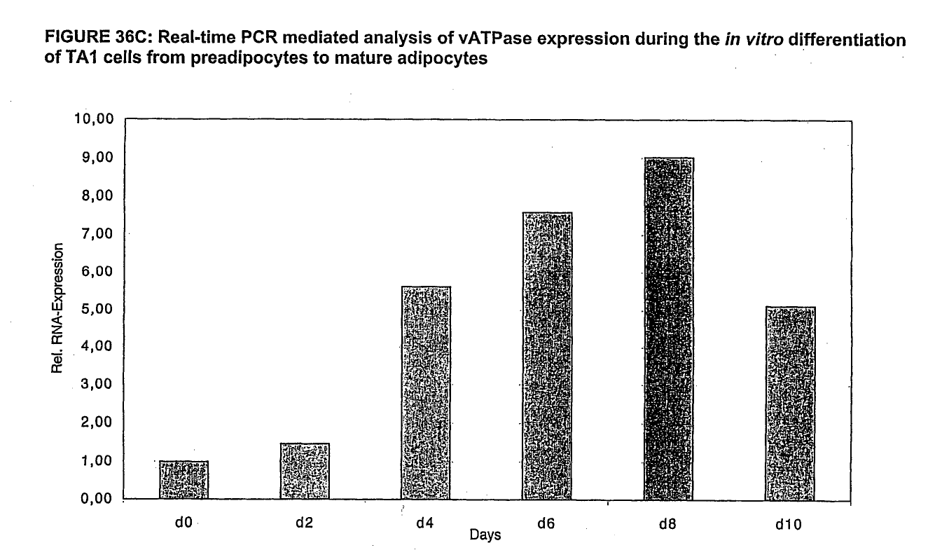

FIG. 36 shows the expression of the vATPase gene in mammalian tissues.

FIG. 36A shows the real-time PCR mediated comparison of vATPase expression during differentiation of mammalian fibroblast (3T3-L1) cells from pre-adipocytes to mature adipocytes.

FIG. 36B shows the real-time PCR mediated comparison of vATPase expression during differentiation of mammalian fibroblast (3T3-F442A) cells from pre-adipocytes to mature adipocytes.

FIG. 36C shows the real-time PCR mediated comparison of vATPase expression during differentiation of mammalian TA1 cells from pre-adipocytes to mature adipocytes.

The examples illustrate the invention:

### EXAMPLE 1

### Measurement of Triglyceride Content in Drosophila

The change of triglyceride content of Drosophila melanogaster containing a special expression system (EP-element; Rorth P., Proc Natl Acad Sci USA 1996, 93(22):12418-12422) was measured. Mutant flies are obtained from fly mutation stock collections (proprietary fly mutation stock collection; P Insertion Mutation Stock Center, Sezged, Hungary). The flies are grown under standard conditions known to those skilled in the art. In the course of the experiment, additional feedings with bakers yeast (Saccharomyces cerevisiae) are provided. The average increase of triglyceride content of Drosophila containing the HD-EP(3)31178, HD-EP(3)37100, EP(2)0641, HD-EP(2)26782, EP(3)3141, HD-EP(3) 31735, HD-EP(X)10216, HD-EP(2)26155, EP(2)2108, EP(2)2567, and EP(3)3504 vectors in homozygous or heterozygous integration was investigated in comparison to control flies (FIG. 1, 5, 8, 12, 17, 22, 25, 31, and 33). For determination of triglyceride, flies were incubated for 5 min at 90° C. in an aqueous buffer using a waterbath, followed by hot extraction. After another 5 min incubation at 90° C. and mild centrifugation, the triglyceride content of the flies extract was determined using Sigma Triglyceride (INT 336-10 or -20) assay by measuring changes in the optical density according to the manufacturer's protocol. As a reference protein content of the same extract was measured using BIO-RAD DC Protein Assay according to the manufacturer's protocol. The assays were repeated three times.

The average triglyceride level of all male flies of the EP collection (referred to as ‘EP-control males’) is shown as 100% in FIG. 1, 5, 8, 12, 17, 25, 31, and 33. The average triglyceride level of all female flies of the EP collection (referred to as ‘EP-control females’) is shown as 100% in FIG. 22.

Men

HD-EP(3)31178 and HD-EP(3)37100 homozygous flies show constantly a higher triglyceride content than the controls (approx. 35-60%; columns 2 and 3 in FIG. 1. Therefore, the loss of gene activity in the locus 87C9-87D1 on chromosome 3R where the EP-vector of HD-EP(3)31178 and HD-EP(3)37100 flies is homozygous viably integrated, is responsible for changes in the metabolism of the energy storage triglycerides, therefore representing in both cases a model for obese flies.

Gst2

EP(2)0641 homozygous flies (obtained from the P Insertion Mutation Stock Center, Sezged, Hungary) show constantly a higher triglyceride content than the controls (approx. 70%; column 2 in FIG. 5). Therefore, the loss of gene activity in the locus 53F11 on chromosome 2R where the EP-vector of EP(2)0641 flies is homozygous viably integrated, is responsible for changes in the metabolism of the energy storage triglycerides, therefore representing a model for obese flies. Even heterozygous integration of EP(2)0641 causes an increase of about 40% of the triglyceride content in flies (see column 3 in FIG. 5).

RabRP1

HD-EP(2)26782 homozygous flies show constantly a higher triglyceride content than the controls (approx. 75%; column 2 in FIG. 8). Therefore, the loss of gene activity in the locus 45B3-45B4 on chromosome 2R where the EP-vector of HD-EP(2)26782 flies is homozygous viably integrated, is responsible for changes in the metabolism of the energy storage triglycerides, therefore representing a model for obese flies. Even heterozygous integration of HD-EP(2)26782 causes an increase of about 60% of the triglyceride content in flies (see column 3 in FIG. 8).

Csp

EP(3)3141 homozygous flies (obtained from the P Insertion Mutation Stock Center, Sezged, Hungary) show constantly a higher triglyceride content than the controls (approx. 65%; column 2 in FIG. 12). Therefore, the loss of gene activity in the locus 79E1-2 on chromosome 3L where the EP-vector of EP(3)3141 flies is homozygous viably integrated, is responsible for changes in the metabolism of the energy storage triglycerides, therefore representing a model for obese flies.

F-Box

HD-EP(3)31735 homozygous flies show constantly a higher triglyceride content than the controls (approx. 140%; column 2 in FIG. 17). Therefore, the loss of gene activity in the locus 85C6-7 on chromosome 3R where the EP-vector of HD-EP(3)31735 flies is homozygous viably integrated, is responsible for changes in the metabolism of the energy storage triglycerides, therefore representing a model for obese flies.

ABC50

HD-EP(X)10216 homozygous flies show constantly a higher triglyceride content than the controls (approx. 130%; column 2 in FIG. 22). Therefore, the loss of gene activity in the locus 10C7 on chromosome X where the EP-vector of HD-EP(X)10216 flies is homozygous viably integrated, is responsible for changes in the metabolism of the energy storage triglycerides, therefore representing a model for obese flies.

Coronin

HD-EP(2)26155 homozygous flies show constantly a higher triglyceride content than the controls (approx. 125%; column 2 in FIG. 25), and even heterozygous flies show a higher triglyceride content than the controls (approx. 65%; column 3 in FIG. 25). Therefore, the loss of gene activity in the locus 42C8 on chromosome 2R where the EP-vector of HD-EP(2)26155 flies is homozygous viably or heterozygous integrated, is responsible for changes in the metabolism of the energy storage triglycerides, therefore representing a model for obese flies.

Sec61alpha

EP(2)2108 and EP(2)2567 heterozygous flies (obtained from the P Insertion Mutation Stock Center, Sezged, Hungary) show constantly a higher triglyceride content than the controls (approx. 75%; column 2 in FIG. 31 (‘EP(2)2108/CyO’); approx. 40% column 3 in FIG. 31(‘EP(2)2567/CyO’). Therefore, the loss of gene activity in the locus 26D6 on chromosome 2L where the EP-vector of EP(2)2108 and EP(2)2567 flies are heterozygous integrated, is responsible for changes in the metabolism of the energy storage triglycerides, therefore representing in both cases a model for obese flies.

VATPase

EP(3)3504 homozygous flies (obtained from the P Insertion Mutation Stock Center, Sezged, Hungary) show constantly a higher triglyceride content than the controls (approx. 185%; column 2 in FIG. 33). Therefore, the loss of gene activity in the locus 88D8 on chromosome 3R where the EP-vector of EP(3)3504 flies is homozygous viably integrated, is responsible for changes in the metabolism of the energy storage triglycerides, therefore representing a model for obese flies. Even heterozygous integration of EP(3)3504 causes an increase of about 40% of the triglyceride content in flies (see column 3 in FIG. 33).

### EXAMPLE 2

### Identification of Drosophila Genes Responsible for the Changes in the Metabolism of the Energy Storage Triglycerides

Using plasmid rescue method, genomic DNA sequences were isolated that are localized directly adjacent in 3prime direction of the integration site of the EP vectors (herein HD-EP(3)31178, HD-EP(3)37100, EP(2)0641, HD-EP(2)26782, EP(3)3141, HD-EP(3)31735, HD-EP(X)10216, HD-EP(2)26155, EP(2)2108, EP(2)2567, and EP(3)3504). Using those isolated genomic sequences, public DNA sequence databases like Berkeley Drosophila Genome Project (GadFly) were screened thereby identifying the integration sites of the vectors. FIGS. 2, 6, 9, 13, 18, 23, 26, 32, and 34 show the molecular organization of these gene loci.

Men

In FIG. 2, genomic DNA sequence is represented by the assembly as a dotted black line (from position 8468390 to 8480890 on chromosome 3R) that includes the integration sites of vector for line HD-EP(3)31178 and HD-EP(3)37100. Transcribed DNA sequences (expressed sequence tags, ESTs) and predicted exons are shown as bars in the lower two lines. Predicted exons of the cDNA with GadFly Accession Number CG10120 are shown as dark grey bars and introns as light grey bars. Men protein encodes for a gene that is predicted by GadFly sequence analysis programs as Accession Number CG10120. Public DNA sequence databases (for example, NCBI GenBank) were screened thereby identifying the integration sites of lines HD-EP(3)31178 and HD-EP(3)37100, causing an increase of triglyceride content. HD-EP(3)31178 and HD-EP(3)37100 is integrated into the second exon in sense orientation of the cDNA with GadFly Accession Number CG10120. Therefore, expression of the cDNA encoding Men (GadFly Accession Number CG10120) could be effected by homozygous integration of vectors of line HD-EP(3)31178 or HD-EP(3)37100, leading to increase of the energy storage triglycerides.

Gst2

In FIG. 6, genomic DNA sequence is represented by the assembly as a dotted black line (from position 1205500 to 12061250 on chromosome 2R) that includes the integration sites of vector for lines EP(2)0641. Transcribed DNA sequences (ESTs) and predicted exons are shown as bars in the lower two lines. Predicted exons of the cDNA with GadFly Accession Number CG8938 are shown as dark grey bars and introns as light grey bars. GST2 encodes for a gene that is predicted by GadFly sequence analysis programs as Accession Number CG8938. Public DNA sequence databases (for example, NCBI GenBank) were screened thereby identifying the integration site of line EP(2)0641, causing an increase of triglyceride content. EP(2)0641 is integrated 125 base pairs 5prime of the cDNA with Accession Number CG8938, encoding GST2 in sense orientation. Therefore, expression of the cDNA encoding Accession Number CG8938 could be effected by homozygous integration of vectors of line EP(2)0641, leading to increase of the energy storage triglycerides.

RabRP1

In FIG. 9, genomic DNA sequence is represented by the assembly as a dotted black line (from position 4210418 to 4235418 on chromosome 2R) that includes the integration site of vector for line HD-EP(2)26782. Transcribed DNA sequences (ESTs) and predicted exons are shown as bars in the lower two lines. Predicted exons of the cDNA with GadFly Accession Number CG8024 are shown as dark grey bars and introns as light grey bars. Rab-RP1 encodes for a gene that is predicted by GadFly sequence analysis programs as Accession Number CG8024. Public DNA sequence databases (for example, NCBI GenBank) were screened thereby identifying the integration site of line HD-EP(2)26782, causing an increase of triglyceride content. HD-EP(2)26782 is integrated in the cDNA at approximately 20 base pairs in antisense orientation of GadFly Accession Number CG8024, encoding Rab-RP1. Therefore, expression of the cDNA encoding GadFly Accession Number CG8024 could be effected by homozygous or heterozygous integration of vectors of line HD-EP(2)26782, leading to increase of the energy storage triglycerides.

Csp

In FIG. 13, genomic DNA sequence is represented by the assembly as a dotted black line (from position 22101652 to 22114152 on chromosome 3L) that includes the integration site of vector for line EP(3)3141. Transcribed DNA sequences (ESTs) and predicted exons are shown as bars in the upper two lines. Predicted exons of the cDNA with GadFly Accession Number CG6395 are shown as dark grey bars and introns as light grey bars. Csp encodes for a gene that is predicted by GadFly sequence analysis programs as Accession Number CG6395. Public DNA sequence databases (for example, NCBI GenBank) were screened thereby identifying the integration site of line EP(3)3141, causing an increase of triglyceride content. EP(3)3141 is integrated into the cDNA at the second intron in antisense orientation of Accession Number CG6395, encoding cysteine string protein Csp. Therefore, expression of the cDNA encoding Accession Number CG6395 could be effected by homozygous integration of vectors of lines EP(3)3141, leading to increase of the energy storage triglycerides.

F-box

In FIG. 18, genomic DNA sequence is represented by the assembly as a dotted black line (from position 4858500 to 4871000 on chromosome 3R) that includes the integration site of vector for line HD-EP(3)31735. Transcribed DNA sequences (ESTs) and predicted exons are shown as bars in the upper two lines. Predicted exons of the cDNA with GadFly Accession Number CG11033 are shown as dark grey bars and introns as light grey bars. F-box protein Lilina/FBL7 encodes for a gene that is predicted by GadFly sequence analysis programs as Accession Number CG11033. Public DNA sequence databases (for example, NCBI GenBank) were screened thereby identifying the integration site of line HD-EP(3)31735, causing an increase of triglyceride content. HD-EP(3)31735 is integrated into the promoter of in the 5prime in antisense orientation of the cDNA with Accession Number CG11033. Therefore, expression of the cDNA encoding Accession Number CG11033 could be effected by homozygous integration of vectors of line HD-EP(3)31735, leading to increase of the energy storage triglycerides.

ABC50

In FIG. 23, genomic DNA sequence is represented by the assembly as a dotted black line (from position 11379740 to 11404740 on chromosome X) that includes the integration site of vector for line HD-EP(X)10216. Transcribed DNA sequences (ESTs) and predicted exons are shown as bars in the upper two lines. Predicted exons of the cDNA with GadFly Accession Number CG1703 are shown as dark grey bars and introns as light grey bars. ABC50 encodes for a gene that is predicted by GadFly sequence analysis programs as Accession Number CG1703. Public DNA sequence databases (for example, NCBI GenBank) were screened thereby identifying the integration sites of lines HD-EP(X)10216, causing an increase of triglyceride content. HD-EP(X)10216 is integrated 45 base pairs 5prime in antisense orientation of the cDNA with Accession Number CG1703. Therefore, expression of the cDNA encoding Accession Number CG1703 could be effected by homozygous integration of vectors of line HD-EP(X) 10216, leading to increase of the energy storage triglycerides.

Coronin

In FIG. 26, genomic DNA sequence is represented by the assembly as a dotted black line (from position 1903188 to 1928188 on chromosome 2R) that includes the integration site of vector for line HD-EP(2)26155. Transcribed DNA sequences (ESTs) and predicted exons are shown as bars in the lower two lines. Predicted exons of the cDNA with GadFly Accession Number CG9446 are shown as dark grey bars and introns as light grey bars coronin encodes for a gene that is predicted by GadFly sequence analysis programs as Accession Number CG9446. Public DNA sequence databases (for example, NCBI GenBank) were screened thereby identifying the integration sites of lines HD-EP(2)26155, causing an increase of triglyceride content. HD-EP(2)26155 is integrated at about base pair 50 of the cDNA with Accession Number CG9446, encoding coronin in sense orientation. Therefore, expression of the cDNA encoding Accession Number CG9446 could be effected by homozygous integration of vectors of lines HD-EP(2)26155, leading to increase of the energy storage triglycerides.

Sec61alpha

In FIG. 32, genomic DNA sequence is represented by the assembly as a dotted black line (from position 6377343 to 6380768 on chromosome 2L) that includes the integration sites of vector for lines EP(2)2108 and EP(2)2567. Transcribed DNA sequences (ESTs) and predicted exons are shown as bars in the lower two lines. Predicted exons of the cDNA with GadFly Accession Number CG9539 are shown as dark grey bars and introns as light grey bars. Sec61 alpha encodes for a gene that is predicted by GadFly sequence analysis programs as Accession Number CG9539. Public DNA sequence databases (for example, NCBI GenBank) were screened thereby identifying the integration sites of lines EP(2)2108 and EP(2)2567, causing an increase of triglyceride content. EP(2)2108 and EP(2)2567 are both integrated in the first intron of the cDNA with Accession Number CG9539, encoding Sec61 alpha. Therefore, expression of the cDNA encoding Accession Number CG9539 could be effected by heterozygous integration of vectors of lines EP(2)2108 and EP(2)2567, leading to increase of the energy storage triglycerides.

vATPase

In FIG. 34, genomic DNA sequence is represented by the assembly as a dotted black line (from position 10658674 to 10661799 on chromosome 3R) that includes the integration site of vector for line EP(3)3504. Transcribed DNA sequences (ESTs) and predicted exons are shown as bars in the lower two lines. Predicted exons of the cDNA with GadFly Accession Number CG7007 are shown as dark grey bars and introns as light grey bars. VhaPPA1-1 encodes for a gene that is predicted by GadFly sequence analysis programs as Accession Number CG7007. Public DNA sequence databases (for example, NCBI GenBank) were screened thereby identifying the integration site of line EP(3)3504, causing an increase of triglyceride content. EP(3)3504 is integrated into the first exon in antisense orientation of the cDNA with Accession Number CG7007. Therefore, expression of the cDNA encoding Accession Number CG7007 could be effected by homozygous integration of vectors of line EP(3)3504, leading to increase of the energy storage triglycerides.

### EXAMPLE 3

### Identification of Mammalian Men, GST2, Rab-RP1, Csp, F-Box Lilina/FBL7, ABC50, Coronin, Sec61 Alpha, or VhaPPA1-1 Protein and Gene Homologs

The proteins of the invention and homologous proteins and nucleic acid molecules coding therefore are obtainable from insect or vertebrate species, e.g. mammals or birds. Particularly preferred are nucleic acids encoding the Drosophila or human homologs of the proteins of the invention. Sequences homologous to Drosophila proteins of the invention were identified using the publicly available program BLASTP 2.2.3 of the non-redundant protein data base of the National Center for Biotechnology Information (NCBI) (see, Altschul et al., 1997, Nucleic Acids Res. 25:3389-3402).

Men

Drosophila Men protein is in 545 amino acids 58% identical and 73% similar to human cytosolic malic enzyme 1 (ME1), NADP(+)-dependent (GenBank Accession Number NM_{—}002395 for the cDNA, NP_{—}002386 for the protein), localized on chromosome 6. Drosophila Men protein is over 537 amino acids 56% identical and 73% similar to human mitochondrial malic enzyme 3, NADP(+)-dependent, pyruvic-malic carboxylase, malate dehydrogenasae, NADP-ME (GenBank Accession Number NM_{—}006680 for the cDNA, NP_{—}006671 for the protein), localized on chromosome 11. Drosophila Men protein is also homologous to human mitochondrial malic enzyme 2, NAD(+)-dependent (GenBank Accession No. NM_{—}002396.2 for the cDNA, NP_{—}002387 for the protein). An alignment of MEN from different species has been done by the ClustaW program (see also FIG. 3A).

Gst2

Particularly preferred are human GST2 homologous nucleic acids, particularly nucleic acids encoding a human hematopoietic prostaglandin D2 synthase (glutathione-requiring prostaglandin D synthase, PGDS; GenBank Accession No. NM_{—}014485 for the cDNA, NP_{—}055300 for the protein), mouse hematopoietic prostaglandin D2 synthase 2 (Ptgds2; GenBank Accession No. NM_{—}019455 for the cDNA), and rat hematopoietic prostaglandin D2 synthase 2 (Ptgds2; GenBank Accession No. NM_{—}31644 for the cDNA). An alignment of GST2 from different species has been done by the ClustaW program.

RabRP

Particularly preferred are human Rab-RP1 homologous nucleic acids, particularly nucleic acids encoding a human Rab32 (GenBank Accession No. NM_{—}006834 for the cDNA, NP_{—}006825 for the protein, formerly XM_{—}004076, human Rab38 (GenBank Accession No. NM_{—}022337 for the cDNA, NP_{—}071732 for the protein, formerly XM_{—}015771, and human Rab7 (GenBank Accession No. NM_{—}003929 for the cDNA, NP_{—}003920 for the protein). Drosophila Gene CG8024 shows 67% identity and 78% similiarity to human Rab32 in 209 amino acids, and Drosophila Gene CG8024 shows 72% identity and 83% similiarity to human Rab38 in 176 amino acids. An alignment of Rab-RP1 from Drosophila and human has been done by the ClustaW program (see also FIG. 10). NEU Drosophila RabRP1 also shows homology to mouse Rab32 (GenBank Accession No. NM_{—}026405 for the cDNA) and mouse Rab38 (GenBank Accession No. NM_{—}028238 for the cDNA).

Csp

Particularly preferred are human Csp homologous nucleic acids, particularly nucleic acids encoding human Csp (EnsEMBL accession number ENST00000217123 for the cDNA; GenBank Accession Number CAC15495.1 for the protein; see FIG. 14, SEQ ID NO: 7), human cysteine string protein 1 (GenBank Accession No. S70515 for the protein), human gamma cysteine string protein (unnamed protein product; GenBank Accession No. AK097736 for the cDNA, BAC05155 for the protein), human Beta cysteine string protein (GenBank Accession No. Q9UF47). The cDNA shown in FIG. 14 was generated from the genomic sequence AL118506 (located on human chromosom 20) by applying the Genscan program. Drosophila Gene CG6395 shows 61% identity and 73% similiarity to human cysteine string protein (Accession Number CAC15495.1) in 165 amino acids (amino acids 8 to 165 in CG6395). The highest similiarity is found in the conserved DNAJ—domain and the cys-string of these proteins, with 77% identity and 88% similiarity in the DNAJ domain. An alignment of Csp from different species has been done by the ClustaW program.

F-Box

Particularly preferred are nucleic acids encoding Drosophila F-box protein Lilina/FBL7 (GadFly Accession Number CG11033), human F-box protein Lilina/FBL7 (similar to human F-box and leucine-rich repeat protein 11; GenBank Accession No. NM_{—}012308 for the cDNA and NP_{—}036440.1 for the protein), human JEMMA protein (GenBank Accession No. CAD30700 for the protein), NEU PDH finger protein 2 (GenBank Accession Number NM_{—}005392 for the cDNA, NP_{—}005383 for the protein), NEU human protein similar to several hypothetical proteins (GenBank Accession No. AAC83407 for the protein). An alignment of F-box protein Lilina/FBL7 from different species has been done by the ClustaL X (1.8.) multiple sequence alignment program (see FIG. 19) and Clustal W (1.82) multiple sequence alignment program (see FIG. 20).

ABC50

Particularly preferred are human ABC50 homologous nucleic acids, particularly nucleic acids encoding a human ABC50 protein (TNF-alpha stimulated ABC protein; GenBank Accession No. AF027302 for the cDNA, AAC70891 for the protein) and homologous genes of Drosophila ABC50 (GadFly Accession Number CG1703), and rat ABC50 (GenBank Accession No. AF293383 for the cDNA). An alignment of ABC50 from different species has been done by the ClustaW program.

Coronin

Particularly preferred are human coronin homologous nucleic acids, particularly nucleic acids encoding human actin-binding protein coronin 1B (GenBank Accession No. NM_{—}020441 for the cDNA, NP_{—}065174 for the protein, formerly GenBank Accession No. BC006449), human actin-binding protein coronin 1C (GenBank Accession No. NM_{—}014325 for the cDNA, NP_{—}055140 for the protein; GenBank Accession No. BC002342), human coronin protein (coronin homologue; GenBank Accession No. X89109 for the cDNA, CAA61482 for the protein), human clipinE/coronin 6type B (see FIG. 27; Seq ID NO: 8), human Coronin 2A (GenBank Accession No. Q92828 for the protein), and human Coronin 2B (GenBank Accession No. Q9UQ03 for the protein). An alignment of coronin from different species has been done by the ClustaW program (see also FIG. 29).

Sec61alpha

Particularly preferred are human Sec61 alpha homologous nucleic acids, particularly nucleic acids encoding a human Sec61 alpha form 2 protein (GenBank Accession No. NM_{—}018144 for the cDNA, NP_{—}060614 for the protein, formerly GenBank Accession No. AF346603) and human Sec61 alpha form 1 protein (GenBank Accession No. NM_{—}013336.2 for the cDNA, NP_{—}037468 for the protein, formerly AF346602). An alignment of Sec61 alpha from different species has been done by the ClustaW program. Drosophila Sec61 alpha is also homologous to mouse Sec61 alpha-2 protein (GenBank Accession No. AF222748) and mouse Sec61 isoform 1 protein (GenBank Accession No. AF145253).

vATPase

Particularly preferred are nucleic acids encoding a Drosophila VhaPPA1-1 (GadFly Accession Number CG7007), human ATPase, H+transporting, lysosomal 21 kD (vacuolar protein pump) protein (GenBank Accession No. NM_{—}004047 for the cDNA, NP_{—}004038 for the protein), and mouse ATPase, H+transporting, lysosomal 21 kD (vacuolar protein pump) protein (GenBank Accession No. NM_{—}033617 for the cDNA). An alignment of VhaPPA1-1 from different species has been done by the ClustaW program. A comparison between the Drosophila and the human vacuolar ATPase (GenBank Accession No. NP_{—}004038) shows 63% identity (124 of 194 amino acids) and 76% similarity (150 of 194 amino acids).

### EXAMPLE 4

### Expression of the Polypeptides in Mammalian (Mouse) Tissues

For analyzing the expression of the polypeptides disclosed in this invention in mammalian tissues, several mouse strains (preferrably mice strains C57BI/6J, C57BI/6 ob/ob and C57BI/KS db/db, which are standard model systems in obesity and diabetes research) were purchased from Harlan Winkelmann (33178 Borchen, Germany) and maintained under constant temperature (preferrably 22° C.), 40% humidity and a light/dark cycle of preferrably 14/10 hours. The mice were fed a standard chow (for example, from ssniff Spezialitaten GmbH, order number ssniff M-Z V1126-000). For the fasting experiment (“fasted wild type mice”), wild type mice were starved for 48 h without food, but only water supplied ad libitum. (see, for example, Schnetzler et al. J Clin Invest 1993 July;92(1):272-80, Mizuno et al. Proc Natl Acad Sci U S A 1996 April 16;93(8):3434-8). Animals were sacrificed at an age of 6 to 8 weeks. Male mice (preferrably mouse strain C57BL/6) were placed at the age of 4 weeks in groups of 8 animals (N=8) for 16 weeks on control diet (preferably Altromin C1057 mod control, 4.5% crude fat, or high fat diet (preferably Altromin C1057 mod. high fat, 23.5% crude fat). At the age of 20 weeks mice were sacrified and different tissues and organs dissected. The animal tissues and organs were isolated according to standard procedures known to those skilled in the art, snap frozen in liquid nitrogen and stored at −80° C. until needed.

For analyzing the role of the proteins disclosed in this invention in the in vitro differentiation of different mammalian cell culture cells for the conversion of pre-adipocytes to adipocytes, mammalian fibroblast (3T3-L1) cells (e.g., Green & Kehinde, Cell 1: 113-116, 1974) were obtained from the American Tissue Culture Collection (ATCC, Hanassas, Va., USA; ATCC-CL 173). 3T3-L1 cells were maintained as fibroblasts and differentiated into adipocytes as described in the prior art (e.g., Qiu. et al., J. Biol. Chem. 276:11988-95, 2001; Slieker et al., BBRC 251: 225-9, 1998). At various time points of the differentiation procedure, beginning with day 0 (day of confluence) and day 2 (hormone addition; for example, dexamethason and 3-isobutyl-1-methylxanthin), up to 10 days of differentiation, suitable aliquots of cells were taken every two days. Alternatively, mammalian fibroblast 3T3-F442A cells (e.g., Green H. and Kehinde O., (1976) Cell 7(1): 105-113) were obtained from the Harvard Medical School, Department of Cell Biology (Boston, Mass., USA). Alternatively, mammalian fibroblast TA1 cells (Chapman A. B. et al., (1984) J Biol Chem 259(24):15548-15555) were obtained from ATCC. 3T3-F442A and TA1 cells were maintained as fibroblasts and differentiated into adipocytes as described previously (Djian, P. et al., (1985) J. Cell. Physiol. 124 (3):554-556). At various time points of the differentiation procedure, beginning with day 0 (day of confluence and hormone addition, for example, Insulin), up to 10 days of differentiation, suitable aliquots of cells were taken every two days. 3T3-F442A cells and TA1cells are differentiating in vitro already in the confluent stage after hormone (insulin) addition.

RNA was isolated from mouse tissues or cell culture cells using Trizol Reagent (for example, from Invitrogen, Karlsruhe, Germany) and further purified with the RNeasy Kit (for example, from Qiagen, Germany) in combination with an DNase-treatment according to the instructions of the manufacturers and as known to those skilled in the art. Total RNA was reverse transcribed (preferrably using Superscript II RNaseH-Reverse Transcriptase, from Invitrogen, Karlsruhe, Germany) and subjected to Taqman analysis preferrably using the Taqman 2xPCR Master Mix (from Applied Biosystems, Weiterstadt, Germany; the Mix contains according to the Manufacturer for example AmpliTaq Gold DNA Polymerase, AmpErase UNG, dNTPs with dUTP, passive reference Rox and optimized buffer components) on a GeneAmp 5700 Sequence Detection System (from Applied Biosystems, Weiterstadt, Germany).

For the analysis of the expression of the proteins of the invention, taqman analysis was performed using the following primer/probe pairs:
Mouse Men forward primer (Seq ID NO: 9) 5′-GGG AGA CCT TGG CTG TAA TGG-3′;
Mouse Men reverse primer (Seq ID NO: 10) 5′-ACC CCT CCA CAT GCC GT-3′;
Mouse Men Taqman probe (Seq ID NO: 11) (5/6-FAM) CAT CCC TGT GGG TAA ACT GGC CCT TT (5/6-TAMRA)
Mouse m2GST2 forward primer (Seq ID NO: 12) 5′-CAA GCC AAC TCT TCC ATT TGG-3′;
Mouse m2GST2 reverse primer (Seq ID NO: 13) 5′-ATT GCG AGG CTC TGG TGG-3′;
Mouse m2GST2 Taqman probe (Seq ID NO: 14) (5/6-FAM) ATC CCT GTT TTG GAG GTG GAA GGA CTT ACA (5/6-TAMRA)
Mouse Rab32 forward primer (Seq ID NO: 15) 5′-GGT CCC AGT GCT GCT GAT GT-3′;
Mouse Rab32 reverse primer (Seq ID NO: 16) 5′-CCT CCA TAC AGG CAG GAC CA-3′;
Mouse Rab32 Taqman probe (Seq ID NO: 17) (5/6-FAM) TCT CTG TGC CCC ATG TGC TGT CTC C (5/6-TAMRA)
Mouse Rab38 forward primer (Seq ID NO: 18) 5′-ACC TCA CAA GGA GCA CCT GTA CA-3′;
Mouse Rab38 reverse primer (Seq ID NO: 19) 5′-TAA TGC TGG TCT TGC CCA CA-3′;
Mouse Rab38 Taqman probe (Seq ID NO: 20) (5/6-FAM) TGC TGG TGA TCG GCG ACC TGG (5/6-TAMRA)
Mouse Csp forward primer (Seq ID NO: 21) 5′-GGC ACA GCT GCA GTC TGA TG-3′;
Mouse Csp reverse primer (Seq ID NO: 22) 5′-TGG CAG ATG CTG GCT GTA TG-3′;
Mouse Csp Taqman probe (Seq ID NO: 23) (5/6-FAM) AAG GGA GGC TAC AGA CAC ACC GAT CG (5/6-TAMRA)
Mouse F-box forward primer (Seq ID NO: 24) 5′-CGT CGC CAG ACC CTG ATT-3′;
Mouse F-box reverse primer (Seq ID NO: 25) 5′-CAA ACG GCG GCT CCC-3′;
Mouse F-box Taqman probe (Seq ID NO: 26) (5/6-FAM) CAC AGT CCG AGA CGT CAA ACT CCT GGT (5/6-TAMRA)
Mouse ABC50 forward primer (Seq ID NO: 27) 5′-TCG ACA TGG ACT CCC GGA T-3′;
Mouse ABC50 reverse primer (Seq ID NO: 28) 5′-CAG GAG TAG TGT GCT CTT CCC C-3′;
Mouse ABC50 Taqman probe (Seq ID NO: 29) (5/6-FAM) TGC ATC GTG GGT CCC AAT GGT G (516-TAMRA)
Mouse Coronin 1B forward primer (Seq ID NO: 30) 5′-AGG GAC CAT CTC CTC GAC CT-3′;
Mouse Coronin 1B reverse primer (Seq ID NO: 31) 5′-CCC ATC TCT GCT GCT TTT TCT G-3′;
Mouse Coronin 1B Taqman probe (Seq ID NO: 32) (5/6-FAM) CCC AAC CCA CTG CCC CCT CA (5/6-TAMRA)
Mouse Coronin 1C forward primer (Seq ID NO: 33) 5′-CCG CGC ACT CCCAGG-3′;
Mouse Coronin 1C reverse primer (Seq ID NO: 34) 5′-CAA ATC TGA CAT GGA ATG TCT CCA-3′;
Mouse Coronin 1C Taqman probe (Seq ID NO: 35) (5/6-FAM) AGG GCA GAG AGG GAG ACA CTG CCA (5/6-TAMRA)
Mouse Coronin 6 forward primer (Seq ID NO: 36) 5′-TGA GAC CCA TGC GGG CT-3′;
Mouse Coronin 6 reverse primer (Seq ID NO: 37) 5′-TCG GGT GAA TCC CGT GG-3′;
Mouse Coronin 6 Taqman probe (Seq ID NO: 38) (516-FAM) TCT TCA CGC GGC TGG GTC ATA TCT TC (5/6-TAMRA)
Mouse vATPaseVO forward primer (Seq ID NO: 39) 5′-GGC TTG GTG TTC AGG GTC TC-3′;
Mouse vATPaseVO reverse primer (Seq ID NO: 40) 5′-ACT GCA ATG CCT CCA GAG TCA-3′;
Mouse vATPaseVO Taqman probe (Seq ID NO: 41) (5/6-FAM) CCT GCA CTC ACC TCT TGC TGC CTG (5/6-TAMRA)

The results of the real time PCR (Taqman) analysis are shown in the FIGS. 4, 7, 11, 16, 21, 24, 30, and 36.

Men

As shown in FIG. 4A, real time PCR (Taqman) analysis of the expression of the Men protein in mammalian (mouse) tissues revealed that Men is expressed in different mammalian tissues, showing higher levels of expression in BAT, testis, kidney, liver, and WAT tissues. With regard to changes in expression intensity during the differentiation of preadipocytes to adipocytes, a strong increase in relative signal intensity can be observed for Men during the in vitro differentiation program of 3T3-L1 cells (see

FIG. 4B). The results of both experiments show that Men plays a role in the metabolism of adipose tissue and therefore suggests a relevance of this gene for metabolic disorders.

Gst2

The expression of the m2GST2 is strongly upregulated in WAT of both animal models of obesity used in these experiments. In two more tissues which are highly relevant for metabolic disorders, namely BAT and muscle the expression of m2GST2 is also upregulated in both models. These expression patterns strongly suggest that m2GST2 has an essential function in adipose tissue and muscle. In contrast m2GST-2 was not expressed in the 3T3-L1 adipocte cell line (Data not shown). As m2GST-2 is most likely involved in the synthesis of signalling molecules it is likely that its expression is under the control of external stimuli, which are not present in the cell culture system used. An indication for this is the strong response observed in our animal models.

Rab32 and Rab38

Taqman analysis revealed that Rab32 and Rab38 are equally interesting homologues of the fly gene. Both are rather ubiquitously expressed with Rab38 showing a stronger expression in lung, spleen and kidney (FIG. 11B). Both genes show an upregulation of their expression in WAT and BAT of genetically ob/ob mice (FIGS. 11C and 11D). A further example of the regulation of these genes under different metabolic conditions is provided by their marked downregulation in WAT, BAT and muscle of fasted mice. In addition, a significant upregulation in kidney of fasted mice is noted (FIGS. 11C and 11D). The upregulation of Rab32 and Rab38 is also observed in WAT, BAT and heart of the genetically obese db/db mice (FIGS. 11E and 11F). Expression of Rab32 is induced during the in vitro differentiation of 3T3-L1 cells from preadipocytes to adipocytes (FIG. 11G).

Csp

Taqman analysis revealed that CSP is consistently upregulated during the in vitro differentiation of preadipocytes to adipocytes (FIGS. 16A, 16B and 16C).

F-Box

Taqman analysis revealed that F-Box is rather ubiquitously expressed (FIG. 21A). F-Box expression is under metabolic control: In fasted as to well as obese (db/db) mice, expression is increased in brown adipose tissue (FIGS. 21B and 21C). In addition, expression of F-Box is strongly induced in BAT, liver and small intestine in mice hold under a high fat diet (FIG. 21D). During the in vitro differentiation of 3T3-L1 as well as of two additional model systems for the in vitro differentiation of preadipocytes to adipocytes, the 3T3-F442A and TA1, cell lines, the expression of F-Box is dramatically reduced (FIGS. 21E, 21F, and 21G).

ABC50

As shown in FIG. 24A, real time PCR (Taqman) analysis of the expression of the ABC50 protein in mouse tissues revealed that ABC50 is expressed in different mammalian tissues, showing higher levels of expression in testis, spleen, heart, hypothalamus, and muscle tissues. With regard to changes in expression intensity during the differentiation of preadipocytes to adipocytes, Taqman analysis revealed a consistently upregulated expression of ABC50 during the in vitro differentiation of preadipocytes to adipocytes (see FIGS. 24B, 24C, and 24D).

Coronin

Taqman analysis revealed that Coronin 1C is the more interesting homologue of the fly gene. In comparison to Coronin 6, which is restricted to muscle and heart, Coronin 1B and 1C are ubiquitously expressed with clear expression in WAT and BAT (FIGS. 30A, 30B, and 30C). The expression of Coronin 1C in white and brown adipose tissue is under metabolic control: in genetically obese (ob/ob) mice, expression of Coronin 1C is strongely induced in these tissues compared to wildtype levels (FIG. 30D).

vATPase

Taqman analysis revealed that vATPaseVO shows a clear upregulation of its expression intensity during the differentiation of preadipocytes to adipocytes (FIGS. 36A, 36B, and 36C).

All publications and patents mentioned in the above specification are herein incorporated by reference.

Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A pharmaceutical composition comprising a nucleic acid molecule of the Men (malic enzyme) gene family, the GST2 (glutathione S-transferase 2) gene family, the Rab-RP1 family of proteins, the cystein string protein (Csp) family of proteins, the F-box gene family, the ABC50 gene family, the coronin family of actin-associated proteins, the Sec61 alpha gene family, or the vacuolar ATPase gene family or a polypeptide encoded thereby or a fragment or a variant of said nucleic acid molecule or said polypeptide or an antibody, an aptamer or another receptor recognizing a nucleic acid molecule of the Men, GST2, Rab-RP1, Csp, F-box protein Lilina/FBL7, ABC50, coronin, Sec61 alpha, or VhaPPA1-1 gene family or a polypeptide encoded thereby together with pharmaceutically acceptable carriers, diluents and/or adjuvants.

2. The composition of claim 1, wherein the nucleic acid molecule is a vertebrate or insect Men protein, GST2, Rab-RP1, Csp, F-box protein Lilina/FBL7, ABC50, coronin, Sec61 alpha, or VhaPPA1-1 nucleic acid, particularly encoding human Men proteins (malic enzyme 1; GenBank Accession No. NM_{—}002395 for the cDNA, NP_{—}002386 for the protein, malic enzyme 3; NM_{—}006680 for the cDNA, NP_{—}006671 for the protein, malic enzyme 2; GenBank Accession No. NM_{—}002396.2 for the cDNA, NP_{—}002387 for the protein), a human GST2 protein (hematopoietic prostaglandin D2 synthase; GenBank Accession No. NM_{—}014485 for the cDNA, NP_{—}055300 for the protein), a mouse GST2 protein (hematopoietic prostaglandin D2 synthase 2; GenBank Accession No. NM_{—}019455 for the cDNA, NP_{—}062328 for the protein), a rat GST2 protein (hematopoietic prostaglandin D2 synthase 2; GenBank Accession No. NM_{—}031644 for the cDNA, NP_{—}113832 for the protein), human Rab-RP1 proteins (Rab32; GenBank Accession No. NM_{—}006834 for the cDNA, NP_{—}006825 for the protein, formerly GenBank Accession No. XM_{—}004076, Rab38; GenBank Accession No. NM_{—}022337 for the cDNA, NP_{—}071732 for the protein, formerly XM_{—}015771, Rab7; GenBank Accession No. NM_{—}003929 for the cDNA, NP_{—}003920 for the protein), mouse Rab-RP1 proteins (Rab32; GenBank Accession No. NM_{—}026405 for the cDNA, NP_{—}080681 for the protein, Rab38; GenBank Accession No. NM_{—}028238 for the cDNA, NP_{—}082514 for the protein), human Csp proteins (Csp; EnsEMBL Accession No. ENST00000217123 (SEQ ID NO. 7) for the cDNA, GenBank Accession No. CAC15495.1 for the protein, Csp1; GenBank Accession No. S70515 for the protein, gamma Csp; GenBank Accession No. AK097736 for the cDNA, BAC05155 for the protein, Beta Csp; GenBank Accession No. Q9UF47 for the protein), human F-box Lilina/FBL7 proteins (F-box and leucine-rich repeat protein 11; GenBank Accession No. NM_{—}012308 for the cDNA, NP_{—}036440 for the protein, JEMMA protein; GenBank Accession No. CAD30700 for the protein, PDH finger protein 2; GenBank Accession Number NM_{—}005392 for the cDNA, NP_{—}005383 for the protein, protein similar to several hypothetical proteins; GenBank Accession No. AAC83407 for the protein), a human ABC50 protein (TNF-alpha stimulated ABC protein; GenBank Accession No. AF027302 for the cDNA, AAC70891 for the protein), a rat ABC50 protein (GenBank Accession No. AF293383 for the cDNA, AAG23960 for the protein), human coronin proteins (actin-binding protein coronin 1B; GenBank Accession No. NM_{—}020441 for the cDNA, NP_{—}065174 for the protein, formerly BC006449, actin-binding protein coronin 1C; GenBank Accession No. NM_{—}014325 for the cDNA, NP_{—}055140 for the protein; formerly BC002342, coronin homologue; GenBank Accession No. X89109 for the cDNA, CAA61482 for the protein, clipinE/coronin 6 type B; Seq ID NO: 8, Coronin 2A; GenBank Accession No. Q92828 for the protein, Coronin 2B; GenBank Accession No. Q9UQ03 for the protein), human Sec61 alpha proteins (Sec61 alpha form 2 protein; GenBank Accession No. NM_{—}018144 for the cDNA, NP_{—}060614 for the protein, formerly AF346603, human Sec61 alpha form 1 protein; GenBank Accession No. NM_{—}013336.2 for the cDNA, NP_{—}037468 for the protein, formerly AF346602), mouse Sec61 alpha proteins (Sec61 alpha-2 protein; GenBank Accession No. AF222748 for the cDNA, AAG44253 for the protein, Sec61 alpha isoform 1 protein; GenBank Accession No. AF145253 for the cDNA. AAF66695 for the protein) or a human VhaPPA1-1 protein (ATPase, H+transporting, lysosomal 21 kD (vacuolar protein pump) protein; GenBank Accession No. NM_{—}004047 for the cDNA, NP_{—}004038 for the protein), or mouse VhaPPA1-1 protein (ATPase, H+transporting, lysosomal 21 kD protein; GenBank Accession No. NM_{—}033617 for the cDNA, NP_{—}291095 for the protein), or a Drosophila Men protein (GadFly Accession Number CG10120), a Drosophila GST2 protein (GstS1; GadFly Accession Number CG8938), a Drosophila Rab-RP1 protein (GadFly Accession Number CG8024), a Drosophila Csp protein (GadFly Accession Number CG6395), a Drosophila F-box protein Lilina/FBL7 (GadFly Accession Number CG11033), a Drosophila ABC50 protein (GadFly Accession Number CG1703), a Drosophila coro protein (GadFly Accession Number CG9446), a Drosophila sec61 alpha protein (GadFly Accession Number CG9539), or a Drosophila VhaPPA1-1 protein (GadFly Accession Number CG7007), or a fragment thereof or a variant thereof and/or a nucleic acid molecule complementary thereto.

3. The composition of claim 1, wherein said nucleic acid molecule
(a) hybridizes at 50□C in a solution containing 1×SSC and 0.1% SDS to a nucleic acid molecule as defined in claim 2 and/or a nucleic acid molecule which is complementary thereto;
(b) it is degenerate with respect to the nucleic acid molecule of (a)
(c) encodes a polypeptide which is at least 85%, preferably at least 90%, more preferably at least 95%, more preferably at least 98% and up to 99.6% identical to the polypeptides as defined in claim 2;
(d) differs from the nucleic acid molecule of (a) to (c) by mutation and wherein said mutation causes an alteration, deletion, duplication or premature stop in the encoded polypeptide.

4. The composition of claim 1, wherein the nucleic acid molecule is a DNA molecule, particularly a cDNA or a genomic DNA.

5. The composition of claim 1, wherein said nucleic acid encodes a polypeptide contributing to regulating the energy homeostasis and/or the metabolism of triglycerides.

6. The composition of claim 1, wherein said nucleic acid molecule is a recombinant nucleic acid molecule.

7. The composition of any claim 1, wherein the nucleic acid molecule is a vector, particularly an expression vector.

8. The composition of claim 1, wherein the polypeptide is a recombinant polypeptide.

9. The composition of claim 8, wherein said recombinant polypeptide is a fusion polypeptide.

10. The composition of claim 1, wherein said nucleic acid molecule is selected from hybridization probes, primers and anti-sense oligonucleotides.

11. The composition of claim 1 which is a diagnostic composition.

12. The composition of claim 1 which is a therapeutic composition.

13. The composition of claim 1 for the manufacture of an agent for detecting and/or verifying, for the treatment, alleviation and/or prevention of an disorders, including metabolic diseases such as obesity and other body-weight regulation disorders as well as related disorders such as eating disorder, cachexia, diabetes mellitus, hypertension, coronary heart disease, hypercholesterolemia, dyslipidemia, osteoarthritis, gallstones, cancer, e.g. cancers of the reproductive organs, and sleep apnea and others, in cells, cell masses, organs and/or subjects.

14. Use of a nucleic acid molecule of the Men, GST2, Rab-RP1, Csp, F-box protein Lilina/FBL7, ABC50, coronin, Sec61 alpha, or VhaPPA1-1 gene family or a polypeptide encoded thereby or a fragment or a variant of said nucleic acid molecule or said polypeptide or an antibody, an aptamer or another receptor recognizing a nucleic acid molecule of the Men, GST2, Rab-RP1, Csp, F-box protein Lilina/FBL7, ABC50, coronin, Sec61 alpha, or VhaPPA1-1 gene family or a polypeptide encoded thereby for controlling the function of a gene and/or a gene product which is influenced and/or modified by a Men, GST2, Rab-RP1, Csp, F-box protein Lilina/FBL7, ABC50, coronin, Sec61 alpha, or VhaPPA1-1 homologous polypeptide.

15. Use of the nucleic acid molecule of the Men, GST2, Rab-RP1, Csp, F-box protein Lilina/FBL7, ABC50, coronin, Sec61 alpha, or VhaPPA1-1 gene family or a polypeptide encoded thereby or a fragment or a variant of said nucleic acid molecule or said polypeptide or an antibody, an aptamer or another receptor recognizing a nucleic acid molecule of the Men, GST2, Rab-RP1, Csp, F-box protein Lilina/FBL7, ABC50, coronin, Sec61 alpha, or VhaPPA1-1 protein gene family or a polypeptide encoded thereby for identifying substances capable of interacting with a Men, GST2, Rab-RP1, Csp, F-box protein Lilina/FBL7, ABC50, coronin, Sec61 alpha, or VhaPPA1-1 homologous polypeptide.

16. A non-human transgenic animal exhibiting a modified expression of a Men, GST2, Rab-RP1, Csp, F-box protein Lilina/FBL7, ABC50, coronin, Sec61 alpha, or VhaPPA1-1 homologous polypeptide.

17. The animal of claim 16, wherein the expression of the Men, GST2, Rab-RP1, Csp, F-box protein Lilina/FBL7, ABC50, coronin, Sec61 alpha, or VhaPPA1-1 homologous polypeptide is increased and/or reduced.

18. A recombinant host cell exhibiting a modified expression of a Men, GST2, Rab-RP1, Csp, F-box protein Lilina/FBL7, ABC50, coronin, Sec61 alpha, or VhaPPA1-1 homologous polypeptide.

19. The cell of claim 18 which is a human cell.

20. A method of identifying a (poly)peptide involved in the regulation of energy homeostasis and/or metabolism of triglycerides in a mammal comprising the steps of
(a) contacting a collection of (poly)peptides with a Men, GST2, Rab-RP1, Csp, F-box protein Lilina/FBL7, ABC50, coronin, Sec61 alpha, or VhaPPA1-1 homologous polypeptide or a fragment thereof under conditions that allow binding of said (poly)peptides;
(b) removing (poly)peptides which do not bind and
(c) identifying (poly)peptides that bind to said Men, GST2, Rab-RP1, Csp, F-box protein Lilina/FBL7, ABC50, coronin, Sec61 alpha, or VhaPPA1-1 homologous polypeptide.

21. A method of screening for an agent which modulates the interaction of a Men, GST2, Rab-RP1, Csp, F-box protein Lilina/FBL7, ABC50, coronin, Sec61 alpha, or VhaPPA1-1 homologous polypeptide with a binding target/agent, comprising the steps of
(a) incubating a mixture comprising
(aa) a Men, GST2, Rab-RP1, Csp, F-box protein Lilina/FBL7, ABC50, coronin, Sec61 alpha, or VhaPPA1-1 homologous polypeptide, or a fragment thereof;
(ab) a binding target/agent of said Men, GST2, Rab-RP1, Csp, F-box protein Lilina/FBL7, ABC50, coronin, Sec61 alpha, or VhaPPA1-1 homologous polypeptide or fragment thereof; and
(ac) a candidate agent under conditions whereby said Men, GST2, Rab-RP1, Csp, F-box protein Lilina/FBL7, ABC50, coronin, Sec61 alpha, or VhaPPA1-1 polypeptide or fragment thereof specifically binds to said binding target/agent at a reference affinity;
(b) detecting the binding affinity of said Men, GST2, Rab-RP1, Csp, F-box protein Lilina/FBL7, ABC50, coronin, Sec61 alpha, or VhaPPA1-1 polypeptide or fragment thereof to said binding target to determine a (candidate) agent-biased affinity; and
(c) determining a difference between (candidate) agent-biased affinity and reference affinity.

22. A method of screening for an agent which modulates the activity of a Men, GST2, Rab-RP1, Csp, F-box protein Lilina/FBL7, ABC50, coronin, Sec61 alpha, or VhaPPA1-1 homologous polypeptide comprising the steps of
(a) incubating a mixture comprising
(aa) a Men, GST2, Rab-RP1, Csp, F-box protein Lilina/FBL7, ABC50, coronin, Sec61 alpha, or VhaPPA1-1 homologous polypeptide, or a fragment thereof and
(ab) a candidate agent under conditions whereby said Men, GST2, Rab-RP1, Csp, F-box protein Lilina/FBL7, ABC50, coronin, Sec61 alpha, or VhaPPA1-1 polypeptide or fragment thereof exhibits a reference activity;
(b) detecting the activity of said Men, GST2, Rab-RP1, Csp, F-box protein Lilina/FBL7, ABC50, coronin, Sec61 alpha, or VhaPPA1-1 polypeptide or fragment thereof to determine a (candidate) agent-biased activity; and
(c) determining a difference between (candidate) agent-biased activity and reference activity.

23. (Cancelled)

24. (Cancelled)

25. Use of a (poly)peptide as identified by the method of claim 20 for the preparation of a pharmaceutical composition for the treatment, alleviation and/or prevention of diseases and disorders, including metabolic diseases such as obesity and other body-weight regulation disorders as well as related disorders such as eating disorder, cachexia, diabetes mellitus, hypertension, coronary heart disease, hypercholesterolemia, dyslipidemia, osteoarthritis, gallstones, cancer, e.g. cancers of the reproductive organs, and sleep apnea and other diseases and disorders.

26. Use of a nucleic acid molecule of the Men, GST2, Rab-RP1, Csp, F-box protein Lilina/FBL7, ABC50, coronin, Sec61 alpha, or VhaPPA1-1 family or of a fragment thereof for the preparation of a non-human animal which over- or under-expresses the Men, GST2, Rab-RP1, Csp, F-box protein Lilina/FBL7, ABC50, coronin, Sec61 alpha, or VhaPPA1-1 gene product.

27. Kit comprising at least one of
(a) a Men, GST2, Rab-RP1, Csp, F-box protein Lilina/FBL7, ABC50, coronin, Sec61 alpha, or VhaPPA1-1 nucleic acid molecule or a fragment thereof;
(b) a vector comprising the nucleic acid of (a);
(c) a host cell comprising the nucleic acid of (a) or the vector of (b);
(d) a polypeptide encoded by the nucleic acid of (a);
(e) a fusion polypeptide encoded by the nucleic acid of (a);
(f) an antibody, an aptamer or another receptor against the nucleic acid of (a) or the polypeptide of (d) or (e) and
(g) an anti-sense oligonucleotide of the nucleic acid of (a).

28. Use of an agent as identified by the method of claim 21 for the preparation of a pharmaceutical composition for the treatment, alleviation and/or prevention of diseases and disorders, including metabolic diseases such as obesity and other body-weight regulation disorders as well as related disorders such as eating disorder, cachexia, diabetes mellitus, hypertension, coronary heart disease, hypercholesterolemia, dyslipidemia, osteoarthritis, gallstones, cancer, e.g. cancers of the reproductive organs, and sleep apnea and other diseases and disorders.
